(19) United States
(12) Patent Application Publication
(10) Pub. No.: US 2024/0002925 A1
(43) Pub. Date: Jan. 4, 2024

(54) METHODS, SYSTEMS AND KITS FOR POLYPEPTIDE PROCESSING AND ANALYSIS

(71) Applicant: BOARD OF REGENTS, THE UNIVERSITY OF TEXAS SYSTEM, Austin, TX (US)

(72) Inventors: Eric V. ANSLYN, Austin, TX (US); Edward MARCOTTE, Austin, TX (US); Cecil J. HOWARD, II, Austin, TX (US); Jagannath SWAMINATHAN, Austin, TX (US); Angela M. BARDO, Austin, TX (US); Brendan FLOYD, Austin, TX (US); Brandon HOSFORD, Austin, TX (US); Le ZHANG, Austin, TX (US); Emily Faith BABCOCK, Austin, TX (US); Caroline M. HINSON, Austin, TX (US)

(73) Assignee: BOARD OF REGENTS, THE UNIVERSITY OF TEXAS SYSTEM, Austin, TX (US)

(21) Appl. No.: 18/056,970

(22) Filed: Nov. 18, 2022

Related U.S. Application Data
(63) Continuation of application No. PCT/US2021/033077, filed on May 19, 2021.
(60) Provisional application No. 63/027,219, filed on May 19, 2020.

(51) Int. Cl. C12Q 1/6869

(52) U.S. Cl. CPC C12Q 1/6869

(57) ABSTRACT

Provided herein are compositions, systems, methods, and kits for peptide analysis, including peptide sequencing. Aspects of the present disclosure provide bifunctional reagents which may selectively couple to amino acids and selectively couple to detectable species. Aspects of the present disclosure further provide methods for using these bifunctional reagents to sequence and analyze peptides.

This application is a continuation of PCT Application No. PCT/US2021/033077, filed May 19, 2021, which claims the benefit of U.S. Provisional Patent Application No. 63/027,219 filed May 19, 2020, each of which are entirely incorporated herein by reference in their entirety.

This invention was made with government support under Grant no. R35 GM122480 awarded by the National Institutes of Health. The government has certain rights in the invention.

This application contains a Sequence Listing XML, which has been submitted electronically and is hereby incorporated by reference in its entirety. Said XML Sequence Listing, created on Sep. 20, 2023, is named UTSBP1234US_updated.xml and is 6,374 bytes in size.

### BACKGROUND

As genetic variations often do not manifest in detectable or straightforward phenotypic changes, proteomics can be essential for understanding organismal and systems level biology and biochemistry. Proteomics has been applied to a variety of areas of clinical and biochemical interest, including pathogenesis, development, prevention, and treatment of a wide range of diseases. Protein identification and broader-scale proteomic profiling are critical for drug development, protein discovery, biological interrogation, and taxonomic classification. However, many traditional forms of proteomic analysis suffer from low sensitivity or low throughput. For example, histology often provides an accurate and sensitive handle for identifying specific proteins but can be limited in its ability to multiplex or facilitate high-throughput analysis.

Conversely, mass spectrometric protein analysis often enables high-throughput proteomic profiling at the expense of limited sensitivity and narrow dynamic ranges for detection.

### SUMMARY

As recognized herein, an increase in the use of proteomic strategies to understand the biology of living systems generates an ongoing need for more effective, efficient, and accurate methods for protein identification. Significant progress has been made in nucleic acid sequencing, with many next-generation technologies enabling single-molecule sensitivity and high-throughput genome-level measurements, including rapid and parallelized genome and transcriptome sequencing. In contrast, proteomic analysis has lagged, bottlenecking many forms of biological characterization. For example, mass spectrometry often requires prior knowledge of sample composition, can be incapable of detecting cell-to-cell variations from complex samples, and is often blind to low abundance and low-copy number proteins. Furthermore, mass spectrometric detection often provides a limited dynamic range for detection, often identifying proteins over at most four orders of magnitude, and thus missing the majority of complexity from even the simplest of proteomes, which typically span greater than ten orders of magnitude in terms of concentration.

An unbiased protein sequencing method with a dynamic range that covers the full range of protein concentrations in a proteome may allow for improved identification and characterization of gene products and subcellular complexes. Optical sequencing systems, methods, and kits that provide techniques for assembling labels, reporters, or a combination thereof onto amino acid(s) of a peptide may improve the efficiency of rapid single molecule sequencing of peptides.

Methods, systems, and kits of the present disclosure may advance current peptide or protein sequencing methods using optical techniques. Methods and systems of the present disclosure may overcome or alleviate at least some of the disadvantages of other peptide sequencing methods by increasing sequencing efficiency and accuracy. This may be used, for example, in cancer diagnostics, neurological diagnostics, and endocrinological diagnostics.

An aspect of the present disclosure provides a system comprising a peptide, wherein the peptide is immobilized to at least one support; and wherein the peptide comprises an amino acid coupled to a label, wherein the label comprises (i) a first reactive group that is configured to couple to a second reactive group that is coupled to a reporter moiety configured to emit a signal or (ii) a protecting group configured to prevent coupling between the label and the second reactive group.

Further aspects of the present disclosure provides a system for processing or analyzing a peptide, comprising: a peptide comprising an amino acid coupled to a first reactive group; and a support coupled to a second reactive group, wherein the first reactive group is configured to couple to the second reactive group to immobilize the peptide adjacent to the support.

In some embodiments, the at least one support is a bead, a polymer matrix, or an array. In some embodiments, the array is a microscopic slide. In some embodiments, an N-terminus of the peptide is coupled to the at least one support. In some embodiments, the at least one support is a bead.

In some embodiments, the N-terminus of the peptide is coupled to a cleavable unit, wherein the cleavable unit is coupled to the at least one support. In some embodiments, the cleavable unit comprises at least one of (i) a cleavable moiety, (ii) an aldehyde, (iii) the at least one support, or (iv) a spacer. In some embodiments, wherein the cleavable unit comprises (i)-(iv).

In some embodiments, the C-terminus of the peptide is coupled to the at least one support. In some embodiments, the at least one support is a microscopic slide. In some embodiments, the C-terminus is modified with a compound comprising an alkyne or an azide. In some embodiments, the alkyne or the azide are configured to couple to the at least one support.

In some embodiments, the C-terminus is an acidic amino acid, wherein the C-terminus comprises a first acidic residue and a second acidic residue. In some embodiments, the first acidic residue is a C-terminal carboxylic acid. In some embodiments, the second acidic residue is an aspartic acid side chain or a glutamic acid side chain. In some embodiments, the first acidic residue and the second acidic residue are modified with a compound comprising an alkyne or an azide. In some embodiments, the alkyne or the azide are configured to couple to the at least one support.

In some embodiments, an N-terminus of the peptide is coupled to a first support of the at least one support, wherein the first support is a bead, and wherein a C-terminus of the peptide is coupled to a second support of the at least one support, wherein the second support is a microscopic slide.

In some embodiments, the reporter moiety is configured to emit the signal upon excitation. In some embodiments, the signal is a detectable signal. In some embodiments, the detectable signal is an optical signal. In some embodiments, the reporter moiety comprises a fluorescent dye. In some embodiments, the reporter moiety comprises a spacer. In some embodiments, the protecting group does not emit an optically detectable signal.

In some embodiments, the amino acid is selected from the group consisting of lysine, cysteine, glutamic acid, aspartic acid, tyrosine, arginine, histidine, threonine, serine, proline, asparagine, glutamine, and tryptophan. In some embodiments, the amino acid comprises a post-translational modification. In some embodiments, the post-translational modification is selected from the group consisting of glycosylation, acetylation, alkylation, biotinylation, glutamylation, glycosylation, isoprenylation, phosphorylation, lipolation, phosphopantetheinylation, sulfation, selenation, amidation, ubiquitination, hydroxylation, nitration, nitrosylation, citrullination, N-terminal glutamine cyclization, N-terminal glutamate cyclization, and SUMOylation.

In some embodiments, the peptide comprises a plurality of amino acids coupled to a plurality of labels. In some embodiments, the plurality of amino acids comprise a first amino acid coupled to a first label and a second amino acid coupled to a second label. In some embodiments, the plurality of amino acids comprises (i) a plurality of first amino acids coupled to a plurality of first labels and (ii) a plurality of second amino acids coupled to a plurality of second labels. In some embodiments, the first label couples only to the first amino acid and the second label couples only to the second amino acid. In some embodiments, at least one label of the plurality of labels is coupled to a specific amino acid type of the plurality of amino acids. In some embodiments, at least one label of the plurality of labels is configured to react with a specific second reactive group coupled to a specific reporter moiety.

In some embodiments, the peptide comprises a plurality of amino acids. In some embodiments, at least one amino acid of the plurality of amino acids is selected from the group consisting of lysine, cysteine, glutamic acid, aspartic acid, tyrosine, arginine, histidine, threonine, serine, glutamine, asparagine and tryptophan. In some embodiments, the at least one amino acid is coupled to the label.

In some embodiments, the first reactive group is selected from the group consisting of an azide, an alkyne, an alkene, an aldehyde, a ketone, a tetrazine, a thiol, a dithiol, a cyclooctene, and norbornene. In some embodiments, the alkyne is a strained alkyne. In some embodiments, the first reactive group is selected from the group consisting of the azide, the alkene, the aldehyde, the ketone, and the tetrazine. In some embodiments, the second reactive group is selected from the group consisting of an alkyne, an azide, a thiol, a dithiol, a cyclooctene, an alkene, an aldehyde, a ketone, a tetrazine, and norbornene. In some embodiments, the first reactive group is selected from the group consisting of the alkyne, the thiol, the dithiol, and the cyclooctene. In some embodiments, the alkyne is a strained alkyne.

Another aspect of the present disclosure provides a method for processing or analyzing a peptide, comprising: (a) providing the peptide comprising an amino acid coupled to a label, wherein the label comprises (i) a first reactive group that is configured to couple to a second reactive group that is coupled to a reporter moiety configured to emit a signal or (ii) a protecting group configured to prevent coupling between the label and the second reactive group; (b) bringing the peptide in contact with a mixture comprising the second reactive group; (c) with the peptide immobilized to at least one support, detecting a signal from the peptide; and (d) using the signal or signal change to identify the amino acid or an additional amino acid of the peptide.

Another aspect of the present disclosure provides a method for labeling an amino acid of a peptide, comprising providing the peptide, wherein the peptide comprises an internal amino acid coupled to an azide and a C-terminus coupled to an alkyne; and bringing the peptide in contact with a first reporter under conditions such that the first reporter reacts with the internal amino acid, wherein the first reporter comprises a strained alkyne. In some embodiments, the method of is performed in the absence of copper (Cu). In some embodiments, the method further comprises reacting a second reporter different from the first reporter with the C-terminus, wherein the second reporter comprises a non-strained alkyne. In some embodiments, the method is performed in the presence of copper (Cu). In some embodiments, the azide coupled to the internal amino acid does not react with the alkyne coupled to the C-terminus.

In some embodiments, the method comprises immobilizing the peptide prior to (c). In some embodiments, the peptide is immobilized to the at least one support in (a) or (b). In some embodiments, the method comprises immobilizing the peptide subsequent to (b). In some embodiments, in (a), the label comprises the first reactive group, and in (b), the second reactive group reacts with the first reactive group.

In some embodiments, the peptide is provided as a plurality of peptides. In some embodiments, the peptide comprises at least one peptide of the plurality of peptides. In some embodiments, the plurality of peptides is immobilized to a plurality of supports. In some embodiments, the plurality of peptides comprises a first peptide and a second peptide.

In some embodiments, the first peptide comprises a first amino acid coupled to a first label and the second peptide comprises a second amino acid coupled to a second label. In some embodiments, the first label is configured to react with the second reactive group and the second label is configured to react with a different second reactive group than the second reactive group. In some embodiments, the different second reactive group is coupled to a second reporter moiety configured to emit a different signal than the reporter moiety.

In some embodiments, the peptide comprises a plurality of amino acids coupled to a plurality of labels. In some embodiments, the plurality of amino acids comprises a first amino acid coupled to a first label and a second amino acid coupled to a second label. In some embodiments, the plurality of amino acids comprises a plurality of first amino acids coupled to a plurality of first labels and a plurality of second amino acids coupled to a plurality of second labels. In some embodiments, the first label couples only to the first amino acid and the second label couples only to the second amino acid. In some embodiments, at least one label of the plurality of labels is coupled to a specific amino acid type of the plurality of amino acids. In some embodiments, at least one label of the plurality of labels is configured to react with a specific second reactive group coupled to a specific reporter moiety.

In some embodiments, the peptide comprises a plurality of amino acids. In some embodiments, at least one amino acid of the plurality of amino acids is selected from the group consisting of lysine, cysteine, glutamic acid, aspartic acid, tyrosine, arginine, histidine, threonine, serine, asparagine, glutamine, and tryptophan. In some embodiments, the at least one amino acid is coupled to the label. In some embodiments, the plurality of amino acids comprises at least two amino acid types. In some embodiments, less than all of the amino acid types of the plurality of amino acids are labelled.

In some embodiments, the at least two amino acid types comprise a first amino acid type and a second amino acid type. In some embodiments, the first amino acid type is coupled to a first label and wherein the second amino acid type is coupled to a second label. In some embodiments, the first label and the second label are each coupled to a different reporter moiety. In some embodiments, each amino acid type of the at least two amino acid types are coupled to a different label. In some embodiments, the peptide comprises at least four amino acid types, wherein each amino acid type of the at least four amino acid types are coupled to a different label. In some embodiments, less than all of the plurality of amino acids are labelled.

In some embodiments, the method comprises (e), subjecting the peptide to conditions sufficient to remove at least one amino acid from the peptide immobilized to the at least one support. In some embodiments, the conditions sufficient to remove at least one amino acid comprise an Edman degradation agent or an organophosphate-containing agent. In some embodiments, subsequent to (e), the amino acid coupled to the label becomes a terminal amino acid. In some embodiments, the at least one amino acid is removed from an N-terminus of the peptide.

In some embodiments, the signal or signal change is used to identify at least a portion of a sequence of the peptide. In some embodiments, the method comprises (f), repeating (d) and (e) to detect at least one additional signal or signal change from the peptide immobilized to the at least one support and (ii) using the signal or signal change and the additional signal or signal change to identify at least a portion of a sequence of the peptide immobilized to the at least one support.

In some embodiments, the at least one signal or signal change is detected with an optical detector having single-molecule sensitivity. In some embodiments, the at least one signal or signal change comprises a plurality of signals of different frequencies or frequency ranges. In some embodiments, the at least one signal or signal change comprises a plurality of signals of different intensities. In some embodiments, the at least one signal or signal change is an optical signal.

In some embodiments, the reporter moiety generates the at least one signal or signal change. In some embodiments, the reporter moiety comprises a dye, which dye generates the at least one signal or signal change. In some embodiments, the protecting group comprises one or more groups, each group being independently selected from the group consisting of azide, alkyl, alkylene, aryl, heteroaryl, heteroaryl-alkyl, and aryl-alkyl.

Another aspect of the present disclosure provides method for processing or analyzing a peptide, comprising: (a) providing the peptide comprising an amino acid coupled to a first reactive group that is configured to couple to a second reactive group coupled to a support; (b) bringing the peptide in contact with the second reactive group to permit the first reactive group to couple to the second reactive group, thereby immobilizing the peptide to the support; (c) with the peptide immobilized to the support, detecting a signal from a label coupled to an amino acid of the peptide; and (d) using the signal or change thereof to identify the amino acid.

In some embodiments, the first reactive group is coupled to the amino acid via a functional group. In some embodiments, the functional group is coupled to a reactive side chain of the amino acid. In some embodiments, (a) further comprises coupling the amino acid to a functional group coupled to the first reactive group. In some embodiments, the functional group only couples to the amino acid coupled to a first reactive group that is configured to couple to a second reactive group coupled to a support.

In some embodiments, the amino acid is selected from the group consisting of lysine, cysteine, glutamic acid, aspartic acid, tyrosine, arginine, histidine, threonine, serine, proline, glutamine, and tryptophan. In some embodiments, the first reactive group is selected from the group consisting of an azide, an alkyne, an alkene, an aldehyde, a ketone, a tetrazine, a thiol, a dithiol, an ester, a cyclooctene, and norbornene or activated ester. In some embodiments, the ester is an activated ester. In some embodiments, the activated ester is EDC-carbodiimide.

In some embodiments, the first reactive group is the azide or the alkyne. In some embodiments, the second reactive group is selected from the group consisting of an alkyne, an azide, a thiol, a dithiol, a cyclooctene, an alkene, an aldehyde, a ketone, a tetrazine, and norbornene. In some embodiments, the second reactive group is the alkyne or the azide. In some embodiments, the first reactive group and the second reactive group are coupled via a Click reaction. In some embodiments, the second reactive group is coupled to the support through a linker. In some embodiments, the linker is a bond or an optionally substituted alkylene or an optionally substituted heteroalkylene. In some embodiments, the support is an array. In some embodiments, the support is a bead. In some embodiments, the amino acid coupled to a first reactive group is a terminal amino acid.

In some embodiments, the amino acid coupled to a first reactive group is an internal amino acid. In some embodiments, the amino acid in (a) is the same as the amino acid coupled to the label in (c). In some embodiments, the amino acid in (a) is different from the amino acid coupled to the label in (c). In some embodiments, (c) further comprises coupling the label to the amino acid of the peptide prior to (b). In some embodiments, (c) further comprises coupling the label to the amino acid of the peptide subsequent to (b).

In some embodiments, the label is coupled to reporter moiety. In some embodiments, the reporter moiety is coupled to a third reactive group or an antibody. In some embodiments, the third reactive group or the anti-body is configured to couple to the amino acid of the peptide in (c). In some embodiments, the amino acid of the peptide in (c) is a terminal amino acid. In some embodiments, the amino acid of the peptide in (c) is an internal amino acid. In some embodiments, the reporter moiety is coupled to a linker, wherein the antibody couples to the amino acid of the peptide in (c).

Another aspect of the present disclosure provides a kit for assaying a sequence of a peptide in a sample, comprising: a reporter; a label comprising a first reactive group and (i) a second reactive group or (ii) a protecting group, wherein the first reactive group is configured to couple to an amino acid of an amino acid type, wherein the second reactive group is configured to couple to the reporter, comprising a reporter moiety configured to emit a signal, and wherein the protecting group is configured to prevent coupling between the label and the reporter moiety; and instructions for using the label to process the peptide to provide the peptide comprising the amino acid coupled to the label.

In some embodiments, the reporter is coupled to a third reactive group configured to react with the second reactive group. In some embodiments, the reporter is configured to emit the signal upon excitation. In some embodiments, the reporter comprises a fluorescent dye. In some embodiments, the reporter comprises a spacer. In some embodiments, the protecting group does not emit an optically detectable signal.

In some embodiments, the kit comprises a protein capture agent. In some embodiments, the protein capture agent comprises a solid support coupled to a cleavable linker, wherein the cleavable linker is coupled to an aldehyde. In some embodiments, the protein capture agent is configured to couple to a N-terminus of the peptide. In some embodiments, the solid support is a bead. In some embodiments, the aldehyde is pyridinecarbaldehyde (PCA) or a derivative thereof.

In some embodiments, the kit further comprises a surface attachment agent. In some embodiments, the surface attachment agent comprises an alkyne or an azide. In some embodiments, the surface attachment agent is configured to couple to a C-terminus of the peptide. In some embodiments, the kit further comprises a support to which the surface attachment agent attaches. In some embodiments, the support is a microscopic slide. In some embodiments, the kit further comprises at least one protease, at least one digestion reagent, and solid support beads.

In some embodiments, the method further comprises, subsequent to (c), subjecting the peptide to conditions sufficient to remove an amino acid from a terminal end of the peptide. In some embodiments, the conditions are Edman degradation conditions. In some embodiments, the method further comprises repeating (c) and (d) one or more times to identify a signal pattern of a plurality of amino acids of the peptide, which the signal pattern of the plurality of amino acids comprises the signal or change thereof in (d). In some embodiments, the method further comprises using the signal pattern of the plurality of amino acids to obtain a sequence of the peptide.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A shows an example of a peptide configured to couple to a first support, a second support, and a label configured to couple to a reporter moiety or a protecting group.

FIG. 1B illustrates a peptide coupled to a first support through its N-terminus, a second support through its C-terminus, and multiple labels through internal amino acids.

FIG. 2 shows an example of a method for analyzing a peptide comprising capturing the peptide on a support via the N-terminal amino acid, coupling at least one label to the peptide, releasing the labeled peptides from the support, and coupling the labeled peptides to a surface.

FIG. 3 provides an example of a kit comprising a plurality of amino acid specific label disposed in an array.

FIG. 4 shows an example of a kit comprising amino acid specific labels and a variety of reporter moieties and protecting groups, and an array disposed with separate combinations of labels and amino acid. Each combination of label and label may be further combined with a number of reporter moieties.

FIG. 5 shows an example for a method for sequencing peptides using N-terminal amino acid binding agents (NAABs).

FIG. 6 shows a computer system that is programmed or otherwise configured to implement methods provided herein.

FIG. 7 illustrates method for labeling and immobilizing a peptide.

FIG. 8 illustrates a workflow for capturing peptides on a lantern.

FIG. 9 outlines a kit for labeling peptides with bifunctional, amino acid-specific labels.

FIG. 10 provides a kit for selectively labeling peptides with amino acid-specific labels.

FIG. 11 provides an example workflow for computationally designing and then using a label-fluorophore kit of the present disclosure.

FIG. 12A-D provide fluorescence decrease results for a fluorosequencing experiment with Edman degradation (E1-E7) and non-degradation (M1-M4) steps. The type of amino acid labeled in each experiment is shown to the left of each figure. FIG. 12A was performed with labeled cysteine. FIG. 12B was performed with labeled glutamate. FIG. 12C was performed with labeled phosphoserine. FIG. 12D was performed with labeled lysine.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “analyte,” as used herein, generally refers to a substance (e.g., a molecule) whose presence or absence is measured or identified. An analyte can be a substance (e.g., molecule) for which a detectable probe may be used to identify the presence or absence of such substance. As a non-limiting example, an analyte can be a macromolecule, such as, for example, a peptide or a protein. An analyte can be part of a sample that contains or is suspected of containing other components, or can be the sole or the major component of the sample. An analyte can be a component of a whole cell or tissue, a cell or tissue extract, a fractionated lysate or a cell or tissue, or a substantially purified molecule. In some embodiments, the analyte is a peptide.

The terms “polypeptide” and “peptide,” as used interchangeably herein, generally to refer to a polymer of amino acids in which an amino acid may be linked to another amino acid by a peptide bond. In some examples, a peptide is a protein. The amino acid may be a naturally occurring amino acid or a non-naturally occurring amino acid (e.g., an amino acid analogue). The peptide can be linear or branched. The peptide can include modified amino acids. The peptide may be interrupted by non-amino acids. A peptide can occur as a single chain or an associated chain. The peptide may include a plurality of amino acids. The peptide may have a secondary and tertiary structure (e.g., the peptide may be a protein comprising defined secondary, tertiary, and quaternary structures). In some examples, the peptide comprises at least 2, 3, 4, 5, 6, 7, 8, 9, 10, 20, 30, 40, 50, 100, 1000, 10,000, or more amino acids. The peptide may be a fragment of a larger polymer. In some examples, the peptide is a fragment of a larger peptide, such as a fragment of a protein.

The term “amino acid,” as used herein, generally refers to a naturally occurring or non-naturally occurring amino acid (e.g., an amino acid analogue). The non-naturally occurring amino acid may be an engineered or synthesized amino acid. An amino acid may contain a “side chain”, which may differentiate amino acid types from one another.

The terms “amino acid sequence,” “peptide sequence,” and “polypeptide sequence,” as used herein, generally refer to a sequence of at least two amino acids or amino acid analogs that are covalently linked (e.g., by a peptide (amide) bond or an analog of a peptide bond). A peptide sequence may refer to a complete sequence or a portion of a sequence. For example, a peptide sequence may contain gaps, positions with unknown identities, or positions that can accommodate distinct species.

As used herein, the term “side chain” generally refers to a structure attached to an alpha carbon (attaching an amine and a carboxylic acid group of an amino acid) that may be unique to each type of amino acid. A side chain may have a certain shape, size, charge, reactivity, or a combination thereof. A side chain may contain a basic moiety (e.g., the guanidino group in arginine), an acidic moiety (e.g., the carboxylic acid in aspartic acid), a polar moiety (e.g., the hydroxyl groups in serine, threonine, and tyrosine), a hydrophobic moiety (e.g., the alkyl groups in leucine, isoleucine, alanine, and valine), or any combination thereof. In some cases, an amino acid contains more than one side chain. The side chain may be or include hydrogen, an alkyl group, a hydroxyl group, an aryl group, a heteroaryl group, a carboxylic acid, an amide, an amine, a guanidine, a thiol, a thioether, a selenol, or any combination thereof. In some instances, the side chain is a hydrogen (an amino acid with a hydrogen side chain may be, e.g., glycine).

The term “cleavable unit,” as used herein, generally refers to a moiety of a molecule that can be used to split or dissociate the molecule into two or more other molecules. A cleavable unit may be split under cleavage conditions. Non-limiting examples of cleavage conditions include use of: enzymes, nucleophilic or basic reagents, reducing agents, photo-irradiation, electrophilic or acidic reagents, organometallic or metal reagents, and oxidizing reagents.

The term “sample,” as used herein, generally refers to a chemical or biological sample containing or suspected of containing a peptide. For example, a sample can be a biological sample containing one or more peptides. The biological sample can be obtained (e.g., extracted or isolated) from or include blood (e.g., whole blood), plasma, serum, urine, saliva, mucosal excretions, sputum, stool and tears. The biological sample can be a fluid or tissue sample (e.g., skin sample). In some examples, the sample is derived from a homogenized tissue sample (e.g., brain homogenate, liver homogenate, kidney homogenate). In some embodiments, the sample is taken from a specific type of cell (e.g., neuronal cell, muscle cell, liver cell, kidney cell). The sample may be acquired from a diseased cell or tissue (e.g., a tumor cell, a necrotic cell). In some examples, the sample is from a disease-associated inclusion (e.g., a plaque, a biofilm, a tumor, a non-cancerous growth). In some examples, the sample is obtained from a cell-free bodily fluid, such as whole blood, saliva, or urine. In some examples, the sample can include circulating tumor cells. In some examples, the sample is an environmental sample (e.g., soil, waste, ambient air), industrial sample (e.g., samples from any industrial processes), and food samples (e.g., dairy products, vegetable products, and meat products). The sample may be processed prior to loading into a microfluidic device. For example, the sample may be processed to purify the peptides and/or to include reagents.

As used herein, the term “support” generally refers to an entity to which a substance (e.g., molecular construct) can be immobilized. The solid may be a solid or semi-solid (e.g., gel) support. As a non-limiting example, a support may be a bead, a polymer matrix, an array, a microscopic slide, a glass surface, a plastic surface, a transparent surface, a metallic surface, a magnetic surface, a multi-well plate, a nanoparticle, a microparticle, a lantern, or a functionalized surface. The support may be planar. As an alternative, the support may be non-planar, such as including one or more wells. A bead can be, for example, a marble, a polymer bead (e.g., a polysaccharide bead, a cellulose bead, a synthetic polymer bead, a natural polymer bead), a silica bead, a functionalized bead, an activated bead, a barcoded bead, a labeled bead, a PCA bead, a magnetic bead, or a combination thereof. A bead may be functionalized with a functional motif. Some non-limiting examples of functional motifs include a capture reagent (e.g., pyridinecarboxyaldehyde (PCA)), a biotin, a streptavidin, a strep-tag II, a linker, or a functional group that can react with a molecule (e.g., an aldehyde, a phosphate, a silicate, an ester, an acid, an amide, an alkyne, an azide, or an aldehyde dithiolane. The functional group may couple specifically to an N-terminus or a C-terminus of a peptide. The functional group may couple specifically to an amino acid side chain. The functional group may couple to a side chain of an amino acid (e.g., the acid of a glutamate or aspartate, the thiol of a cysteine, the amine of a lysine, or the amide of a glutamine, or asparagine). The functional group may couple specifically to a reactive group on a particular species, such as a label. In some examples of functionalized beads, the functional motif can be reversibly coupled and cleaved. A functional motif can also irreversibly couple to a molecule.

As used herein, the term “Edman degradation” generally refers to a method of removing an amino acid from the N-terminal end of a peptide using an isothiocyanate (e.g., phenyl isothiocyanate). Edman degradation may be coupled with various peptide sequencing and analysis methods. Edman degradation may be performed sequentially.

As used herein, the term “array” generally refers to a population of sites. Such populations of sites can be differentiated from one another according to relative location. Different molecules that are at different sites of an array can be differentiated from each other according to the locations of the sites in the array. An individual site of an array can include one or more molecules of a particular type. For example, a site can include a single peptide having a particular sequence or a site can include several peptides having the same sequence. The sites of an array can be different features located on the same substrate. Such features may include, without limitation, wells in a substrate, beads (or other particles) in or on a substrate, projections from a substrate, ridges on a substrate or channels in a substrate. The sites of an array can be separate substrates each bearing at least one molecule. Different molecules attached to separate substrates can be identified according to the locations of the substrates on a surface to which the substrates are associated or according to the locations of the substrates in a liquid or gel. Such different molecules may have the same or different sequences. An array may include one or more wells, and a well of the one or more wells may have one or more beads. As an alternative, the array may be a planar surface having, for example, a molecule immobilized thereon, or, as another example, one or more beads immobilized thereon.

As used herein, the term “label” generally refers to a molecular or macromolecular construct that can couple to a reactive group. The label may comprise at least one reactive group (e.g., a first reactive group and a second reactive group). The at least one reactive group may be configured to couple to a peptide. The at least one reactive group may be configured to couple to a support. The at least one reactive group may be configured to couple to a reporter moiety. A label may provide a measurable signal.

As used herein, the term “polymer matrix” generally refers to a (e.g., continuous) phase material that comprises at least one polymer. In some embodiments, the polymer matrix refers to the at least one polymer as well as the interstitial space not occupied by the polymer. A polymer matrix may be composed of one or more types of polymers. A polymer matrix may include linear, branched, and crosslinked polymer units. A polymer matrix may also contain non-polymeric species intercalated within its interstitial spaces not occupied by polymer chains. The intercalated species may be solid, liquid or gaseous species. For example, the term ‘polymer matrix’ may encompass desiccated hydrogels, hydrated hydrogels, and hydrogels containing glass fibers.

The term “reporter moiety,” as used herein, generally refers to an agent that generates a measurable signal. Such a signal may include, but is not limited to, fluorescence (e.g., a dye), visible light, motion (e.g., a mass tag), radiation, or a nucleic acid sequence (e.g., a barcode). Such a signal may include, but is not limited to, fluorescence, phosphorescence, or, radiation. Such signal may be light (or electromagnetic radiation). The light may include a frequency or frequency distribution in the visible portion of the electromagnetic spectrum. For example, the light may be infrared or ultraviolet light. The signal may be an electrostatic, a conductive, or an impedance signal. The signal may be a charge. A “reporter” may comprise a “reporter moiety”. The reporter may comprise a reactive group. The reactive group may be configured to couple to a label.

Single molecule peptide sequencing may be used in various applications, such as, for example, protein engineering, organism engineering, and systems biology. Providing single molecule protein sequencing platforms with increased speed, accuracy, versatility and ease of use may accelerate research across a broad range of biological and chemical disciplines. Among the challenges associated with single molecule peptide sequencing are, for example, high user input requirements, inability to handle subject peptide complexity or modifications (such as post-translational modifications), and speed and ease of use.

Peptide sequence information may be obtained from a peptide molecule or from one or more portions of the peptide molecule. Peptide sequencing may provide complete or partial amino acid sequence information for a peptide sequence or a portion of a peptide sequence. At least a portion of the peptide sequence may be determined at the single molecule level. In some cases, partial amino acid sequence information, including for example, the relative positions of a specific type of amino acid (e.g., lysine) within a peptide or portion of a peptide, may be sufficient to uniquely identify an individual peptide molecule. For example, a pattern of amino acids, such as, for example, X-X-X-Lys-X-X-X-X-Lys-X-Lys (SEQ ID NO: 1), which indicates the distribution of lysine molecules within an individual peptide molecule, may be searched against a known proteome of a given organism to identify the individual peptide molecule. Such information may be used to identify a macromolecule (e.g., protein) from which the peptide was derived, and may preclude the need to identify all amino acids of the peptide.

Peptide sequencing may be used to acquire information (including, for example, amino acid sequence information) from individual peptide molecules in a mixture of diverse peptide molecules. A method of the present disclosure may comprise detecting a reporter moiety coupled to amino acids of a peptide or a plurality of peptides immobilized on a solid surface (including, for example, a glass slide, or a glass slide whose surface has been chemically modified, a plastic slide, a multi-well plate, a cassette). In some cases, the detecting comprises optical (e.g., fluorescence) detection. In some cases, the reporter moiety comprises a fluorescent group. In some cases, the reporter moiety comprises a plurality of amino acid-type specific labels coupled to a plurality of types of amino acids of the peptide or plurality of peptides. In some cases, the detecting comprises single-molecule (e.g., single peptide) sensitivity.

Numerous commercially available optical devices can be applied in this manner. For example, conventional microscopes equipped with total internal reflection illumination and intensified charge-couple device (CCD) detectors may be adapted for sequencing methods disclosed herein. A high sensitivity CCD camera may be configured to simultaneously record the fluorescence intensity of multiple individual (e.g., single) peptide molecules distributed across a surface, and may be coupled to an image splitter to facilitate the simultaneous collection of multiple, distinct images (e.g., a first image comprising light of a first wavelength and a second image comprising light of a second wavelength). Using a motorized microscope stage with automated focus control to image multiple stage positions in the flow cell may allow thousands, tens of thousands, hundreds of thousands, millions, or more individual single peptides to be analyzed (e.g., sequenced) in a single experiment.

The present disclosure provides a range of expedient and facile methods for analyzing a peptide. Additionally, some aspects of the present disclosure provide compositions that facilitate effective peptide characterization and analysis. Furthermore, in some aspects the present disclosure provides kits which enable effective peptide analysis.

### 1. Proteomics

Proteomics is the large-scale study proteins present in an organism, system, or biological consortia. Proteins are quintessential to organisms, facilitating the majority of chemical and physical processes carried out by life. Accordingly, the set of proteins expressed within a cell, organism, or system often strongly reflective of health, biological state, biological activity, and physical conditions (e.g., heat stress, nutrient depletion, or stimulation). Accordingly, peptide sequencing is a tool that may be used in a variety of applications within the field of proteomics.

The present disclosure provides methods and systems for peptide (e.g., protein) analysis (e.g., compositional analysis and sequencing). Methods of the present disclosure may permit a peptide (e.g., protein) to be analyzed (e.g., sequenced) in a manner that provides various non-limiting benefits, such as, for example, (i) sequencing a protein or peptide comprising a chemically modified N-terminal amino acid (e.g., ADP-ribosylation, fluorophores, etc.), (ii) sequencing a protein or peptide comprising an unnatural amino acid residue (e.g., 3-amino acid, peptoid, PNA, etc.), or (iii) minimizing cross-reactivity of dyes. Peptide sequencing may be used to reveal novel biomarkers for the diagnosis of cancer and other diseases or in understanding the function of healthy cells. Peptides produced by cells or tissues may act as unique biomarkers. Enhanced detection of these biomarkers through peptide sequencing may provide earlier, more accurate diagnoses of disease.

Provided herein are systems, methods, and kits that can be used to enhance detection of biomarkers by streamlining, optimizing, or otherwise improving the speed, efficiency, or accuracy with which a peptide can be processed or analyzed.

A method of the present disclosure may be configured to analyze peptides spanning at least 2, at least 3, at least 4, at least 5, at least 6, at least 7, at least 8, at least 9, or at least 10 orders of magnitude in concentration in a sample. For example, a method of the present disclosure may permit simultaneous measurements of immunoglobulins and cytokines from human serum, peptides that are traditionally difficult to simultaneously detect due to their 7+ order of magnitude concentration differences. A method of the present disclosure may be configured to identify at least 100, at least 500, at least 1000, at least 5000, at least 10⁴, at least 5×10⁴, at least 10⁵, or at least 5×10⁵ different proteins from a sample. A method of the present disclosure may be configured to identify at least 50, at least 100, at least 150, at least 200, at least 250, at least 300, at least 400, at least 500, at least 600, at least 700, at least 800, at least 900, at least 1000, at least 1200, at least 1500, at least 1800, at least 2000, at least 2500, at least 3000, at least 3500, at least 4000, or at least 5000 types of proteins from a sample (e.g., human lung homogenate). A method of the present disclosure may be configured to simultaneously (e.g., within a single assay) identify at least 50, at least 100, at least 150, at least 200, at least 250, at least 300, at least 400, at least 500, at least 600, at least 700, at least 800, at least 900, at least 1000, at least 1200, at least 1500, at least 1800, at least 2000, at least 2500, at least 3000, at least 3500, at least 4000, or at least 5000 types of proteins from a sample (e.g., buffy coat lysate). A method of the present disclosure may be configured to identify at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the types of peptides in a biological sample (e.g., a human biological sample). For example, a method of the present disclosure may comprise coupling cysteine-specific and lysine-specific labels to a plurality of peptides derived from a human urine sample, immobilizing about 10⁵ of the peptides to a glass slide, performing sequential rounds of label detection and N-terminal amino acid removal on the peptides, and comparing the identified cysteine and lysine peptide sequences against a database of known human urine peptides, thereby identifying at least 60% of the about 10⁵ peptides from the sample.

### 2. Amino Acid Selective Labels

The present disclosure provides a range of compositions, systems, and methods for selectively labeling amino acids, for example for peptide sequencing. Sample preparation may be improved by selectively labeling specific amino acid types (e.g., cysteine, lysine, histidine, tyrosine, threonine, serine, arginine, glutamate, aspartate, tryptophan, or any combination thereof) and amino acid positions (e.g., N-terminal or C-terminal amino acids). A label may comprise a first reactive group configured to couple to a specific amino acid type (e.g., arginine) or to a collection of amino acid types (e.g., lysine and cysteine).

A composition, system, or method of the present disclosure may selectively label cysteine, lysine, tyrosine, histidine, glutamic acid, aspartic acid, tyrosine, threonine, serine, arginine, N-terminal amines, C-terminal carboxyl-groups, or any combination thereof. A composition, system, or method may selectively label a group of amino acids, for example a specific maleimide reagent may be configured to couple to lysine and cysteine residues present in a sample. A composition, system, or method may selectively label a single amino acid, for example a specific epoxide reagent may be configured to selectively couple to histidine residues over other amino acid types.

The present disclosure provides a range of reagents for selectively labeling specific amino acid types (e.g., cysteine) and groups of amino acids (e.g., carboxylate side chain-containing amino acids, such as glutamate and aspartate). Non-limiting examples of cysteine-specific labels may include certain iodoacetamides, thiols, benzyl and allyl halides, selenocyanates, maleimides, and alkynes (e.g., certain alkynoic amides). In some cases, a maleimide may be configured to couple to cysteine and lysine. An example of a cysteine labeling scheme, in which a cysteine thiol nucleophilically couples to an iodoacetamide, is outlined in Scheme 2 below.

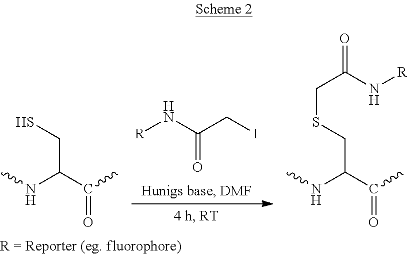

Non-limiting examples of lysine-specific labels may include certain thiocyanates and isothiocyanates, maleimides, aldehydes, isatoic anhydrides, and NHS esters. For example, a lysyl butylamine sidechain may be selectively coupled to an NHS ester, as outlined in Scheme 3.

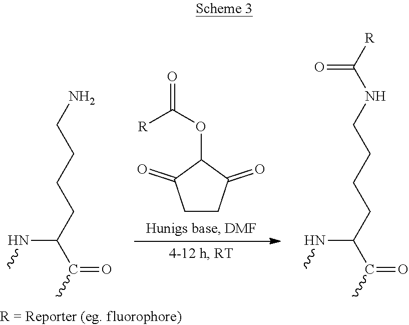

Peptide carboxylates (e.g., glutamate, aspartate, and C-terminal carboxylates) may be labeled through nucleophilic coupling steps. An example of such a coupling process is provided in Scheme 4, which illustrates carboxyl conversion to amide conversion via amine-based nucleophilic substitution.

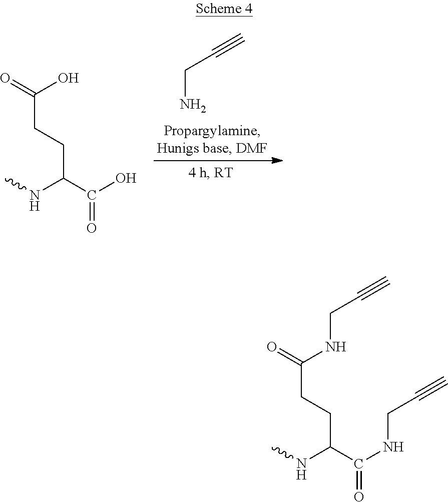

Non-limiting examples of tyrosine-specific labels may include certain aryl diazo compounds, Mannich reaction reagents (e.g., an amine and formaldehyde), and imines. Scheme 5 provides an example of tyrosine-specific labeling scheme comprising an aryl diazo compound.

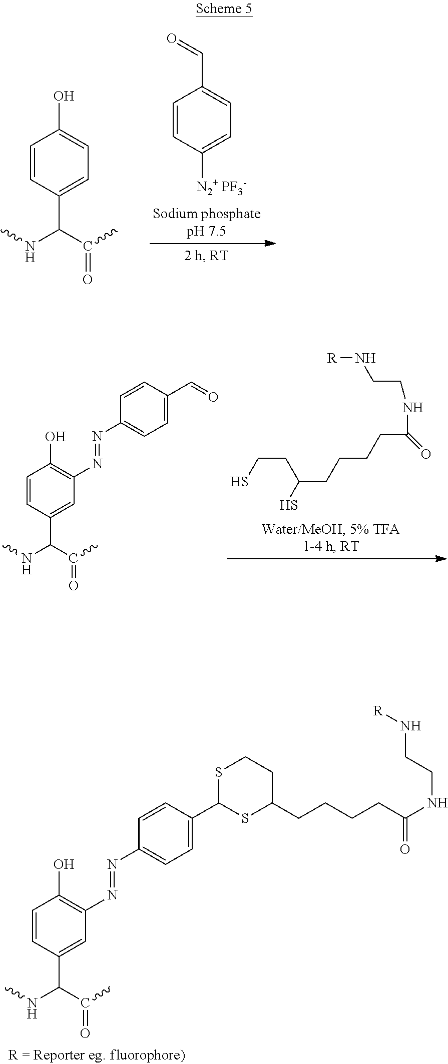

Non-limiting examples of histidine-specific labels may include certain α,β-unsaturated ketones and epoxides. An example of a histidine-specific labeling scheme is provided in Scheme 6, which illustrates histidine labeling with a 2-cyclohexenone reagent.

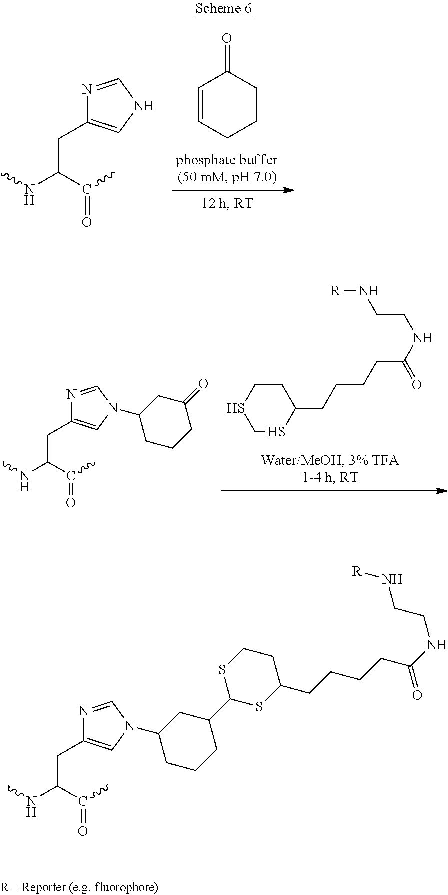

Non-limiting examples of arginine labeling include arginine guanidinium acylation and derivatization to pyrimidine with a glyoxal label. Scheme 7 illustrates an example of arginine labeling by an NHS ester with the aid of Barton's base.

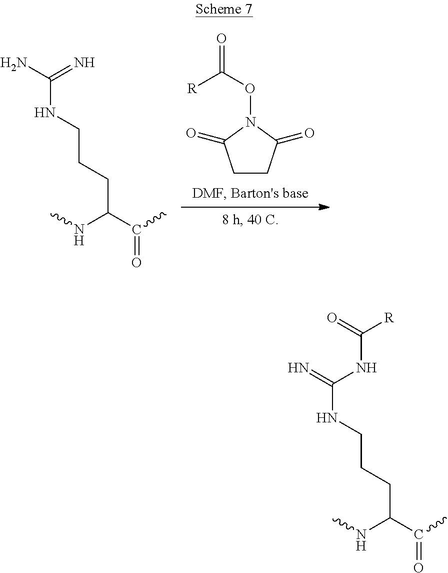

Non-limiting examples of arginine labels include benzophenones and oxaziridines, such as the oxaziridine labeling process outlined in Scheme 8.

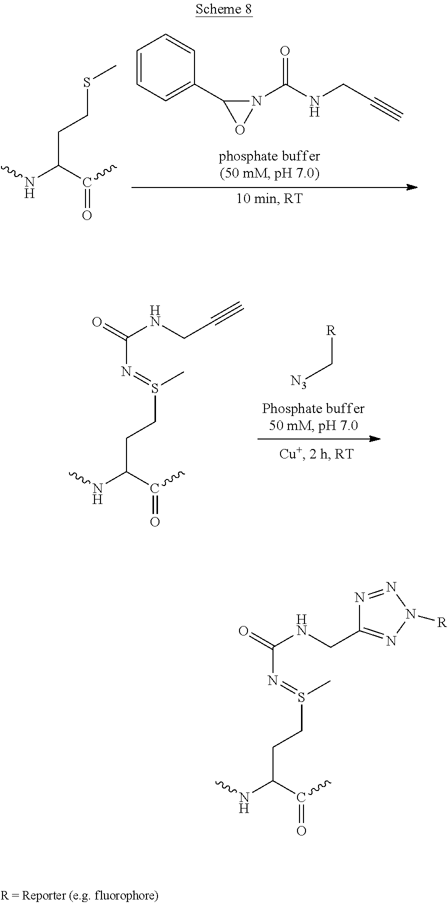

Non-limiting examples of tryptophan labels may include diazopropanoate esters and photoactivatable haloalkenes. Scheme 9 provides an example of tryptophan indole labeling with a diazopropanoate ester to yield an amine derivatized tryptophan.

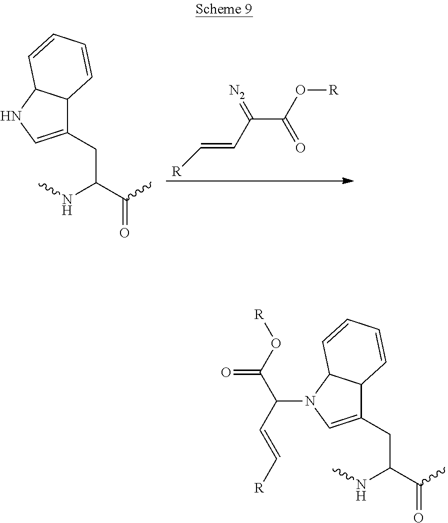

The present disclosure also provides a range of compositions, systems, and methods for labeling post-translationally modified amino acids. Non-limiting examples of post-translationally modified amino acids which may labeled included phosphorylated amino acids, glycosylated amino acids, nitrosylated amino acids, citrullinatinated amino acids, sulfenylated amino acids, and trimethylated amino acids. For example, as illustrated in Scheme 10, a phosphoserine or a phosphothreonine may also be selectively labeled via phosphoryl beta-elimination followed by label conjugate addition.

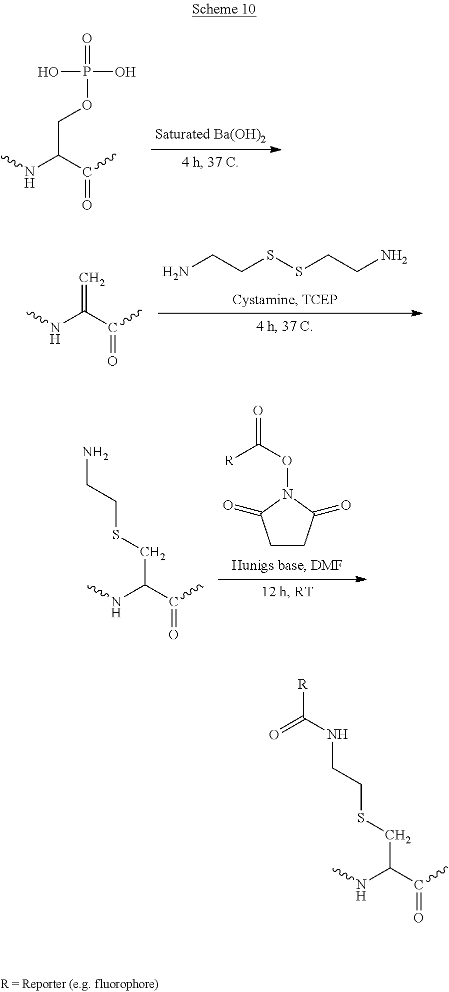

### Fluorosequencing

Various aspects of the present disclosure provide compositions, systems, and methods for peptide fluorosequencing. A fluorosequencing method disclosed herein can provide peptide sequence information at the single molecule level. For example, a fluorosequencing method may be used to identify a sequence of a peptide barcode, or to simultaneously determine sequences for a plurality of peptide barcodes. Exemplary fluorosequencing methods are provided in U.S. Pat. No. 9,625,469, U.S. patent application Ser. No. 16/709,903, and U.S. patent application Ser. No. 15/510,962). A method consistent with the present disclosure may subject a peptide to fluorosequencing and an additional form of analysis.

A characteristic feature of many fluorosequencing methods is coupling amino acid labels to a peptide to be sequenced. A label may be an amino acid specific label (e.g., configured to couple to a specific type of amino acid or a specific set of types of amino acids). A fluorosequencing method may comprise labeling a plurality of types of amino acids with separate, amino acid type specific labels. A fluorosequencing method may comprise labeling one, two, three, four, five, six, or more different types of amino acids residues in a subject peptide or protein. A peptide may comprise a label on an N-terminal amino acid, cysteine, lysine, glutamic acid, aspartic acid, tryptophan, tyrosine, serine, threonine, arginine, histidine, methionine, or any combination thereof. A peptide may comprise a label on a non-canonical amino acid, such as a phosphoserine/phosphothreonine, pyroglutamic acid, hydroxyproline, azidolysine, dehydroalanine, or any combination thereof. Each of these amino acid residues may be labeled with a different label. Multiple amino acid residues may be labeled with the same label such as (i) aspartic acid and glutamic acid or (ii) serine and threonine.

A label may comprise a reporter moiety. The reporter moiety may be optically detectable (e.g., fluorescent, phosphorescent, luminescent, or light absorbing). The reporter moiety may be electrochemically detectable (e.g., a redox active moiety with a characteristic oxidation or reduction potential). The reporter moiety may comprise a mass tag (e.g., for identification with mass spectrometry. A reporter moiety may identify a label to which it is attached. A plurality of labels may comprise a plurality of detectable moieties which identify labels of the plurality of labels by their type. For example, a method may comprise a plurality of types of labels configured to couple to different amino acids, each comprising a different reporter moiety that uniquely identifies the label by its type.

A label may comprise a reactive group. The reactive group may be configured to couple to a reporter moiety, a protecting group, or any combination thereof. A method may comprise coupling a label to an amino acid of a peptide (e.g., coupling a label to each amino acid of a particular type), and then coupling a reporter moiety or protecting group to the label. A method may comprise coupling a plurality of types of labels comprising reactive groups to a plurality of amino acids of a peptide, and coupling a plurality of reporter moieties, protecting groups, or combinations thereof to the labels based on their types. A method may comprise coupling a plurality of types of labels to a plurality of amino acids of a peptide, wherein the plurality of types of labels comprise labels with reactive groups, labels with reporter moieties (e.g., a cysteine-reactive label coupled to a dye), labels lacking reactive groups and reporter moieties, or any combination thereof.

A label (e.g., a label comprising a reactive group configured to couple to a reporter moiety or protecting group) may reversibly or irreversibly bind to an amino acid type, and thus may be chemically (e.g., by addition of a cleavage reagent) or physically (e.g., by addition of heat or light) decoupled from a target peptide. A method may thus comprise blocking a first amino acid, labeling a second amino acid type (e.g., threonine), unblocking the first amino acid type, and labeling the first amino acid type. Examples of reversible labels include can include silanes (e.g., trimethylsilane), acetyl groups, benzoyl groups, unsaturated pyran and furan groups, urea-forming groups, carbamate-forming groups, carbonate-forming groups, thiourea-forming groups, thiocarbamate-forming groups, thiocarbonate-forming groups, and derivatives thereof. Examples of irreversible labels can include alkyl groups, oxo-groups, amide-forming groups (e.g., an acyl chloride configured to convert an amine into an amide), and derivatives thereof.

Labeling specificity can be a major challenge for a fluorosequencing method. In many cases, a label may comprise reactivity toward a plurality of amino acid types. For example, some maleimide labels can react with cysteine, lysine, and N-terminal amines. A number of strategies may be employed to utilize or prevent such cross-reactivity. A method may comprise sequential amino acid labeling, for example to ensure that a multi-specific label is added to a system after one or more amino acid types with which the multi-specific label is configured to couple are chemically blocked or labeled, and therefore unable to react with the multi-specific label.

Fluorosequencing may comprise removing peptides through techniques such as chemical cleavage, Edman degradation, or other forms of enzymatic cleavage following or preceding subject peptide detection. Sequential peptide removal may generate sequence or position-specific information. For example, a reduction in fluorescence following an N-terminal amino acid removal step may indicate that a labeled amino acid, and thus that a specific type of amino acid, was disposed at a peptide N-terminal. Removal of each amino acid residue can be carried out with a variety of different techniques including Edman degradation and proteolytic cleavage. The techniques may include using Edman degradation to remove the terminal amino acid residue. Alternatively, the techniques may involve using an enzyme to remove the terminal amino acid residue. These terminal amino acid residues may be removed from either the C-terminus or the N-terminus of the peptide chain. In situations where Edman degradation is used, the amino acid residue at the N-terminus of the peptide chain is removed.

A label, reporter moiety, or protecting group of the present disclosure may be configured to withstand conditions for removing one or more of amino acid residues from a peptide. Some non-limiting examples of potential reporter moieties that may be used in the instant methods include, for example, those which emit a fluorescence signal in the red to infrared spectra such as an Alexa Fluor® dye, an Atto dye, Janelia Fluor® dye, a rhodamine dye, or other similar dyes. Examples of each of these dyes which were capable of withstanding the conditions of removing the amino acid residues include Alexa Fluor® 405, Rhodamine B, tetramethyl rhodamine, Janelia Fluor® 549, Alexa Fluor® 555, Atto647N, and (5)6-napthofluorescein. A reporter moiety may comprise fluorescent peptide (e.g., green fluorescent protein or a variant thereof) or an optically detectable material, such as a carbon nanotube, a nanorod, or a quantum dot.

Peptide detection or imaging may comprise immobilizing the peptide on a surface. The peptide may be immobilized to the surface by coupling a peptide-derived cysteine residue, the peptide N-terminus, or the peptide C-terminus with the surface or with a reagent coupled to the surface. The peptide may be immobilized by reacting the cysteine residue with the surface or with a capture reagent coupled to the surface. The peptide may be immobilized by coupling the peptide C-terminus or N-terminus with a capture moiety described herein. The peptide may be immobilized on a surface. Detecting the immobilized peptide may comprise capturing an image comprising the peptide. The image may comprise a spatial address specific to the peptide. A plurality of peptides may be detected in a single imagine, wherein one or more of the peptides may comprise a spatial address within the image. The surface may be optically transparent across the visible spectrum and/or the infrared spectrum. The surface may possess a low refractive index (e.g., a refractive index between 1.3 and 1.6). The surface may be between 10 to 50 nm thick, between 20 and 80 nm thick, between 50 and 200 nm thick, between 100 and 500 nm thick, between 200 and 800 nm thick, between 500 nm and 1 μm thick, between 1 and 5 μm thick, between 2 and 10 μm thick, between 5 and 20 μm thick, between 20 and 50 μm thick, between 50 and 200 μm thick, between 200 and 500 μm thick, or greater than 500 μm in thickness. The surface may be chemically resistant to organic solvents. The surface may be chemically resistant to strong acids such as trifluoroacetic acid or sulfuric acid. A large range of substrates (like fluoropolymers (Teflon-AF (Dupont), Cytop® (Asahi Glass, Japan)), aromatic polymers (polyxylenes (Parylene, Kisco, Calif.), polystyrene, polymethmethylacrytate) and metal surfaces (Gold coating)), coating schemes (spin-coating, dip-coating, electron beam deposition for metals, thermal vapor deposition and plasma enhanced chemical vapor deposition) and functionalization methodologies (polyallylamine grafting, use of ammonia gas in PECVD, doping of long chain end-functionalized fluoroalkanes etc.) may be used in the methods described herein as a useful surface. A 20 nm thick, optically transparent fluoropolymer surface made of Cytop® may be used in the methods described herein. The surfaces used herein may be further derivatized with a variety of fluoroalkanes that will sequester peptides for sequencing and modified targets for selection. Alternatively, an aminosilane modified surfaces may be used in the methods described herein. The methods may comprise immobilizing the peptides on the surface of beads, resins, gels, quartz particles, glass beads, or combinations thereof. In some non-limiting examples, the methods contemplate using peptides that have been immobilized on the surface of Tentagel® beads, Tentagel® resins, or other similar beads or resins. The surface used herein may be coated with a polymer, such as polyethylene glycol. The surface may be amine functionalized or thiol functionalized.

A sequencing technique described herein may involve imaging the peptide or protein to determine the presence of one or more labels or reporter moieties (e.g., amino acid labels) coupled to the peptide. The sequencing technique may comprise imaging a plurality of peptides or proteins to determine the presence of one or more labels or reporter moieties on individual peptides from among the plurality of peptides. The sequencing technique may comprise imaging at least 10³, at least 10⁴, at least 10⁵, at least 10⁶, at least 10⁷, at least 10⁸ or more proteins or peptides (e.g., imaging a portion of a surface comprising at least 10³ to at least 10⁸ proteins or peptides). These images may be taken after each removal of an amino acid residue and thus may enable determination of the location of the specific amino acid in the peptide sequence. For example, a C-terminal immobilized peptide may comprise a sequence (from N-terminal to C-terminal) of KDDYAGGGAAGKDA (SEQ ID NO: 2, wherein ‘K’ denotes lysine, ‘D’ denotes aspartate, ‘Y’ denotes tyrosine, ‘A’ denotes alanine, and ‘G’ denotes glycine), and may comprise labels coupled to each lysine and tyrosine residue. A first image comprising the C-terminal immobilized peptide may indicate the presence of two lysines and one tyrosine in the peptide. The N-terminal amino acid may be removed (e.g., by Edman degradation), such that a second image comprising the C-terminal immobilized peptide may indicate the presence of one lysine and one tyrosine in the peptide. This process may be repeated until a sequence of KXXYXXXXXXXKX (SEQ ID NO: 3) is identified for the peptide, wherein ‘X’ indicates a non-lysine, non-tyrosine amino acid, ‘K’ indicates a lysine, and ‘Y’ indicates a tyrosine. A method of the present disclosure can identify the position of a specific amino acid in a peptide sequence. A method may be used to determine the locations of specific amino acid residues in the peptide sequence or these results may be used to determine the entire list of amino acid residues in the peptide sequence. A method may involve determining the location of one or more amino acid residues in the peptide sequence and comparing these locations to known peptide sequences, which may identify the entire list of amino acid residues in the peptide sequence. For example, identifying the positions of the lysines and cysteines in a 40 amino acid fragment of a human protein may uniquely identify the protein (e.g., only one human protin contains the specific pattern of lysine and cysteine residues identified in the 40 amino acid fragment).

An imaging method may involve a variety of different spectrophotometric and microscopy methods, such as fluorimetry, diffuse reflectance, interferometric scattering, Raman, resonance enhanced Raman, infrared absorbance, visible light absorbance, ultraviolet absorbance, and fluorescence. The fluorescent methods may employ such fluorescent techniques, such as fluorescence polarization, Forster resonance energy transfer (FRET), or time-resolved fluorescence. A spectrophotometric or microscopy method may be used to determine the presence of one or more fluorophores coupled to a single peptide. Such imaging methods may be used to determine the presence or absence of a label on a specific peptide sequence. After repeated cycles of removing an amino acid residue and imaging a subject peptide, the position of the labeled amino acid residue can be determined in the peptide.

### Peptide Degradation

The present disclosure provides a range of chemical and enzymatic techniques for mild and sequential protein degradation. Degradation can be utilized in a range of peptide sequencing and analysis methods, for example to determine the order or identity of particular amino acids in a fluorosequencing assay. A peptide or protein may be iteratively subjected to cleavage conditions to determine the sequence of at least a portion of its sequence. The entire sequence of a peptide may be determined using the methods and compositions described herein. Controlled amino acid removal (e.g., N- or C-terminal amino acid removal) may be carried out through a variety of techniques including, for example, Edman degradation, organophosphate degradation, or proteolytic cleavage. In some instances, Edman degradation is used to remove a single terminal amino acid residue from a peptide N- or C-terminus. In some instances, the N-terminal amino acid residue is selectively removed from a peptide. A chemical or enzymatic technique for removing a terminal amino acid may remove a defined number of (e.g., exactly one, exactly two, at most two) amino acids. Accordingly, a method for analyzing a peptide may comprise successive degradation and analysis steps, such that the removal of a defined number of amino acids from an N-terminus or C-terminus per step provides position and sequence specific amino acid identifications during analysis. A chemical or enzymatic technique for removing a terminal amino acid may cleave a peptide at a defined location (e.g., only in between two alanine residues, or only at the peptide bond connecting an N-terminal amino acid to the remainder of a peptide).

An Edman degradation method may comprise chemically functionalizing a peptide N-terminus or C-terminus (e.g., to form a thiourea or a guanidinium derivative of an N-terminal amine), and then contacting the functionalized terminal amino acid with a reagent (e.g., a hydrazine), a condition (e.g., a high or low pH or temperature), or an enzyme (e.g., an Edmanase with specificity for the functionalized terminal amino acid) to remove the functionalized terminal amino acid.

A diactivated phosphate or phosphonate may be used for peptide cleavage. Such a method may utilize an acid to remove a functionalized amino acid. The diactivated phosphate or phosphonate may be a dihalophosphate ester. In other embodiments, the techniques involve using an enzyme to remove the terminal amino acid residue, such as, for example, an exopeptidase or an Edmanase. For example, a method may comprise derivatizing an N-terminal amino acid of a peptide with a diactivated phosphate, and contacting the peptide with an Edmanase with cleavage activity toward phosphate-functionalized N-terminal amino acids.

A cleavage method (e.g., a cleavage method implemented within a sequencing method) may comprise enzymatic cleavage. The cleavage method may comprise the use of a single protease, a series of proteases (e.g., provided in a specific order), or a combination of proteases. Exemplary proteases and their associated cleavage sites are provided in TABLE 1. A cleavage method may comprise decoupling a peptide barcode from a molecule. For example, a peptide barcode may comprise a cleavable linker comprising a cleavage site recognized by a protease listed in TABLE 1. In such cases, the sequence of the cleavage site may be present in the cleavable linker and absent in the peptide barcode. A cleavage method may comprise fragmenting a peptide barcode (e.g., cleaving an internal peptide bond prior to peptide barcode sequencing).

TABLE 1
Exemplary Proteases

| Protease | Cleavage Site |
|---|---|
| Carboxypeptidase A | C-terminal exopeptidase |
| Carboxypeptidase B | C-terminal exopeptidase; specific for lysine |
| | or arginine |
| Carboxypeptidase P | C-terminal exopeptidase |
| Carboxypeptidase Y | C-terminal exopeptidase |
| Cathepsin C | N-terminal exopeptidase; removes N-terminal |
| | dipeptide (except when N-terminal amino acid |
| | is lysine or arginine, or when 2^{nd} or 3^{rd} amino |
| | acid from N-terminal is proline) |
| Chymotrypsin | C-terminal exopeptidase; specific for |
| | phenylalanine, tryptophan, and tyrosine |
| Clostripain | arginine |
| Elastase | Alanine, Valine, Serine, Glycine, Leucine, or |
| | Isoleucine |
| Endoproteinase Arg-C | Arginine |
| Endoproteinase Glu-C | Glutamic Acid |
| Endoproteinase Lys-C | Lysine |
| Glutamyl endopeptidase | Glutamic acid |
| Kallikrein (Plasma) | Lysine or Arginine |
| Papain | Lysine or Arginine followed by a hydrophobic |
| | residue |
| Pepsin | Leucine, Phenylalanine, Tryptophan or |
| | Tyrosine |
| Proteinase K | Aliphatic and aromatic amino acids |
| Subtilisin | Hydrophobic amino acids |
| TEV Protease | Specific for the sequences Glutamic acid- |
| | Asparagine-Leucine-Tyrosine-Phenylalanine- |
| | Glutamine-Glycine and Glutamic acid- |
| | Asparagine-Leucine-Tyrosine-Phenylalanine- |
| | Glutamine-Serine; cleaves between Glutamine- |
| | Glycine or Glutamine-Serine |
| Thermolysin | Isoleucine, Methionine, Phenylalanine, |
| | Tryptophan, Tyrosine, or Valine |
| Trypsin | Lysine or Arginine |

Peptide cleavage may comprise chemical cleavage. Examples of chemical cleavage reagents consistent with the present disclosure include cyanogen bromide, BNPS-skatole, formic acid, hydroxylamine, and 2-nitro-5-thiocyanobenzoic acid. A peptide barcode may comprise a chemically cleavable moiety, such as a disulfide. A peptide barcode may be coupled to a molecule by a linker which comprises a chemically cleavable moiety. A peptide barcode may be coupled to a molecule by a chemically cleavable bond. A cleavage method may comprise a combination (e.g., parallel or sequential use) of chemical and enzymatic cleavage reagents. A cleavage method may comprise activating (e.g., functionalizing) an amino acid for chemical or enzymatic cleavage. For example, a method may comprise derivatizing an N-terminal amino acid residue of a peptide, and then contacting the peptide with an ‘Edmanase’ enzyme configured to remove the derivatized N-terminal amino acid residue.

Peptide cleavage conditions may be achieved with a solvent. The solvent may be an aqueous solvent, an organic solvent, or a combination or mixture thereof. The solvent may be an organic solvent. The organic solvent may comprise a miscibility with water. The organic solvent may be anhydrous. The solvent may be a non-polar solvent (e.g., hexane, dichloromethane (DCM), diethyl ether, etc.), a polar aprotic solvent (e.g., tetrahydrofuran (THF), ethyl acetate, dimethylformamide (DMF), acetonitrile (MeCN), dimethyl sulfoxide (DMSO), etc.), or a polar protic solvent (e.g., isopropanol (IPA), ethanol, methanol, acetic acid, water, etc.). The solvent may be DMF. The solvent may be a C₁-C₁₂ haloalkane. The C₁-C₁₂ haloalkane may be DCM. The solvent may be a mixture of two or more solvents. The mixture of two or more solvents may be a mixture of a polar aprotic solvent and a C₁-C₁₂ haloalkane. The mixture of two or more solvents may be a mixture of DMF and DCM. The mixture of solvents may be any combination thereof.

A degradation process may comprise a plurality of steps. For example, a method may comprise an initial step for derivatizing a terminal amino acid of a peptide, and a subsequent step for cleaving the derivatized terminal amino acid from the peptide. One such method comprises organophosphorus compound-mediated N-terminal functionalization and removal, and thus provides an alternative to the isothiocyanate (e.g., phenyl isothiocyanate) based processes of some Edman degradation schemes.

An organophosphate-based degradation scheme may comprise dissolving a peptide in an organic solvent or organic solvent mixture (e.g., a mixture of dichloromethane and dimethylformamide) in the presence of an organic base (e.g., triethylamine, N, N-diisopropylethylamine (DIPEA), 1,8-diazabicyclo[5.4.0]undec-7-ene (DBU), pyridine, 1,5-diazabicyclo(4.3.0)non-5-ene, 2,6-di-tert-butylpyridine, imidazole, histidine, sodium carbonate, etc.). The peptide may then be contacted with at least one organophosphorus compound. The cleavage of the peptide or protein N-terminus may be initiated through the addition of a weak acid (e.g., formic acid in water). The cleavage of the peptide or protein N-terminus may also be initiated with water. The resulting products may include the terminal amino acid of the peptide or protein released from the peptide as a phosphoramide and the peptide or protein that is shortened by the terminal amino acid residue, which comprises a free N-terminus that can be used to perform a subsequent cleavage reaction.

A cleavage method may comprise digesting a peptide to generate fragments of a desired average length. The cleavage method may generate peptides (e.g., by acting upon a complex mixture of peptides, such as cell lysate) with an average length of at least 5 amino acids, at least 8 amino acids, at least 10 amino acids, at least 12 amino acids, at least 15 amino acids, at least 20 amino acids, at least 25 amino acids, at least 30 amino acids, at least 40 amino acids, or at least 50 amino acids. The cleavage method may generate peptides with an average length of at most 50 amino acids, at most 40 amino acids, at most 30 amino acids, at most 25 amino acids, at most 20 amino acids, at most 15 amino acids, at most 12 amino acids, at most 10 amino acids, at most 8 amino acids, or at most 5 amino acids. The cleavage method may generate peptide fragments with an average length of between 5 and 20 amino acids, between 5 and 30 amino acids, between 10 and 20 amino acids, between 10 and 30 amino acids, between 12 and 18 amino acids, between 15 and 30 amino acids, between 20 and 40 amino acids, or between 30 and 50 amino acids.

A reaction mixture may comprise a stoichiometric or an excess concentration of a cleavage compound (e.g., relative to the concentration of peptides to be cleaved). The reaction mixture may comprise at least about 0.001% v/v, about 0.01% v/v, about 0.1% v/v, about 1% v/v, about 5% v/v, about 10% v/v, about 15% v/v, about 20% v/v, about 30% v/v, about 40% v/v, about 50% v/v, or more of the cleavage compound. The reaction mixture may comprise at most about 50% v/v, about 40% v/v, about 30% v/v, about 20% v/v, about 15% v/v, about 10% v/v, about 5% v/v, about 1% v/v, about 0.1% v/v, about 0.01% v/v, about 0.001% v/v, or less of the cleavage compound. The reaction mixture may comprise from about 0.1% v/v to about 20% v/v, about 0.5% v/v to about 10% v/v, or about 1% v/v to about 10% v/v of the cleavage compound. The reaction mixture may comprise about 5% v/v of the cleavage compound.

The reaction may be performed at a temperature of at least about 0° C., at least about 5° C., at least about 10° C., at least about 15° C., at least about 20° C., at least about 25° C., at least about 30° C., at least about 40° C., at least about 50° C., at least about 60° C., at least about 70° C., at least about 80° C., or at least about 90° C. The reaction may be performed at a temperature of at most about 90° C., at most about 80° C., at most about 70° C., about 60° C., about 50° C., about 40° C., about 30° C., about 25° C., about 20° C., about 15° C., about 10° C., about 5° C., about 0° C., or less. The reaction may be performed at a temperature from about 0° C. to about 70° C., about 10° C. to about 50° C., about 20° C. to about 40° C., or about 20° C. to about 30° C. The reaction may be performed at a temperature above room temperature (e.g., about 22° C. to about 27° C.). The reaction may be performed at room temperature. The reaction may be performed at close to 0° C. or below 0° C. (e.g., in the presence of an antifreeze).

The peptide and the cleavage compound may be mixed or incubated for at least about 1 minute, about 5 minutes, about 10 minutes, about 20 minutes, about 30 minutes, about 40 minutes, about 50 minutes, about 60 minutes, about 2 hours, about 3 hours, about 4 hours, about 6 hours, about 8 hours, about 10 hours, about 12 hours, about 16 hours, about 20 hours, about 24 hours, or more. The peptide and the cleavage compound may be mixed or incubated for at most about 24 hours, about 20 hours, about 16 hours, about 12 hours, about 10 hours, about 8 hours, about 6 hours, about 4 hours, about 3 hours, about 2 hours, about 1 hour, about 50 minutes, about 40 minutes, about 30 minutes, about 20 minutes, about 10 minutes, about 5 minutes, about 1 minute, or less. The peptide and the cleavage compound may be mixed or incubated from about 1 minute to about 24 hours, 5 minutes to about 6 hours, 5 minutes to about 2 hours, or 5 minutes to about 30 minutes.

### Labels With Multiple Reactive Groups

Aspects of the present disclosure provide amino acid labels comprising a first reactive group for coupling to an amino acid (or a portion thereof, such as a reactive functional group of an amino acid side chain) and a second reactive group for coupling to a reporter moiety or a protecting group. Such a system may be referred to as a “click-clack” labeling system, wherein a “click” reagent refers to a label configured to couple to an amino acid, and a “clack” reagent refers to a reporter moiety or protecting group configured to couple to the “click” reagent. The second reactive group of a label may be configured to reversibly or irreversibly couple to a reporter moiety, a protecting group, or any combination thereof. The second reactive group may be reversibly coupled to a protecting group, decoupled from the protecting group, and then coupled to a reporter moiety. For example, the label may be provided with a protecting group coupled to its first or second reactive group (e.g., a diol coupled to an aldehyde reactive group of the label). Such a modular labeling process may enable multi-amino acid labeling schemes with diminished cross-reactivity between amino acid and label types. Such a labeling process may also enable the use of chemically sensitive reporter moieties (e.g., pH sensitive or chemically quenchable dyes), by allowing their attachment following amino acid labeling steps. For example, a method may comprise selectively labeling cysteine residues of a peptide with a first label, selectively labeling lysine residues of the peptide with a second label, selectively labeling carboxylate-containing residues (e.g., aspartate and glutamate) of the peptide with a third label, selectively labeling arginine residues of the peptide with a fourth label, chemically modifying (e.g., oxidizing) methionine residues of the peptide, selectively labeling the chemically modified methionine residues of the peptide with a fifth label, and coupling different reporter moieties (e.g., different color dyes) to each of the first, second, third, fourth, and fifth labels in a single step (e.g., upon addition of all labeling reagents simultaneously). It is also conceivable that one or more reporter fluorophores would directly label the amino acids on the peptide chains. A bifunctional label of the present disclosure may prevent cross-reactivity between a first reactive group of a label and a reporter moiety. For example, the use of bifunctional labels may permit use of reporter moieties which are cross-reactive with a first reactive group of a label, such as an iodoacetamide-reactive dye and a label comprising a cysteine reactive iodoacetamide group.

A label of the present disclosure may be used to crosslink two biological species, such as two amino acid residues. For example, a method may comprise coupling a lysine selective label to a first peptide and a cysteine selective label to a second peptide, and then cross-linking the lysine and cysteine selective labels. The cross-linking may directly couple (e.g., through a chemical bond) the lysine and cysteine selective labels, or may comprise a linker, such as a “clack” reagent configured to couple to second reactive groups on the lysine and cysteine selective labels.

Examples of amino acid selective labels comprising second reactive groups, as well as example reagent pairs for their syntheses, are provided in TABLE 2. A cysteine- and lysine-selective “Click” label may comprise an iodoacetamide as a first reactive group (e.g., for coupling to cysteine or lysine) and an azide as a second reactive group (e.g., for coupling to a “Clack” reporter moiety or protecting group), such as the iodoacetamide PEG azide compound shown in Row A of TABLE 2. A cysteine-selective “Click” label may comprise an iodoacetamide as a first reactive group and a norbornene as a second reactive group, such as the reactant shown in Row B of TABLE 2. Such a reagent may be synthesized by coupling a norbornene amine with an iodoacetamide N-hydroxysuccinamide ester. A cysteine-selective “Click” label may comprise an iodoacetamide as a first reactive group and an aldehyde as a second reactive group, such as 2-iodo-N-(3-oxopropyl)acetamide (as shown in Row C of TABLE 2). Such a compound may be generated by coupling an N-hydroxysuccinamide ester with an amine comprising a geminal diether configured to hydrolyze to an aldehyde. A cysteine-selective label may comprise a first reactive group for coupling to cysteine but lack a second reactive group (e.g., the label may be a “dummy” label), and therefore be unable to couple to a “Clack” reporter moiety or protecting group) reagent. An example of such a reagent may be iodoacetamide, as shown in TABLE 2 Row D.

A lysine-selective “Click” label may comprise an N-hydroxysuccinamide ester as a first reactive group and a norbornene as a second reactive group, such as the reagent shown in Row F of TABLE 2. A lysine-selective “Click” label may comprise an N-hydroxysuccinamide ester as a first reactive group and a geminal diether as a second reactive group, such as the reagent shown in Row G of TABLE 2. Such a reagent may be generated by coupling 1-hydroxypyrrolidine-2,5-dione to the carboxylic acid of a compound comprising a geminal diether. A lysine-selective label may comprise a first reactive group for coupling to lysine but lack a second reactive group for coupling to a “Clack” reporter moiety or protecting group. An example of such a reagent may be an activated ester, such as the compound shown in Row H of TABLE 2.

A carboxylate-selective (e.g., selective for aspartate and glutamate side chain carboxylates) “Click” label may comprise an amine as a first reactive group and an azide as a second reactive group, such as the reagent shown in Row I of TABLE 2. A carboxylate-selective “Click” label may comprise an amine as a first reactive group a norbornene as a second reactive group, such as the reagent shown in Row J of TABLE 2. A carboxylate-selective “Click” label may comprise an amine as a first reactive group a geminal diether as a second reactive group such as the reagent shown in Rows K and L of TABLE 2. A carboxylate-selective label may comprise a first reactive group for coupling to a carboxylate but lack a second reactive group for coupling to a “Clack” reporter moiety or protecting group. An example of such a reagent may be an alkyl amine, such as the compound shown in Row M of TABLE 2.

A phosphoserine-, phosphothreonine-, and/or glycosylation-selective “Click” reagent may comprise a disulfide as a first reactive group and an azide, a norbornene, a geminal diether, or an aldehyde as a second reactive group, as shown in Rows N-R of TABLE 2. A phosphoserine-, phosphothreonine-, and/or glycosylation-selective “Click” reagent may comprise a disulfide as a first reactive group and may lack a second reactive group.

TABLE 2
Exemplary ″Click″ Labels Consistent With The Present Disclosure

| ″CLICK″ | | | |
|---|---|---|---|
| LABEL | | | COMMERCIAL AVAILABILITY & |
| TYPE | ROW | ″CLICK″ LABEL | REAGENTS FOR ″CLICK″ LABEL SYNTHESIS |
| CYSTEINE ″CLICK″ LABELS | A | 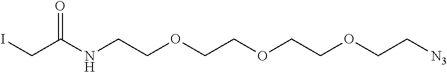 [SMILES: [N-]=[N+]=NCCOCCOCCOCCNC(=O)CI] | COMMERCIALLY AVAILABLE |
| | B | 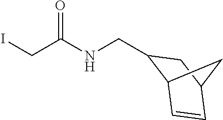 [SMILES: O=C(CI)NCC1CC2C=CC1C2] | 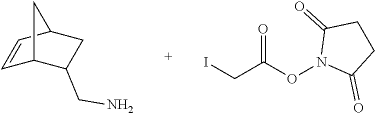 [SMILES: NCC1CC2C=CC1C2 ; O=C(CI)ON1C(=O)CCC1=O] |
| | C | 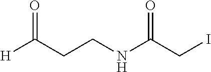 | 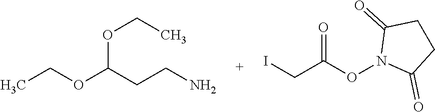 NH- |
| | D | 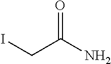 | COMMERCIALLY AVAILABLE |
| LYSINE ″CLICK″ LABELS | E | 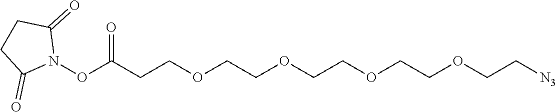 [SMILES: [N-]=[N+]=NCCOCCOCCOCCOCCC(=O)ON1C(=O)CCC1=O] | COMMERCIALLY AVAILABLE |
| | F | 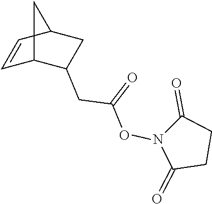 [SMILES: O=C(CC1CC2C=CC1C2)ON1C(=O)CCC1=O] | COMMERCIALLY AVAILABLE |
| | G | 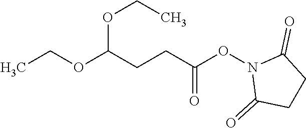 [SMILES: CCOC(CCC(=O)ON1C(=O)CCC1=O)OCC] | 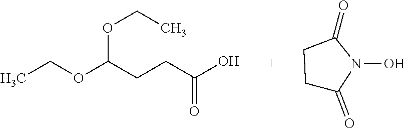 |
| | H | 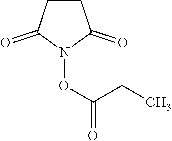 | COMMERCIALLY AVAILABLE |
| CARBOXYLATE ″CLICK″ LABELS | I | 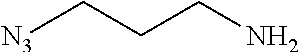 | COMMERCIALLY AVAILABLE |
| | J | 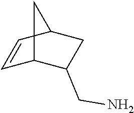 | COMMERCIALLY AVAILABLE |
| | K | 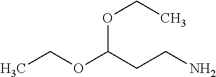 | COMMERCIALLY AVAILABLE |
| | L | | COMMERCIALLY AVAILABLE |
| | M | 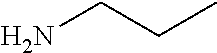 | COMMERCIALLY AVAILABLE |
| PHOSPHOSERINE, PHOSPHOTHREONINE, & | N | 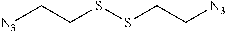 [SMILES: [N-]=[N+]=NCCSSCCN=[N+]=[N-]] | COMMERCIALLY AVAILABLE |
| GLYCOSYLATION | | | |
| ″CLICK″ | | | |
| LABELS | | | |
| | O | 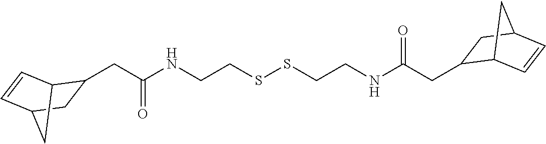 [SMILES: O=C(CC1CC2C=CC1C2)NCCSSCCNC(=O)CC1CC2C=CC1C2] | 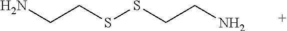 |
| | | | 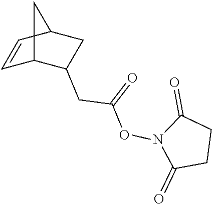 [SMILES: O=C(CC1CC2C=CC1C2)ON1C(=O)CCC1=O] |
| | P | 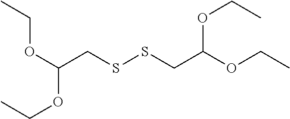 [SMILES: CCOC(CSSCC(OCC)OCC)OCC] | COMMERCIALLY AVAILABLE |
| | Q | 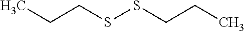 | COMMERCIALLY AVAILABLE |
| | R | 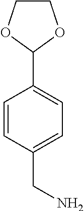 [SMILES: NCc1ccc(C2OCCO2)cc1] | COMMERCIALLY AVAILABLE |

Sample preparation may be improved by labeling a plurality of amino acid residues through series of sequential steps. The present disclosure provides a range of systems to facilitate labeling of multiple amino types. The system may minimize cross-reactivity of amino acids, reporter moieties (e.g., fluorescent molecules (e.g., dyes)), or the decomposition of, for example, sensitive reporter moieties (e.g., fluorescent molecules (e.g., dyes)).

In another aspect, provided herein is a system comprising a peptide, wherein said peptide: is immobilized to at least one support; and comprises an amino acid coupled to a label, wherein said label comprises (i) a first reactive group that is configured to couple to a second reactive group that is coupled to a reporter moiety configured to emit a signal or (ii) a protecting group configured to prevent coupling between said label and said second reactive group.

In another aspect, provided herein is a system for processing or analyzing a peptide, comprising a peptide comprising an amino acid coupled to a first reactive group and a support coupled to a second reactive group, wherein the first reactive group is configured to couple to the second reactive group to immobilize the peptide adjacent to the support. In some embodiments, the system is configured to couple a peptide to a support (e.g., a surface). In some embodiments, the system is configured to couple an amino acid residue of a peptide to a support (e.g., a surface). The support (e.g., the surface) may comprise a reactive group configured to couple to a functional group coupled to the amino acid residue of the peptide.

The peptide may comprise a plurality of amino acids. The peptide may be an oligomer or polymer comprising amino acids or amino acid analogues. The peptide may comprise amino acids that are L-amino acids or D-amino acids. A peptide may be synthetic, recombinant, or naturally occurring. A synthetic peptide may be a peptide that is produced by artificial approaches in vitro. At least one amino acid of the plurality of amino acids may be selected from the group consisting of lysine, cysteine, glutamic acid, aspartic acid, tyrosine, arginine, histidine, threonine, serine, glutamine, asparagine and tryptophan. The plurality of amino acids may comprise one or more amino acids, the one or more amino acid selected from the group consisting of lysine, cysteine, glutamic acid, aspartic acid, tyrosine, arginine, histidine, threonine, serine, glutamine, asparagine and tryptophan. The peptide may comprise one amino acid selected from the group consisting of lysine, cysteine, glutamic acid, aspartic acid, tyrosine, arginine, histidine, threonine, serine, glutamine, asparagine and tryptophan. The plurality of amino acids may comprise a non-natural amino acid. The plurality of amino acids may comprise a D-amino acid.

At least one amino acid of the plurality of amino acids may be coupled to a label. The plurality of amino acids may comprise at least two or more amino acid types. The at least two amino acid or more types may comprise a first amino acid type and a second amino acid type. The first amino acid type may be coupled to a first label. The second amino acid type may be coupled to a second label. The first amino acid type may be coupled to a first label and the second amino acid type may be coupled to a second label. The first label and the second label may each be coupled to a different reporter moiety. The plurality of amino acids may comprise at least one, two, three, four, five, six, seven, eight, nine, ten, eleven, or more amino acid types. The plurality of amino acids may comprise between two and twenty amino acid types. The plurality of amino acids may comprise between 4 and 18 amino acid types. The plurality of amino acids may comprise between 6 and 16 amino acid types. The plurality of amino acids may comprise between 8 and 14 amino acid types. The plurality of amino acids may comprise between 9 and 11 amino acid types. Less than all of the amino acid types of the plurality of amino acids may labelled. Each amino acid type of the at least two amino acid types may be coupled to a different label. The peptide may comprise at least four amino acid types, wherein each amino acid type of said at least four amino acid types are coupled to a different label. Less than all of the plurality of amino acids may be labelled. Each of the plurality of amino acids may be labelled.

The plurality of amino acids may comprise at least two amino acid types, and each amino acid type of the at least two amino acid types may be coupled to a different label. The peptide may comprise at least three amino acid types, wherein each amino acid type of said at least three amino acid types are coupled to a different label. The peptide may comprise at least four amino acid types, wherein each amino acid type of said at least four amino acid types are coupled to a different label. The peptide may comprise at least five or six amino acid types, wherein each amino acid type of said at least five or six amino acid types are coupled to a different label. The peptide may comprise at least eight amino acid types, wherein each amino acid type of said at least eight amino acid types are coupled to a different label. The peptide may comprise at least ten amino acid types, wherein each amino acid type of said at least ten amino acid types are coupled to a different label. Each label coupled to a different amino acid type may independently be coupled to a reporter moiety configured to emit a signal corresponding to each amino acid type. In some cases, the majority of the plurality of amino acids are labelled. In some cases, the majority of the plurality of amino acids are unlabeled.

The amino acid that can be coupled to a label may be an amino acid selected from the group consisting of lysine, cysteine, glutamic acid, aspartic acid, tyrosine, arginine, histidine, threonine, serine, proline, asparagine, glutamine, and tryptophan. The amino acid that is coupled to a label may comprise a post-translational modification. The post translational modification can be glycosylation, acetylation, alkylation, biotinylation, glutamylation, glycosylation, isoprenylation, phosphorylation, lipolation, phosphopantetheinylation, sulfation, selenation, amidation, ubiquitination, hydroxylation, nitration, nitrosylation, citrullination, cyclization (such as N-terminal glutamate or glutamine cyclization), and SUMOylation.

The peptide may comprise a plurality of amino acids coupled to a plurality of labels. The plurality of amino acids may comprise a plurality of amino acids coupled to a plurality of labels. The plurality of amino acids coupled to a plurality of labels may comprise a first amino acid coupled to a first label and a second amino acid coupled to a second label. The plurality of amino acids may comprise a plurality of first amino acids coupled to a plurality of first labels. The plurality of amino acids may comprise a plurality of second amino acids coupled to a plurality of second labels. The plurality of amino acids may comprise (i) a plurality of first amino acids coupled to a plurality of first labels and (ii) a plurality of second amino acids coupled to a plurality of second labels. The first label, or the plurality thereof, may couple only to the first amino acid, or the plurality thereof. The second label, or the plurality thereof, may couple only to the second amino acid, or the plurality thereof. The first label, or the plurality thereof, may couple only to the first amino acid, or the plurality thereof, and the second label, or the plurality thereof, couples only to the second amino acid, or the plurality thereof. At least one label of the plurality of labels may be coupled to a specific amino acid type of the plurality of amino acids. For example, one label of the plurality of labels may be coupled to a lysine, a cysteine, a glutamic acid, an aspartic acid, a tyrosine, an arginine, a histidine, a threonine, a serine, a glutamine, an asparagine, or a tryptophan.

A label may comprise a first reactive group that is configured to couple to a second reactive group. The first reactive group may be selected from the group consisting of an azide, an alkyne, an alkene, an aldehyde, a ketone, a tetrazine, a thiol, a dithiol, a cyclooctene, and norbornene. The second reactive group may be selected from the group consisting of an azide, an alkyne, an alkene, an aldehyde, a ketone, a tetrazine, a thiol, a dithiol, a cyclooctene, and norbornene. The first reactive group may be selected from the group consisting of an azide, an alkene, an aldehyde, a ketone, and a tetrazine. The first reactive group may be a strained alkyne. The second reactive group may be selected from the group consisting of an azide, a thiol, a dithiol, a cyclooctene, an alkene, an aldehyde, a ketone, a tetrazine, a norbornene, and an alkyne. The second reactive group may be a strained alkyne.

At least one label of the plurality of labels may be configured to react with a specific second reactive group coupled to a specific reporter moiety. The first reactive group may be selected from the group consisting of an alkyne, a thiol, a dithiol, and a cyclooctene, and the second reactive group may be selected from the group consisting of an alkyne, an azide, a thiol, a dithiol, a cyclooctene, an alkene, an aldehyde, a ketone, a tetrazine, and a norbornene. The first reactive group may be configured to react to a particular second reactive group. For example, the first reactive group may be an azide and the second reactive group may be an alkyne, the first reactive group may be an alkyne and the second reactive group may be an azide, the first reactive group may be an alkene and the second reactive group may be a thiol, the first reactive group may comprise an carbonyl (e.g., a ketone or an aldehyde) and the second reactive group may be an dithiol, the first reactive group may be a tetrazine and the second reactive group may be a cyclooctene (e.g., trans-cyclooctene).

The at least one label that couples to the amino acid, or plurality thereof, may be coupled to a reporter moiety. The reporter moiety may be configured to emit a signal upon excitation. The signal can be a detectable signal. For example, the signal can be an optical signal, such as a fluorescent or phosphorescent signal. The optical signal may be produced by a dye. The reporter moieties may also produce non-optically detectable signals. For example, a reporter moiety may produce an electrical signal, a radioactive signal or a chemical signal. The reporter moiety may be coupled to a spacer. The spacer may adjoin a reporter moiety and a second reactive group. A reporter may be configured to react with the label. The reporter may comprise a reporter moiety and a reactive group (e.g., a second reactive group). The reporter may comprise a reporter moiety, a reactive group (e.g., a second reactive group), and a spacer.

The at least one support may be a bead, a polymer matrix, an array, or any combination thereof. The at least one support may be a bead, a polymer matrix, or an array. The at least one support may be a bead and an array. The at least one support can be a bead. The at least one support can be an array. The array can be a surface. The array can be a slide. The slide can be a microscopic slide. The at least one support can be a microscopic slide. The at least one support can be a polymer matrix.

The support may be a solid support or a semi-solid support. The solid support or semi-solid support may be a bead. The bead may be a gel bead. The bead may be a polymer bead. The support may be a resin. Non-limiting supports may comprise, for example, agarose, sepharose, polystyrene, polyethylene glycol (PEG), or any combination thereof. The support may be a polystyrene bead. The support may include functional groups, such as, for example, amines, sulfhydryls, acids, alcohols, bromides, maleamides, succinimidyl esters (NHS), sulfosuccinimidyl esters, disulfides, azides, alkynes, isothiocyanates (ITC), or combinations thereof. The support may be a PEGA resin. The support may be an amino PEGA resin. The support may comprise an amine group. The support may include protected functional groups, such as, for example, Boc, Fmoc, alkyl ester, Cbz, or combinations thereof. The bead may contain a metal core. The bead may be a polymer magnetic bead. The polymer magnetic bead may comprise a metal-oxide. The support may comprise at least one iron oxide core.

An N-terminus, a C-terminus, an internal amino acid, or any combination thereof, of the peptide can be coupled to the at least one support. The N-terminus and the C-terminus of the peptide can be coupled to the at least one support. The N-terminus of the peptide may be coupled to one support and the C-terminus of the peptide can be coupled to another support. The N-terminus of the peptide may be coupled to a bead. The C-terminus of the peptide may be coupled to a slide. The N-terminus of the peptide may be coupled to a bead and the C-terminus may not be coupled to a support. The C-terminus of the peptide may be coupled to a slide and the N-terminus of the peptide may not be coupled to a support. The N-terminus of the peptide may be coupled to a bead and the C-terminus of the peptide may be coupled to a slide.

The N-terminus of the peptide can be coupled to the at least one support. The N-terminus of the peptide can be coupled to a cleavable unit. The cleavable unit can be coupled to the at least one support. The cleavable unit may comprise at least one of (i) a cleavable moiety, (ii) an aldehyde, (iii) said at least one support, or (iv) a spacer. The cleavable unit may comprise a cleavable moiety. The cleavable moiety may comprise a rink group. The cleavable unit may comprise an aldehyde. The aldehyde may be a pyridinecarboxaldehyde (PCA), or any derivative thereof. The cleavable unit may comprise a spacer. The cleavable unit may comprise at least two of (i) a cleavable moiety, (ii) an aldehyde, (iii) said at least one support, or (iv) a spacer. The cleavable unit may comprise a cleavable moiety and an aldehyde. The cleavable unit may comprise the at least one support and an aldehyde. The cleavable unit may comprise at least three of (i) a cleavable moiety, (ii) an aldehyde, (iii) said at least one support, or (iv) a spacer. The cleavable unit may comprise an aldehyde, the at least one support, and a spacer. The cleavable unit may comprise an aldehyde, the at least one support, and a cleavable moiety. The cleavable unit may comprise a spacer, the at least one support, and a cleavable moiety. The cleavable unit may comprise (i) a cleavable moiety, (ii) an aldehyde, (iii) said at least one support, and (iv) a spacer. The cleavable can be as described in WO2020072907A1. The aldehyde, the spacer, the cleavable moiety, or any combination thereof can be as described in WO2020072907A1.

The C-terminus of the peptide can be modified with an agent configured to couple the C-terminus to at least one support. The agent may comprise an alkyne or an azide, either of which may be configured to couple to at least one support. The C-terminus may comprise an acidic amino acid. The C-terminus may comprise a first acidic residue and a second acidic residue. The first acidic residue may be a C-terminal carboxylic acid. The second acidic residue may be an aspartic acid side chain or a glutamic acid side chain. The first acidic residue and second acidic residue of the C-terminus may be modified. In cases where the C-terminus of the peptide contains two acidic residues, both the first and second acidic residues may be modified by an agent comprising an alkyne or an azide, either of which may be configured to couple to at least one support.

A reporter (or a reporter moiety) for use in the present system may, by way of a non-limiting example, emit a detectable or an optical signal (e.g., from a fluorescent dye). However, any number of reporters (or a reporter moieties) as described herein may be used for their various advantageous features. As an additional example, a reporter (or a reporter moiety) may emit a radiometric signal, which could be detected by an ionization chamber, a gaseous ionization detector, a Geiger counter, a photodetector, a scintillation counter, or a semiconductor detector, among others. Conversely, a reporter (or a reporter moiety) may not emit a signal at all. Reporters (and reporter moieties) may selectively label specific amino acids by reacting with their side chains, or may detect a post-translational modification to an amino acid. In some examples, a plurality of amino acids will be contained within the peptide, of which many or all may be coupled to a label and/or a reporter (or a reporter moiety).

A peptide, composed of two or more amino acids, may have an N-terminus and a C-terminus. These termini may be separated by one or more amino acids. The N-terminus is a terminal amino acid and may contain a terminal amine. The terminal amine may be unsubstituted, or may be substituted. In some instances, the amine may be cleaved, blocked, functionalized, or otherwise modified. Naturally-occurring peptides generally contain an unsubstituted amine at the N-terminal position. Any amino acid can become an N-terminus following a bond cleaving event. Similarly, the C-terminus is a terminal amino acid and may contain a terminal carboxylic acid. The terminal carboxylic acid may be unsubstituted or substituted. In some instances, the carboxylic acid may be cleaved, blocked, functionalized, or otherwise modified. Naturally-occurring peptides generally contain an unsubstituted carboxylic acid at the C-terminal position. Any amino acid can become a C-terminus following a bond cleavage event. In some examples, as provided herein, the C-terminus may be any amino acid. In other examples, the C-terminus is an acidic amino acid (e.g., glutamate or aspartate). The present disclosure provides for specific cleavage of a first peptide at a known site in order to yield a second peptide with a specific (e.g., desired) C-terminal amino acid residue. The C-terminal amino acid, following cleavage, may be an acidic residue. The C-terminal amino acid, following cleavage, may be a non-acidic residue. Similarly, a first peptide can be intentionally cleaved to yield a second peptide with a specific (e.g., desired) N-terminal amino acid residue.

Peptides may have non-linear structures. In some cases, a peptide may be branched. In some cases, a peptide may be cyclic. In some cases, two or more peptides may be crosslinked. In some cases, two or more peptides may be covalently crosslinked. In some cases, two or more peptides may be non-covalently associated.

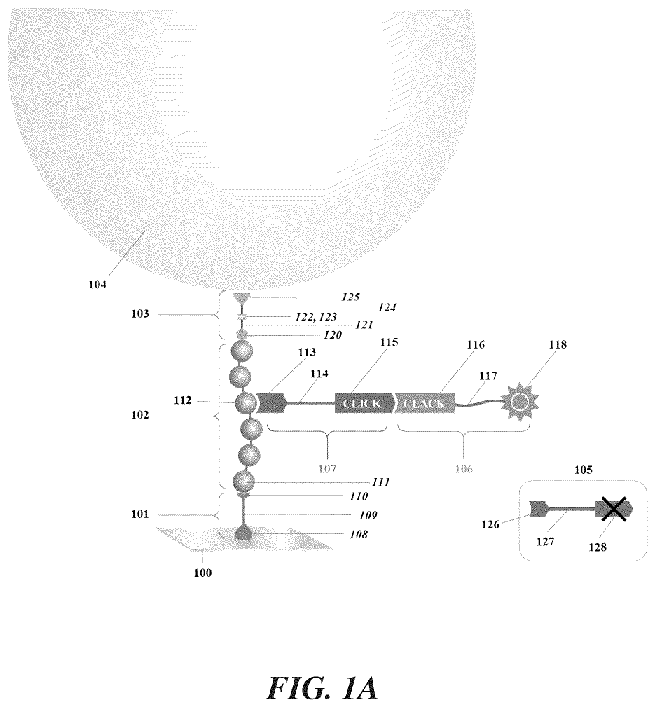

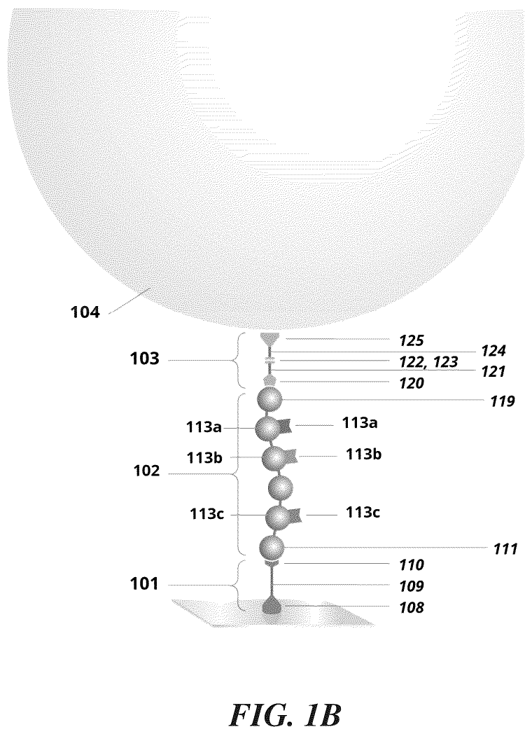

The system may be configured as shown in FIG. 1A. The system may comprise a peptide 100 comprising at least one amino acid (e.g., 111, 112, 119). The peptide may comprise an N-terminal amino acid 119, a C-terminal amino acid 111, and an internal amino acid 112. The N-terminal amino acid may be coupled to an N-terminal capture agent 103. The N-terminal capture agent may comprise a first reactive handle 120 for coupling to the N-terminal amino acid. The N-terminal capture agent comprise a second reactive handle 125 configured to couple to a support 104, such as a bead. The support 104 and first reactive handle 120 may be connected by a cleavable linker comprised of a first linking group 121, a cleavable linker 122 and 123, and a second linking group 124, such that cleavage of the cleavable linker 122 and 123 liberates the peptide 102 from the support 104. The C-terminal amino acid may be coupled to a surface attachment agent 101, which may comprise a reactive handle 110 for coupling to the peptide C-terminus 111, a linker 109, and a reactive handle 108 configured to couple to a surface 100. A label 107 may be coupled to at least one amino acid of the peptide 102 (e.g., an internal amino acid 112). The label may comprise a first reactive group 112 coupled to the amino acid 112, a linker 113, and a second reactive group 114. In the configuration shown, the second reactive group is coupled to a reactive group 116 of a reporter moiety 106 a detectable moiety 118, such as a fluorophore. The detectable moiety 118 may be directly coupled to the reactive group 116 or may be coupled to the reactive group by a linker 117. In alternative configurations, the second reactive group 115 of the label may be coupled to reactive group 126 of a protecting group 105. The protecting group may comprise a chemically inert moiety 128, which may be directly coupled to the reactive group 126, or may be coupled to the reactive group by a linker 127.

FIG. 1B provides a configuration of the system of FIG. 1A in which a plurality of labels 113a, 113b, and 113c are coupled to a plurality of internal amino acids 112a, 112b, 112c. In certain configurations, the plurality of labels comprise multiple types of labels coupled to different amino acids. In certain configurations, the plurality of labels are of a single type. The plurality of labels may be coupled to a plurality of reporter moieties, protecting groups, or a combination thereof.

### 3. Methods

In another aspect, provided herein is a method for improving sample preparation for analyzing a peptide. The method may improve sample preparation for peptide sequencing (e.g., fluorosequencing). Sample preparation may be improved by selectively labeling at least one amino acid side chain, including those of N-terminal amino acids, C-terminal amino acids, and internal amino acids. Sample preparation may be improved by efficiently labeling at least one amino acid side chain through quantitative reaction between a first reactive group and a second reactive group. Provided herein may be a method for labeling multiple amino acids or amino acid types. The method may minimize cross-reactivity of amino acids, reporter moieties (e.g., fluorescent molecules (e.g., dyes)), or the decomposition of, for example, sensitive reporter moieties (e.g., fluorescent molecules (e.g., dyes)).

In another aspect, provided herein is a method for labeling an amino acid of a peptide, comprising providing the peptide, wherein the peptide comprises an internal amino acid coupled to an azide and a C-terminus coupled to an alkyne; and bringing the peptide in contact with a first reporter under conditions such that the first reporter reacts with the internal amino acid. The first reporter may comprise an alkyne. The alkyne may be a strained alkyne. The conditions that allow the first reporter to react with the internal amino acid may be copper-free conditions. For instance, (b) may be performed in the absence of copper (Cu) when the first reporter comprises the strained alkyne. The method may further comprise reacting a second reporter different from the first reporter with the C-terminus of the peptide. The second reporter may comprise an alkyne. The second reporter may comprise a non-strained alkyne. The reaction between the second reporter and the C-terminus of the peptide may be performed in the presence of copper (e.g., when the alkyne is a non-strained alkyne). In some cases, the first reporter does not react with the second reporter. In some cases, the internal amino acid is coupled to an azide that does not react with an alkyne coupled to the C-terminus of the peptide.

In another aspect, provided herein is a method for processing or analyzing a peptide, comprising: (a) providing the peptide comprising an amino acid coupled to a label, wherein the label comprises (i) a first reactive group that is configured to couple to a second reactive group that is coupled to a reporter moiety configured to emit a signal or (ii) a protecting group configured to prevent coupling between the label and the second reactive group; (b) bringing the peptide in contact with a mixture comprising the second reactive group; (c) with the peptide immobilized to at least one support, detecting a signal from the peptide; and (d) using the signal or signal change to identify the amino acid or an additional amino acid of the peptide. The signal or signal change can be used to identify at least a portion of a sequence of said peptide. The signal or signal change can be used to identify the full sequence of the peptide. The use of the signal or signal change to identify the amino acid or the additional amino acid of the peptide may be performed subsequent to (a)-(c). The label may comprise a first reactive group that reacts with the second reactive group in (b).

The label may comprise the first reactive group. The reporter moiety may comprise the second reactive group. The second reactive group may react with the first reactive group. In (a), the label may comprise the first reactive group. In (b), the second reactive group may react with the first reactive group. In (a), the label may comprise the first reactive group, and, in (b), the second reactive group may react with the first reactive group.

The peptide may be provided as a plurality of peptides. The peptide may comprise at least one peptide of the plurality of peptides. The plurality of peptides may comprise a first peptide and a second peptide. The peptide may be the first peptide or the second peptide of the plurality of peptides. The first peptide may comprise a first amino acid coupled to a first label. The second peptide may comprise a second amino acid coupled to a second label. The first peptide may comprise a first amino acid coupled to a first label and a second amino acid coupled to a second label. The second peptide may comprise a first amino acid coupled to a first label and a second amino acid coupled to a second label. The first peptide may comprise a first amino acid coupled to a first label and the second peptide comprises a second amino acid coupled to a second label. The first label may be configured to react with the second reactive group. The second label may be configured to react with the second reactive group. The first label may be configured to react with the second reactive group and the second label may be configured to react with a different second reactive group than said second reactive group. The difference in the second reactive group configured to couple to each of the first label and the second label may allow selective coupling of a first label to a first amino acid and a second label to a second amino acid. Each second reactive group may be coupled to a reporter moiety independently configured to emit a signal. For example, the different second reactive group may be coupled to a second reporter moiety configured to emit a different signal than said reporter moiety.

The peptide may be immobilized to the at least one support in (a) or (b). The peptide may be immobilized to the at least one support in (a). The peptide may be immobilized to the at least one support in (b). The peptide may be immobilized to the at least one support in (a) and (b). The method may further comprise immobilizing the peptide prior to (c). The method may further comprise immobilizing the peptide subsequent to (b). The peptide may be immobilized to the at least one support prior to the detection of the signal from the peptide. The peptide may be immobilized to the at least one support subsequent to contacting it with the mixture comprising the second reactive group. The peptide may be immobilized to a first support. The peptide may be immobilized to a second support. The peptide may be immobilized to a first support and then immobilized to a second support. The peptide may be immobilized to a first support, removed from the first support, and then immobilized to a second support. The peptide may be immobilized to one or more different supports prior to (d).

The plurality of peptides may be immobilized to a plurality of supports. The plurality of peptides may comprise at least 1, 10, 100, 1000, 10,000, 100,000, 1,000,000 or more peptides immobilized to the same support. At least one support may be a bead, a polymer matrix, an array, or any combination thereof. The at least one support may be a bead, a polymer matrix, or an array. The at least one support may be a bead and an array. The at least one support can be a bead. The at least one support can be an array. The array can be a surface. The array can be a slide. The slide can be a microscopic slide. The at least one support can be a microscopic slide. The at least one support can be a polymer matrix.

The support may be a solid support or a semi-solid support. The solid support or semi-solid support may be a bead. The bead may be a gel bead. The bead may be a polymer bead. The support may be a resin. Non-limiting supports may comprise, for example, agarose, sepharose, polystyrene, polyethylene glycol (PEG), or any combination thereof. The support may be a polystyrene bead. The support may include functional groups, such as, for example, amines, sulfhydryls, acids, alcohols, bromides, maleamides, succinimidyl esters (NHS), sulfosuccinimidyl esters, disulfides, azides, alkynes, isothiocyanates (ITC), or combinations thereof. The support may be a PEGA resin. The support may be an amino PEGA resin. The support may comprise an amine group. The support may include protected functional groups, such as, for example, Boc, Fmoc, alkyl ester, Cbz, or combinations thereof. The bead may contain a metal core. The bead may be a polymer magnetic bead. The polymer magnetic bead may comprise a metal-oxide. The support may comprise at least one iron oxide core.

The peptide may comprise a plurality of amino acids. At least one amino acid of the plurality of amino acids may be selected from the group consisting of lysine, cysteine, glutamic acid, aspartic acid, tyrosine, arginine, histidine, threonine, serine, glutamine, asparagine and tryptophan. The plurality of amino acids may comprise one or more amino acids, the one or more amino acids selected from the group consisting of lysine, cysteine, glutamic acid, aspartic acid, tyrosine, arginine, histidine, threonine, serine, glutamine, asparagine and tryptophan. The peptide may comprise one amino acid selected from the group consisting of lysine, cysteine, glutamic acid, aspartic acid, tyrosine, arginine, histidine, threonine, serine, glutamine, asparagine and tryptophan. The plurality of amino acids may comprise a non-natural amino acid. The plurality of amino acids may comprise a D-amino acid.

At least one amino acid of the plurality of amino acids may be coupled to a label. The plurality of amino acids may comprise at least two or more amino acid types. The at least two amino acid or more types may comprise a first amino acid type and a second amino acid type. The first amino acid type may be coupled to a first label. The second amino acid type may be coupled to a second label. The first amino acid type may be coupled to a first label and the second amino acid type may be coupled to a second label. The first label and the second label may each be coupled to a different reporter moiety. The plurality of amino acids may comprise at least two, three, four, five, six, seven, eight, nine, ten, eleven, or more amino acid types. The plurality of amino acids may comprise between two and twenty amino acid types. The plurality of amino acids may comprise between 4 and 18 amino acid types. The plurality of amino acids may comprise between 6 and 16 amino acid types. The plurality of amino acids may comprise between 8 and 14 amino acid types. The plurality of amino acids may comprise between 9 and 11 amino acid types. Less than all of the amino acid types of the plurality of amino acids may labelled. Each amino acid type of the at least two amino acid types may be coupled to a different label. The peptide may comprise at least four amino acid types, wherein each amino acid type of said at least four amino acid types are coupled to a different label. Less than all of the plurality of amino acids may be labelled. Each of the plurality of amino acids may be labelled.

An amino acid may be selected from the group consisting of glycine, alanine, isoleucine, leucine, valine, phenylalanine, tryptophan, tyrosine, asparagine, cysteine, glutamine, methionine, serine, threonine, arginine, histidine, lysine, aspartic acid, glutamic acid, glutamic acid, and proline. An amino acid may be a derivative of any amino acid provided herein (e.g., pyrrolysine, selenocysteine, or N-formylmethionine). An amino acid may be polar, uncharged, charged (e.g., negative or positive charged), aliphatic, aromatic, neutral (e.g., zwitter-ion), hydrophobic, or any combination thereof. An amino acid may be a natural or an unnatural amino acid. An amino acid may be a D or a L amino acid. An amino acid may be an a or R amino acid.

The plurality of amino acids may comprise at least two amino acid types, and each amino acid type of the at least two amino acid types may be coupled to a different label. The peptide may comprise at least three amino acid types, wherein each amino acid type of said at least three amino acid types are coupled to a different label. The peptide may comprise at least four amino acid types, wherein each amino acid type of said at least four amino acid types are coupled to a different label. The peptide may comprise at least five or six amino acid types, wherein each amino acid type of said at least five or six amino acid types are coupled to a different label. The peptide may comprise at least eight amino acid types, wherein each amino acid type of said at least eight amino acid types are coupled to a different label. The peptide may comprise at least ten amino acid types, wherein each amino acid type of said at least ten amino acid types are coupled to a different label. Each label coupled to a different amino acid type may independently be coupled to a reporter moiety configured to emit a signal corresponding to each amino acid type. In some cases, the majority of the plurality of amino acids are labelled. In some cases, the majority of the plurality of amino acids are unlabeled.

The amino acid that can be coupled to a label may be an amino acid selected from the group consisting of lysine, cysteine, glutamic acid, aspartic acid, tyrosine, arginine, histidine, threonine, serine, proline, asparagine, glutamine, and tryptophan. The amino acid that is coupled to a label may comprise a post-translational modification. The post translational modification can be glycosylation, acetylation, alkylation, biotinylation, glutamylation, glycosylation, isoprenylation, phosphorylation, lipolation, phosphopantetheinylation, sulfation, selenation, amidation, ubiquitination, hydroxylation, nitration, nitrosylation, citrullination, cyclization (such as N-terminal glutamate or glutamine cyclization), and SUMOylation. In some cases, a label may have specificity for a particular amino acid with a particular post-translational modification.

The peptide may comprise a plurality of amino acids coupled to a plurality of labels. The plurality of amino acids may comprise a plurality of amino acids coupled to a plurality of labels. The plurality of amino acids coupled to a plurality of labels may comprise a first amino acid coupled to a first label and a second amino acid coupled to a second label. The plurality of amino acids may comprise a plurality of first amino acids coupled to a plurality of first labels. The plurality of amino acids may comprise a plurality of second amino acids coupled to a plurality of second labels. The plurality of amino acids may comprise (i) a plurality of first amino acids coupled to a plurality of first labels and (ii) a plurality of second amino acids coupled to a plurality of second labels. The plurality of amino acids may comprise (i) a plurality of first amino acids coupled to a plurality of first labels and (ii) a plurality of second amino acids coupled to a plurality of second labels. The first label, or the plurality thereof, may couple only to the first amino acid, or the plurality thereof. The second label, or the plurality thereof, may couple only to the second amino acid, or the plurality thereof. The first label, or the plurality thereof, may couple only to the first amino acid, or the plurality thereof, and the second label, or the plurality thereof, couples only to the second amino acid, or the plurality thereof. At least one label of the plurality of labels may be coupled to a specific amino acid type of the plurality of amino acids. For example, one label of the plurality of labels may be coupled to a lysine, a cysteine, a glutamic acid, an aspartic acid, a tyrosine, an arginine, a histidine, a threonine, a serine, a glutamine, an asparagine, or a tryptophan.

A label may comprise a first reactive group that is configured to couple to a second reactive group. The first reactive group may be selected from the group consisting of an azide, an alkyne, an alkene, an aldehyde, a ketone, a tetrazine, a thiol, a dithiol, a cyclooctene, and norbornene. The second reactive group may be selected from the group consisting of an azide, an alkyne, an alkene, an aldehyde, a ketone, a tetrazine, a thiol, a dithiol, a cyclooctene, and norbornene. The first reactive group may be selected from the group consisting of an azide, an alkene, an aldehyde, a ketone, and a tetrazine. The first reactive group may be a strained alkyne. The second reactive group may be selected from the group consisting of an azide, a thiol, a dithiol, a cyclooctene, an alkene, an aldehyde, a ketone, a tetrazine, a norbornene, and an alkyne.

The second reactive group may be a strained alkyne.

At least one label of the plurality of labels may be configured to react with a specific second reactive group coupled to a specific reporter moiety. The first reactive group may be selected from the group consisting of an alkyne, a thiol, a dithiol, and a cyclooctene, and the second reactive group may be selected from the group consisting of an alkyne, an azide, a thiol, a dithiol, a cyclooctene, an alkene, an aldehyde, a ketone, a tetrazine, and a norbornene. The first reactive group may be configured to react to a particular second reactive group. For example, the first reactive group may be an azide and the second reactive group may be an alkyne, the first reactive group may be an alkyne and the second reactive group may be an azide, the first reactive group may be an alkene and the second reactive group may be a thiol, the first reactive group may comprise an carbonyl (e.g., a ketone or an aldehyde) and the second reactive group may be an dithiol, the first reactive group may be a tetrazine and the second reactive group may be a cyclooctene (e.g., trans-cyclooctene).

The at least one label that couples to the amino acid, or plurality thereof, may be coupled to a reporter moiety. The reporter moiety may be configured to emit a signal. The reporter moiety may be configured to emit a signal upon excitation. The signal can be a detectable signal. For example, the signal can be an optical signal, such as a fluorescent or phosphorescent signal. The optical signal may be produced by a dye. The reporter moieties may also produce non-optically detectable signals. For example, a reporter moiety may produce an electrical signal, a radioactive signal or a chemical signal. The reporter moiety may be coupled to a spacer. The spacer may adjoin a reporter moiety and a second reactive group. A reporter may be configured to react with the label. The reporter may comprise a reporter moiety and a reactive group (e.g., a second reactive group). The reporter may comprise a reporter moiety, a reactive group (e.g., a second reactive group), and a spacer.

The method may comprise subjecting the peptide to conditions sufficient to remove at least one amino acid from the peptide immobilized to the at least one support. The at least one amino acid may be removed from an N-terminus of said peptide. The conditions sufficient to remove at least one amino acid from the peptide immobilized to the at least one support may be Edman degradation conditions. The conditions sufficient to remove at least one amino acid may comprise an Edman degradation agent or an organophosphate-containing agent. Subsequent to (e), the amino acid coupled to the label may become a terminal amino acid.

The method may further comprise (f) repeating steps (d) and (e) to detect at least one additional signal or signal change from said peptide immobilized to said at least one support and (ii) using the signal or signal change and the additional signal or signal change to identify at least a portion of a sequence of said peptide immobilized to said at least one support.

An amino acid or an additional amino acid of the peptide may be identified subsequent to (a)-(c). The at least one signal or signal change may be detected with an optical detector. The optical detector may comprise an imaging system. The optical detector may comprise single-molecule sensitivity. The at least one signal or signal change may comprise an optical signal. The at least one signal or signal change may be an optical signal. The at least one signal or signal change may comprise a plurality of signals. The at least one signal or signal change may comprise a plurality of signals of different frequencies or frequency ranges. The at least one signal or signal change may comprise a plurality of signals of different intensities. Two signals from among the plurality of signals may differ in at least one aspect. Two signals from among the plurality of signals may have different frequencies or frequency ranges. Two signals from among the plurality of signals may have different lifetimes. Two signals from among the plurality of signals may have different intensities.

The at least one signal or signal change may be generated by the reporter moiety. The reporter moiety may comprise a dye. The dye may generate the at least one signal or signal change. The dye can be selected from the group consisting of fluorescent dyes, phosphorescent dyes, chemiluminescent dyes, pigments, and photoswitchable reporters.

The protecting group may be configured to prevent coupling between the label and a second reactive group. The label containing the protecting group may act as a dummy label. The dummy label may block reactivity of a reactive group from reacting with an amino acid. The protecting group may comprise one or more groups. The one or more groups may be independently selected from the group consisting of azide, alkyl, alkylene, aryl, heteroaryl, heteroaryl-alkyl, and aryl-alkyl. The one or more groups may comprise an azide (e.g., N₃ or —CH₂N₃). The one or more groups may comprise an alkyl (e.g., methyl) or alkylene. The one or more groups may comprise an aryl (e.g., phenyl), heteroaryl, heteroaryl-alkyl, or aryl-alkyl (e.g., benzyl).

The method may be conducted at peptide concentrations of at most 0.001 nanomolar (nM), 0.01 nM, 0.1 nM, 1 nM, 10 nM, 100 nM, or more. The method may be conducted at peptide concentrations of at least 100 nM, 10 nM, 1 nM, 0.1 nM, 0.01 nM, 0.001 nM, or less. The method may be conducted at peptide concentrations from 0.001 nM to 100 nM, 0.01 nM to 100 nM, or 0.1 nM to 50 nM.

In another aspect, described herein is a method for coupling a peptide to a support (as described herein). The method may comprise reacting an agent coupled to the peptide to the support. The support may comprise a group (e.g., an alkyne, an azide, or any reactive group described herein) that is configured to react with the agent coupled to the peptide. The method may allow a first peptide to be coupled to a first support (e.g., through a first group) and a second peptide to be coupled to a second support (e.g., through a second group).

A method may comprise coupling a peptide to a bead, labeling the peptide, and coupling the peptide to a surface. In some cases, the peptide is released from the bead prior to its coupling to the surface. In some cases, the peptide is coupled to both the bead and surface. In some cases, the peptide is coupled to the bead by a cleavable linker. In such cases, the peptide may be released from the bead by cleavage of the cleavable linker prior to being coupled to the surface. Conversely, the peptide may be released from the bead after being coupled to a surface.

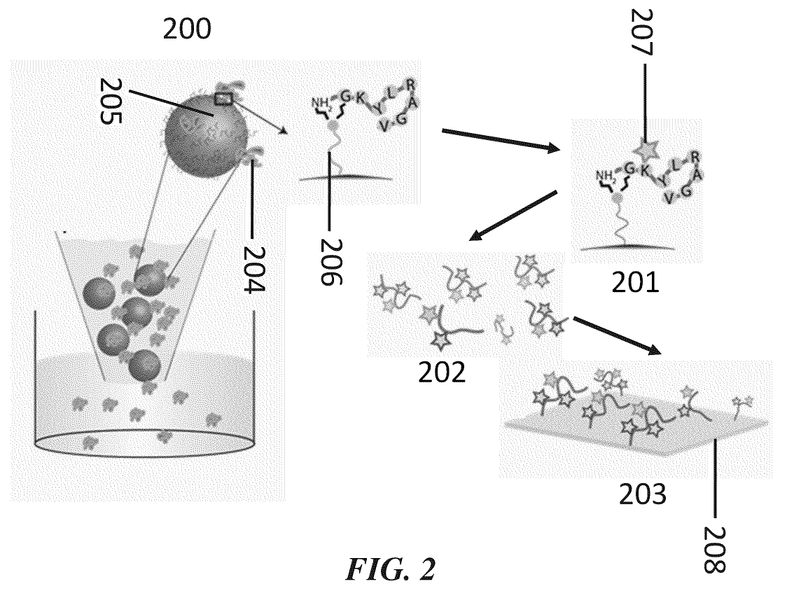

FIG. 2 outlines a method consistent with the present disclosure. The method may comprise capturing 200 a plurality of peptides 204 on a plurality of beads 205. The beads may comprise capture reagents 206 configured to couple to the plurality of peptides 204. The capture reagents may be N-terminal capture reagents, C-terminal capture reagents, amino acid type specific capture reagents, or may comprise a degree of binding non-specificity. The method may comprise labeling 201 the plurality of peptides. Such a step may comprise coupling an amino acid type specific label 207 to the plurality of peptides. Such a step may comprise coupling a plurality of labels to the plurality of peptides. Subsequent to labeling, the plurality of peptides may be released from the plurality of beads 202. The peptides may be coupled 203 to a surface 208 and subjected to analysis.

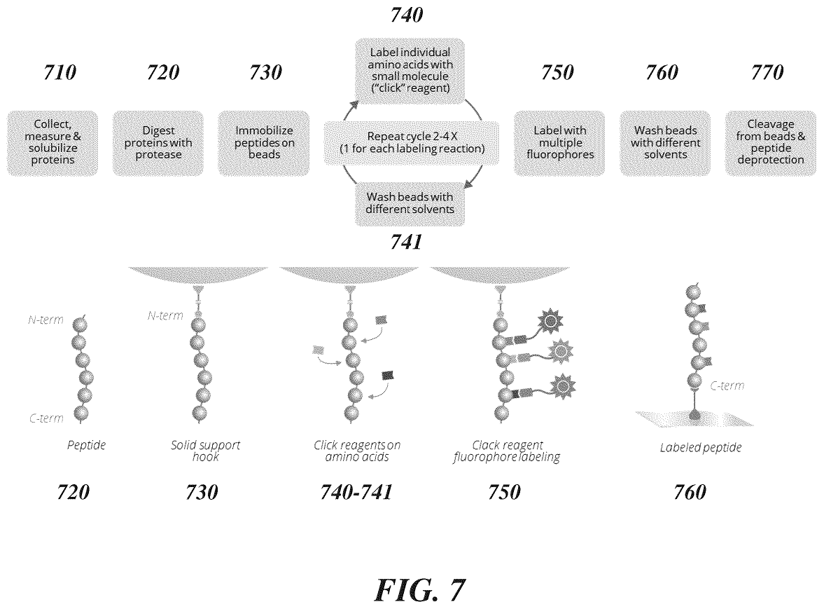

FIG. 7 illustrates method for labeling and immobilizing a peptide consistent with the present disclosure. The method may comprise collecting, optionally measuring, and optionally solubilizing (e.g., by addition of a chaotropic agent such as urea) a plurality of peptides 710. The plurality of peptides may be digested 720 to generate a plurality of peptide fragments. The plurality of peptide fragments may then be immobilized 730 on a bead (e.g., a plurality of beads), optionally washed or purified (e.g., to separate non-bead bound peptides from the bead), and coupled 740 to labels (e.g., “click” reagents). The purifying may comprise magnetic separation of the beads from solution. In some cases, the plurality of peptide fragments is coupled to a plurality of different labels (e.g., labels configured to couple to different types of amino acids). In such cases, two labels may be contacted to the plurality of peptide fragments at different times, interspersed by a wash step 741 to remove excess (e.g., unreacted) labeling reagents or side products from labeling steps. In some cases, no more than one label is contacted to the plurality of peptide fragments during each coupling step. In some cases, multiple labels are contacted to the plurality of peptide fragments during a single coupling step. The labels may be coupled 750 to reporter moieties, protecting groups, or any combination thereof. In some cases, multiple labels are coupled 750 to reporter moieties in a single step. In some cases, multiple labels are coupled 750 to reporter moieties over a plurality of steps (e.g., one reporter moiety and label are coupled in each step). The beads may be washed 760 to remove excess labeling reagents, and then coupled 770 to a substrate, such as a glass slide or a lantern, wherein the plurality of peptide fragments may be subjected to analysis, such as fluorosequencing. The peptides may optionally be released from the beads prior or subsequent to being coupled 770 to the substrate.

In another aspect described herein is a method for processing or analyzing a peptide, comprising: providing said peptide comprising an amino acid coupled to a first reactive group that is configured to couple to a second reactive group coupled to a support; bringing the peptide in contact with the second reactive group to permit the first reactive group to couple to the second reactive group, thereby immobilizing the peptide to said support; with said peptide immobilized to said support, detecting a signal from a label coupled to an amino acid of said peptide; and using said signal or change thereof to identify said amino acid.

The method can further comprise detecting a signal from a label coupled to an amino acid of the peptide immobilized to the support. The signal or change thereof can be used to identify the amino acid. The amino acid that can be coupled to the label that can be detected and the amino acid coupled to the first reactive group may be the same amino acid. The amino acid that is coupled to the label that is detected and the amino acid coupled to the first reactive group may be different.

The first reactive group may be coupled to the amino acid via a functional group. The functional group may be coupled to a reactive side chain of the amino acid. (a) may further comprise coupling the amino acid to a functional group coupled to the first reactive group. The functional group may only couple to the amino acid in (a). The amino acid in (a) may be the same as said amino acid coupled to said label in (c). The amino acid in (a) may be different from said amino acid coupled to said label in (c). (c) may further comprise coupling said label to said amino acid of said peptide prior to (b). (c) may further comprise coupling said label to said amino acid of said peptide subsequent to (b).

The amino acid coupled to a first reactive group may be a terminal amino acid. The amino acid coupled to a first reactive group may be an internal amino acid. The amino acid may be selected from the group consisting of lysine, cysteine, glutamic acid, aspartic acid, tyrosine, arginine, histidine, threonine, serine, proline, glutamine, and tryptophan. The amino acid may be selected from the group consisting of lysine, cysteine, glutamic acid, aspartic acid, tyrosine, arginine, threonine, serine, glutamine, and tryptophan.

The first reactive group may be selected from the group consisting of an azide, an alkyne, an alkene, an aldehyde, a ketone, a tetrazine, a thiol, a dithiol, an ester, a cyclooctene, and norbornene or activated ester (e.g., EDC-carbodiimide). The first reactive group may be an azide or an alkyne. The first reactive group may be an azide. The first reactive group may be an alkyne. The ester may be an activated ester. The activated ester may be EDC-carbodiimide.

The second reactive group may be selected from the group consisting of an alkyne, an azide, a thiol, a dithiol, a cyclooctene, an alkene, an aldehyde, a ketone, a tetrazine, and norbornene. The second reactive group may be an azide or an alkyne. The second reactive group may be an azide. The second reactive group may be an alkyne. The ester may be an activated ester. The activated ester may be EDC-carbodiimide. The first reactive group and said second reactive group may be coupled via a Click reaction. The second reactive group may be coupled to said support through a spacer (as described herein) or a linker. The linker may be a bond or an optionally substituted alkylene or an optionally substituted heteroalkylene.

The at least one support may be a bead, a polymer matrix, an array, or any combination thereof. The at least one support may be a bead, a polymer matrix, or an array. The at least one support may be a bead and an array. The at least one support can be a bead. The at least one support can be an array. The array can be a surface. The array can be a slide. The slide can be a microscopic slide. The at least one support can be a microscopic slide. The at least one support can be a polymer matrix.

The label may be coupled to a reporter moiety. The reporter moiety may be configured to emit a signal. The reporter moiety may be configured to emit a signal upon excitation. The reporter moiety may be configured to absorb light of a specific wavelength. The signal can be a detectable signal. For example, the signal can be an optical signal, such as a fluorescent or phosphorescent signal. The optical signal may be produced by a dye. The reporter moiety may also produce non-optically detectable signals. For example, a reporter moiety may produce an electrical signal, a radioactive signal, or a chemical signal. The signal may be from a nucleic acid. The nucleic acid may be a barcode. The barcode may be a DNA barcode or an RNA barcode. The reporter moiety may be coupled to a spacer.

The reporter moiety may be coupled to a third reactive group or an antibody. The reporter may comprise a reporter moiety and a reactive group (e.g., a third reactive group or an antibody). The reporter may comprise a reporter moiety, a reactive group (e.g., a third reactive group or an antibody), and a spacer. The third reactive group or the antibody may be configured to couple to the amino acid that is coupled to the label. The amino acid may be a terminal amino acid or an internal amino acid. The reporter moiety may be coupled to a third reactive group or an antibody by a linker. The signal can be detected from a label coupled to an amino acid, wherein the label is coupled to an antibody.

The method may further comprise subjecting said peptide to conditions sufficient to remove an amino acid from a terminal end of said peptide. The removal of the amino acid may be performed subsequent to (c). The at least one amino acid may be removed from an N-terminus of said peptide. The conditions sufficient to remove at least one amino acid from the peptide immobilized to the at least one support may be Edman degradation conditions. The conditions sufficient to remove at least one amino acid may comprise an Edman degradation agent or an organophosphate-containing agent. Subsequent to (b), the amino acid coupled to the label may become a terminal amino acid.

The method may further comprise repeating (c) and (d) one or more times to identify a signal pattern of a plurality of amino acids of said peptide, which said signal pattern of said plurality of amino acids comprises said signal or change thereof in (d). The method may further comprise using said signal pattern of said plurality of amino acids to obtain a sequence of said peptide, or a portion of the sequence of said peptide.

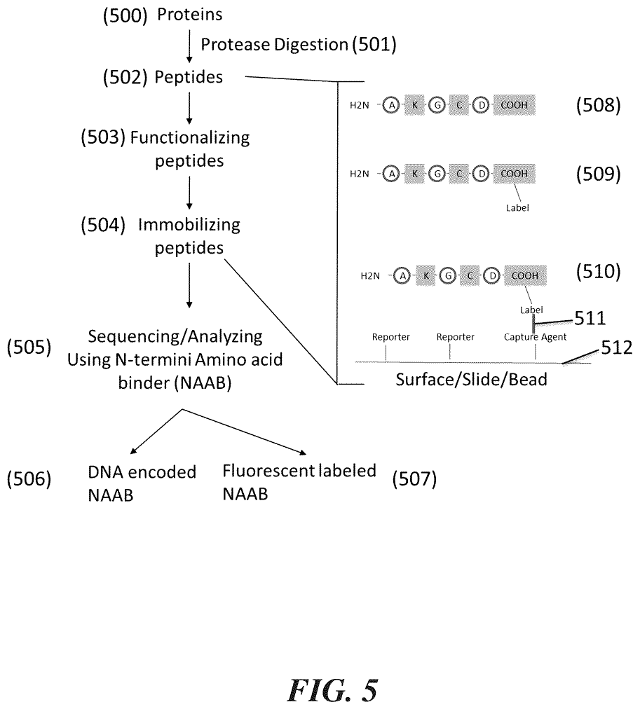

Alternatively, the method may be performed as shown in FIG. 5. The method may comprise a first peptide 500 that is digested 501 to at least one second peptide 502. A C-terminal of the at least one second peptide can be functionalized 503 with a label 509. The at least one second peptide may be immobilized 504 to a support 512 (such as a surface, a slide, or a bead), for example by coupling the label 509 to a capture agent 511 that is coupled to the support 512. The N-terminal amino acid of the at least one second peptide may then be coupled to an N-terminal binding agent 505, which may be coupled to a reporter moiety such as a barcoded nucleic acid 506 for detection by sequencing or a fluorophore 507 for optical detection. The N-terminal binding agent may comprise a specificity for an N-terminal amino acid type, such as lysine, or a group of amino acids, such as amino acids with side chains comprising aromatic groups. The barcode or fluorophore may identify the N-terminal amino acid type of the at least one second peptide to which the N-terminal binding agent is coupled. Optionally, the N-terminal amino acid of the at least one second peptide may be removed, exposing a new N-terminal amino acid which may be contacted by a further N-terminal binding agent. Such N-terminal amino acid identification and removal may be repeated over a number of cycles to identify a sequence of the at least one second peptide. In some cases, the N-terminal amino acid is functionalized prior to its coupling to an N-terminal amino acid binding agent. For example, an N-terminal amino acid may be coupled to a label comprising a second reactive group, followed by coupling of an N-terminal binding agent recognition group to the second reactive group of the label, followed by N-terminal binding agent coupling to the N-terminal amino acid comprising the N-terminal binding agent recognition group. Such an N-terminal binding agent recognition group may comprise an affinity for the N-terminal binding agent.

### 4. Kits

In another aspect, provided herein is a kit for analyzing a peptide. In some aspects, the kit is for assaying a sequence of a peptide. The kit may be used in a device capable of sequencing nucleic acids. The kit may be packaged in a cartridge configured to be used in a device capable of sequencing nucleic acids.

In another aspect, provided herein is a kit for assaying a sequence of a peptide in a sample, comprising a label comprising a first reactive group and (i) a second reactive group or (ii) a protecting group, wherein said first reactive group is configured to couple to an amino acid of an amino acid type, wherein said second reactive group is configured to couple to a reporter moiety, comprising a reporter moiety configured to emit a signal, and wherein said protecting group is configured to prevent coupling between said label and said reporter moiety; and instructions for using said label to process said peptide to provide said peptide comprising said amino acid coupled to said label. The first reactive group may be configured to couple to an amino acid of an amino acid type. The second reactive group may be configured to couple to the reporter. The reporter may be coupled to a third reactive group configured to the second reactive group. The reporter may comprise a spacer. The reporter may be configured to emit a signal upon excitation. The reporter may comprise a fluorescent dye. The protecting group may be configured to prevent coupling between the label and the reporter. The protecting group may not emit an optically detectable signal.

The kit may comprise a protein capture agent. The protein capture agent may be configured to couple to an N-terminus of the peptide. The protein capture agent may comprise a solid support coupled to a cleavable linker. The protein capture agent may be coupled to a solid support by a cleavable linker. The solid support may comprise a bead, an array, a slide, a polymer matrix, or any combination thereof. The cleavable linker may be cleavable by an enzyme. The cleavable linker may be a chemically cleavable linker. The cleavable linker may be a photocleavable linker. The cleavable linker may be capable of being cleaved by a change in pH. The cleavable linker may comprise an aldehyde. The aldehyde may be pyridinecarbaldehyde (PCA) or a derivative of PCA.

A capture reagent may react with at least one peptide or protein. A capture reagent may react with the N-terminus of at least one peptide or protein. A capture reagent may react with the C-terminus of at least one peptide or protein. A capture reagent may react with one peptide or protein. A capture reagent may react with the N-terminus of one peptide or protein. A capture reagent may react with the C-terminus of one peptide or protein. Each peptide or protein of a cell may be captured by a plurality of capture reagents. The support may further comprise a capture reagent that can capture a molecule that is not a peptide or protein. The support may further comprise a capture reagent that can capture a nucleic acid molecule. The support may further comprise a capture reagent that can capture a ribonucleic acid molecule.

The reporter (or reporter moiety) may be configured to emit a signal. The reporter (or reporter moiety) may comprise a dye. The dye may be selected from the group consisting of fluorescent dyes, phosphorescent dyes, chemiluminescent dyes, pigments, and photoswitchable reporters. The reporter (or reporter moiety) may comprise a fluorescent dye. The reporter may be configured to emit the signal upon excitation. The reporter may be a fluorescent protein molecule.

The kit may comprise a surface attachment agent. The surface attachment agent may comprise an alkyne or an azide. The surface attachment agent may be configured to couple to a C-terminus of a peptide. A kit may comprise a support to which the surface attachment agent attaches. In some cases, the support is a slide. The slide may be a glass slide. The slide may be a microscopic slide.

The kit may comprise additional agents useful for carrying out a reaction, handling a peptide or any of the reagents described herein, or performing analysis. A kit may comprise one or more species from the group consisting of proteases, digestion reagents, solid support beads, or any combination thereof. A kit may also comprise small molecules, buffers, and solvents useful for carrying out a reaction. A kit may come pre-packaged in a container set. The prepackaging may be a cassette configured to be used in any sequencing platform.

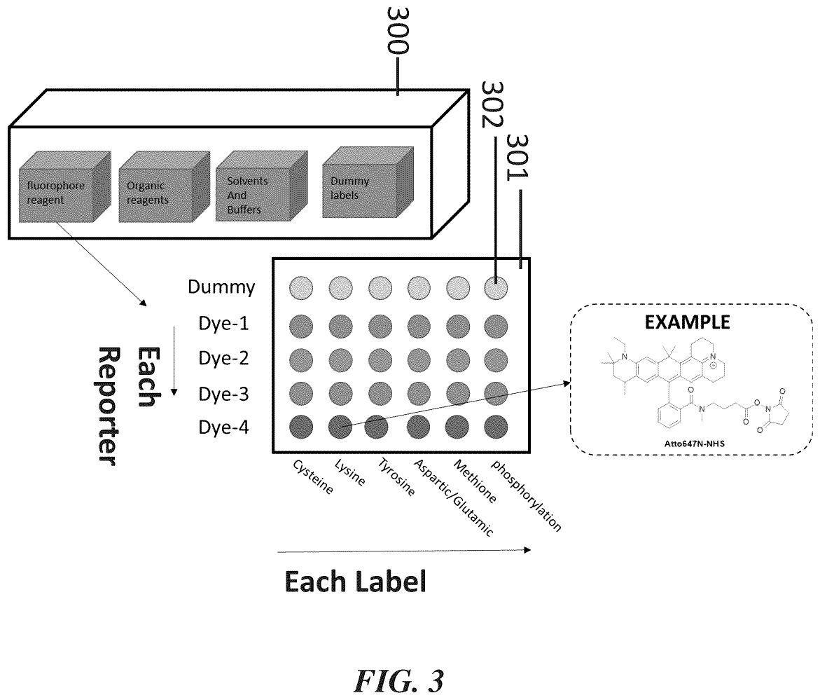

FIG. 3 illustrates a kit consistent with the present disclosure. The kit may comprise a substrate 301 comprising a plurality of volumes 302 (e.g., a well plate comprising a plurality of wells). Each volume of the substrate may be specified for a reagent. For example, the substrate may comprise an array of volumes wherein each row may correspond to a different reporter moiety and each column may correspond to a different target amino acid type. The kit may comprise a series of reagents 300 for handling, labeling, and detecting peptides, including prefabricated labels with reporter moieties. Accordingly, every combination of amino acid label-type and reporter moiety-type needs to be included in a separate container or volume of the kit. For example, if the kit contains 6 types of amino acid labels combinatorially combined with 6 types of reporter moieties, the kit needs to include at least 36 separate containers or volumes (at least one for each label-reporter moiety combination).

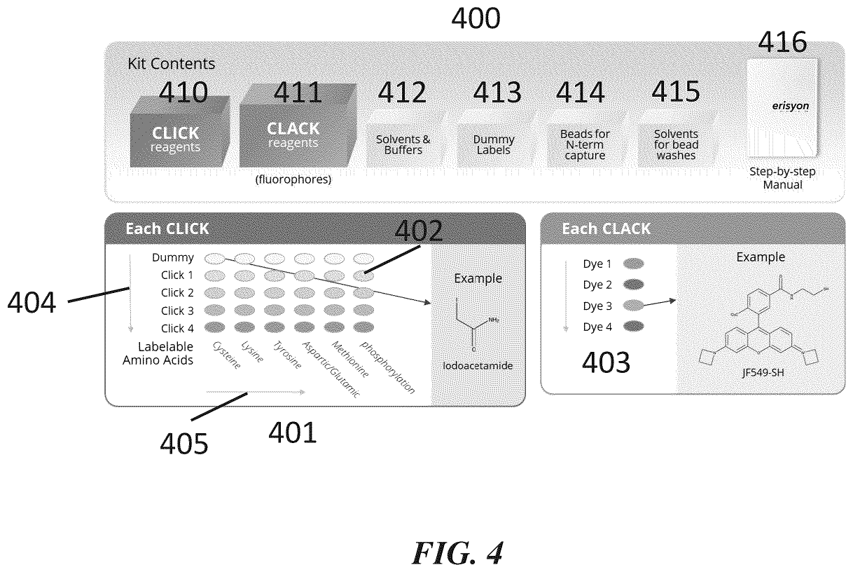

A kit of the present disclosure may be configured as shown in FIG. 4. Reagents 400 may be provided as a set, and may include “click” reagents 410 (e.g., labels comprising first reactive groups for coupling to targets and second reactive groups for coupling to “clack” reagents), “clack” reagents 411 (e.g., reporter moieties or protecting groups configured to couple to second reactive groups of “click” reagents), solvents and buffers 412, dummy labels 413 (e.g., labels comprising first reactive groups for coupling to targets but lacking second reactive groups for coupling to “clack” reagents), beads 414 (e.g., beads comprising capture agents for N-terminal or C-terminal peptide capture), and reagents for wash steps 415. The kit may comprise a manual 416 outlining methods for use of the kit. The kit may comprise a well plate 401 comprising wells 402 in which the “click” and “dummy” labels may be coupled to peptides, and “click” labels may be coupled to “clack” reagents. In some configurations, the wells of the well plate may be used to construct detectable labels (e.g., couple “click” labels to “clack” reagents), such that each row and column of the plate specifies a “click” and “clack” pair. The well plate may comprise rows corresponding to label type 404 and columns corresponding to target amino acid type 405. The clack reagents may comprise a range of reporter moieties 403, such as a plurality of differently colored dyes. This kit design may enable generation of a large library of label-reporter moiety combinations from a relatively smaller set of reagents. For example, a kit consistent with FIG. 4 may comprise 6 types of reporter moieties and 6 types of amino acid specific labels, thereby enabling the generation of 36 separate label-reporter moiety conjugates.

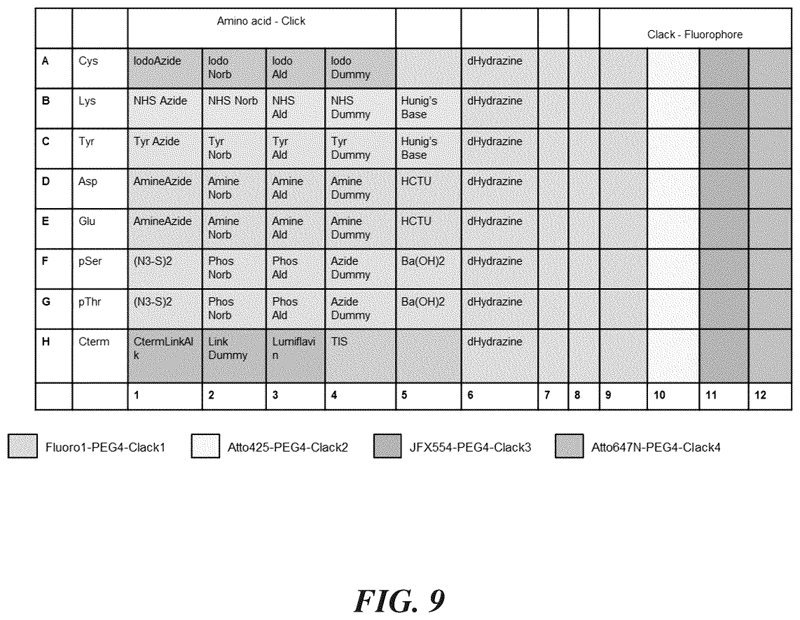

FIG. 9 illustrates a kit design consistent with the present disclosure. The kit may comprise a substrate 900 comprising a plurality of volumes. The volumes may comprise labels configured to couple to specific amino acid types, as well as a range of “clack” reagents for coupling to the labels. Different rows of the substrate may correspond to different amino acid types (in the kit shown, rows A-H correspond to cysteine, lysine, tyrosine, aspartic acid, glutamic acid, phosphoserine, phosphothreonine, and C-terminal amino acids, respectively). In this kit, the cysteine “click” labels include and iodoacetamide label with an azide second reactive group, an iodoacetamide label norbornenone second reactive group, an iodoacetamide label with an aldehyde second reactive group, and an iodoacetamide “dummy” label lacking a second reactive group. The lysine “click” labels include an NHS ester label with an azide second reactive group, an NHS ester label with a norbornenone second reactive group, an NHS ester label with an aldehyde second reactive group, and an NHS ester “dummy” label lacking a second reactive group. The tyrosine “click” labels include an aryl diazonium label with an azide second reactive group, an aryl diazonium label with a norbornenone second reactive group, an aryl diazonium label with an aldehyde second reactive group, and an aryl diazonium “dummy” label. The aspartic acid and glutamic acid “click” labels include an amine label with an azide second reactive group, an amine label with a norbornenone second reactive group, an amine label with an aldehyde second reactive group, and an amine “dummy” label. The phosphoserine and phosphothreonine labels include reagents, such as Barium hydroxide, TCEP and labels, such as reagents comprising reactive thiols (e.g., cystamine). The kit includes reagents for coupling the “click” labels to peptides, such as Hunig's base, 2-(6-Chloro-1-H-benzotriazole-1-yl)-1,1,3,3-tetramethylaminium hexafluorophosphate (HCTU), TCEP and Ba(OH)₂. The kit also contains 4 different colors of “clack” detectable moieties to couple to the various “click” labels, including a range of fluorophore-PEG4, Atto425-PEG4, JFX554-PEG4, and Atto647N-PEG4 “clack” reagents, enabling any combination of the 4 colors with any “click” reagent. Other fluorophores such as Atto495, Janelia fluor 525 and Janelia Fluor 579 could also be used for making of these “clack” reagents as reporter molecules.

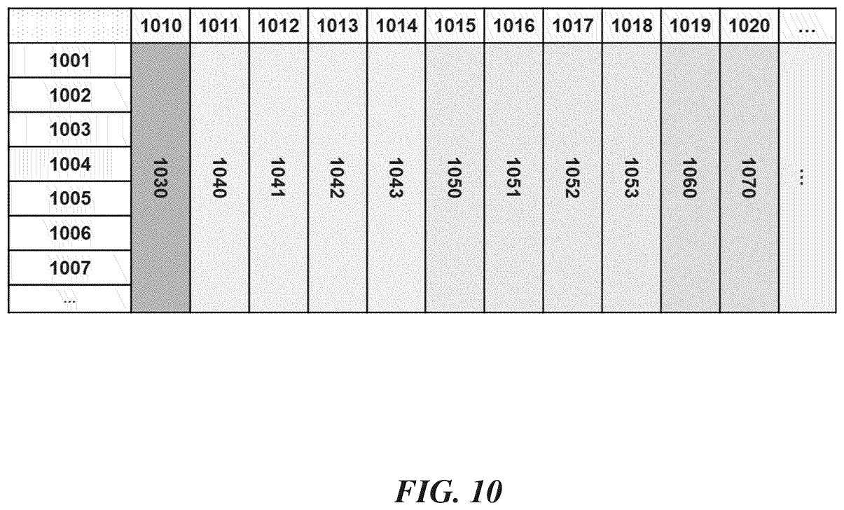

FIG. 10 illustrates a further kit design consistent with the present disclosure. The kit may comprise a substrate with a plurality of rows 1001-1007 and columns 1010-1020 comprising a plurality of sets of reagents 1030, 1040-1043, 1050-1053, 1060, 1070 for processing peptides. Each set of reagents may comprise a different reagent in each row. The substrate may include reagents for peptide immobilization 1030, labels for coupling to peptides 1040-1043 (e.g., “click” labels), reagents for coupling to labels 1050-1053 (e.g., “clack” reporter moieties or protecting groups), wash reagents 1060, and reagents for coupling and decoupling peptides from solid supports 1070. The substrate may comprise at least one volume (e.g., at least one well) for each row and column pair.

A method may comprise tailoring a kit or a composition for a particular method, sample type, or desired performance level (e.g., sensitivity). A Combination of reagents (e.g., labels, reporter moieties, solvents, buffers, proteases) may be selected to optimize for a particular experiment or inquiry. A kit may enable such optimization by providing a plurality of reagents from which a subset may be selected.

Optimal label combinations for a single molecule sequencing workflow can vary depending on the type of organism being studied and the concentrations of the molecules to be identified. As amino acid compositions vary between organisms, a label selection (e.g., a cysteine label, a lysine label, and a tryptophan label) amenable for peptide discrimination in a first organism may not be optimal for peptide discrimination in a second organism. Accordingly, a method of the present disclosure may comprise selecting a set of labels based on the type of sample (e.g., human serum) or organism (e.g., Pichia pastoris) queried.

Reagents may be optimized for sample compatibility. A method may comprise selecting a reagent with a low cross-reactivity within a sample. For example, some fluorophores are known to intercalate into nucleic acids (for example, flavonoid-based dyes can rapidly intercalate into B-DNA present in many samples) and may therefore be excluded from methods which utilize nucleic acid-containing samples. A method may comprise selecting a reagent (e.g., a reporter moiety) which comprises a half-life of at least 1 hour, at least 2 hours, at least 4 hours, at least 6 hours, at least 8 hours, at least 12 hours, at least 16 hours, at least 24 hours, at least 36 hours, at least 48 hours, at least 60 hours, at least 72 hours, at least 90 hours, or at least 120 hours at 25° C. in a sample from which an analyte was derived. A method may comprise selecting a reagent (e.g., a reporter moiety) which comprises a half-life of at least 1 hour, at least 2 hours, at least 4 hours, at least 6 hours, at least 8 hours, at least 12 hours, at least 16 hours, at least 24 hours, at least 36 hours, at least 48 hours, at least 60 hours, at least 72 hours, at least 90 hours, or at least 120 hours at 25° C. in a sample in which an analyte is interrogated (e.g., a buffer for fluorosequencing). A method may comprise selecting a reagent which comprises tolerance for a pH, a temperature, a salinity, or a reactive species of a sample.

A combination of fluorophores provided in a kit or used in a method of the present disclosure can be optimized for a range of methods. The types of fluorophores selected for a method or provided in a kit may be selected to: (i) diminish quenching and (ii) diminish spectral overlap (excitation and/or emission spectral overlap). For example, the optimal types of fluorophores for a four-color fluorosequencing method may be different than the optimal types of fluorophores for a two-color cell imaging stain. While the fluorosequencing method may generate relatively short inter-fluorophore distances from multi-fluorophore coupling to single peptides, and therefore require low-quenching activity fluorophore pairs, the stain method may comprise a higher background signal from a milieu of optically active cellular materials, and therefore favor higher quantum efficiency at the expense of higher quenching.

A set of reagents or a kit may also be optimized so that multiple reactions may be performed in a single step. A kit of the present disclosure may comprise a plurality of “click” labels and corresponding “clack” reporter moieties or protecting groups which reactive group pairs amenable for single step coupling reactions. For example, a kit may be configured for a method in which cysteine residues present in a sample are coupled to a first label, lysine residues present in the sample are coupled to a second label, tryptophan residues present in the sample are coupled to a third label, and then all three labels are coupled to distinct “clack” reporter moieties (e.g., cystine labels are coupled to blue reporter moieties, lysine labels are coupled to red reporter moieties, and tryptophan labels are coupled to green reporter moieties) in a single step. A method may comprise coupling at least two “clack” reporter moieties or protecting groups to at least two “click” labels in a single step. A method may comprise coupling at least three “clack” reporter moieties or protecting groups to at least three “click” labels in a single step. A method may comprise coupling at least four “clack” reporter moieties or protecting groups to at least four “click” labels in a single step. A method may comprise coupling at least five “clack” reporter moieties or protecting groups to at least five “click” labels in a single step. The single step may be a step in which all “clack” reporter moieties are added simultaneously, react with labels simultaneously, or any combination thereof.

A method may comprise computationally designing a kit or set of reagents consistent with the present disclosure. A user may input the type of method, the required sensitivity, the sample conditions (e.g., in vivo, ex vivo, or in vitro; pH, temperature, target concentration, etc.) into a program configured to optimize the set of reagents. The program may search a database of known proteomes, reactive species (e.g., glutathione concentration), and cytosolic conditions for a plurality of samples and organisms to generate an optimal reagent set. For example, the program may select a protease and a set of amino acid specific labels based on the amino acid abundances of a target organism.

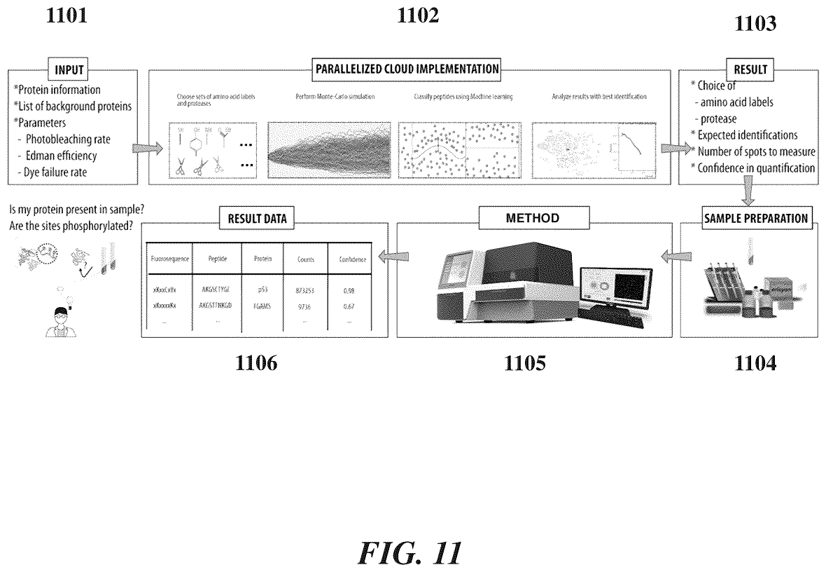

FIG. 11 illustrates a method for designing a kit consistent with the present disclosure. The method may be performed on a processing device, such as a smartphone. The user may input 1101 information regarding their intended method, such as protein information (e.g., the types of proteins present in the samples to be queried), a list of background species (e.g., proteins) not targeted by their method, sample and experimental parameters, photobleaching rate, chemical or process efficiency (e.g., the expected efficiency of an Edman reaction to be performed in a fluorosequencing method), and dye failure rate. The processing device may then input the user provided information into a computational model 1102 to output 1103 an optimal choice of reagents (e.g., label and reporter moiety combinations) for the given method which the user intends to perform based on the input information. The output 1103 may further include (e.g., through a parallelized cloud implementation of a single molecule sequencing workflow) experimental protocols, such as the number of expected protein identifications from a protein fluorosequencing experiment, the number of spots to measure, the number of replicates to include, and the expected confidence in quantitation. The user may then generate 1104 the recommended set of reagents (e.g., a kit) comprising the output reagents. In some cases, the user may generate the recommended set of reagents by coupling “click” labels with “clack” reporter moieties and protecting groups. In some cases, the user may order the labels and other reagents (e.g., proteases) for use. The user may perform their method with the reagents 1105, and optionally may work up or analyze 1106 their data based on their intended experiment.

### 4.1 Amino Acid Specific Agents

An amino acid specific agent described herein may be an amino acid specific bifunctional linker, also referred to as a “label.” Labels may contain two or more reactive groups (e.g., portions or regions of the label). The first reactive group may be configured to couple to a specific amino acid type or a set of specific amino acid types. In some instances, the first reactive group is capable of differentiating between two similar amino acids (e.g., tyrosine and serine), and may be configured to couple to one type of amino acid or a specific number of types of amino acids. In other examples, the first reactive group is configured to couple to a plurality of amino acid types (e.g., a first reactive group may be configured to couple to carboxylate containing amino acids, including glutamate, aspartate, and C-terminal amino acids). In specific cases, a first functional group be capable of coupling to any amino acid.

A first reactive group may couple to the side chain of an amino acid. Alternately, a label may couple to the backbone of an amino acid. In some cases, labels are selective for a position within a peptide. (e.g., terminal residues, internal residues, residues adjacent to a specific amino acid). Some labels couple (e.g., selectively couple) to post-translationally modified sites on an amino acid. Some non-limiting examples of post-translational modifications (to an amino acid) that can be coupled to a label include glycosylation, acetylation, alkylation, biotinylation, glutamylation, glycylation, isoprenylation, phosphorylation, lipolation, phosphopantetheinylation, sulfation, selenation, amidation, ubiquitination, hydroxylation, nitrosylation, succinylation, carboxylation, oxidation, glutathionylation, halogenation, N-terminal amino acid cyclization, post-translationally derived cofactor formation (e.g., the green-fluorescent protein chromophore and tyrosine-cysteine crosslinking) and SUMOylation.

In some cases, a first reactive group comprises a functional group that couples to a specific amino acid side chain. Some non-limiting examples of functional groups include iodoacetamide, succinimidyl esters, N-hydroxysuccinimide esters, alkyl amines, diazo compounds, thiols, methacrylic acid, and propargyl amine.

In some examples, the second reactive group is unreactive or inert. The unreactive second reactive group may not react with any other entities within the system. In some examples, the unreactive second reactive group serves as a protecting group. The protecting group may, for example, prevent coupling to a reporter (or reporter moiety). The protecting group may not emit a detectable signal. In some instances, a label with an inert or unreactive second reactive group is referred to as a “dummy label.” The present disclosure further provides that the dummy label may be converted to a reactive label. Converting a dummy label to a reactive label may be executed by an enzymatic process, an oxidative or reductive process, a photocatalyzed process, a metal catalyzed process, an acidic or basic process, or a radical process.

The second reactive group may be reactive. The first reactive group may be configured to couple to a reporter. The first reactive group may be capable of coupling to a second reactive group. In some examples, the second reactive group is disposed on a reporter, thereby coupling a label to a reporter. Such a label may be bifunctional, having an amino acid functionality and a reporter-coupling functionality. Labels provided herein may have more than one reactive domain or reactive group, thus enabling coupling to more than one reporter. A label with multiple reactive domains or reactive groups may be a multifunctional label.

By way of non-limiting example, select second reactive groups for use in coupling to a reporter moiety (or plurality of reporter moiety) may include an azide, an alkene, an aldehyde, a ketone, a tetrazine, an alkyne, a strained alkyne (e.g., a cycloalkyne, cyclooctyne, DBCO), a thiol, a dithiol, a trans-cyclooctene, a bicycloalkene, an iodobenzene, a cyanothiazole, acene, a dithiolane, a bromane, an aminothiol, a pyrroledione, or any combination thereof. Non-limiting examples of second reactive group-reporter moiety reactive group pairs include an azide and an alkyne, an azide and a dibenzocyclooctyne (DBCO), an alkene and a thiol, an aldehyde and a dithiol, a ketone and a dithiol, a tetrazine and a trans-cyclooctene, a tetrazine and a nobornen-NHS-ester, a bicycloalkene and a tetrazine, an aldehyde and a dithiolane, an iodobenzene and a bromane, a cyanothiazole and an aminothiol, and an acene and a pyrroledione. A reactive label may couple selectively to a specific reporter. Examples of reactive groups that may be configured to selectively couple are shown in TABLE 3.

TABLE 3
Examples of Reactive Groups with Selective Coupling Activity

| Scond Reactive | | Reporter Moiety | |
|---|---|---|---|
| Group | Structure | Reactive Group | Structure |
| Azo | 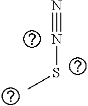 | Alkyne | 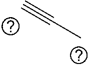 |
| Azide | 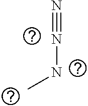 | DBCO (″strained alkyne″) | 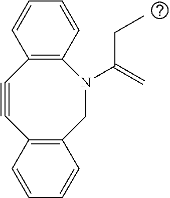 [SMILES: C=C(CC)N1Cc2ccccc2C#Cc2ccccc21] |
| Alkene | 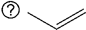 | Thiol | 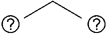 |
| Aldehyde/Ketone | 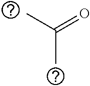 | Dithiol | 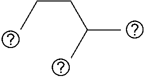 |
| Tetrazine | 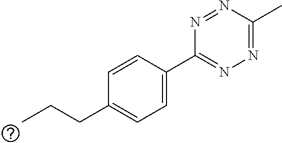 [SMILES: CCCc1ccc(-c2nnc(C)nn2)cc1] | Trans-cyclooctene | 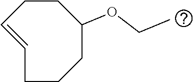 |
| Tetrazine | 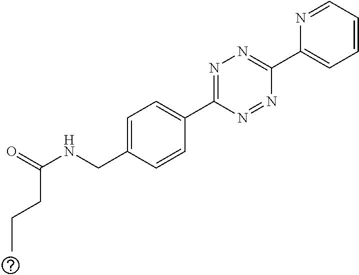 [SMILES: CCCC(=O)NCc1ccc(-c2nnc(-c3ccccn3)nn2)cc1] | Norbornene-NHS-Ester | 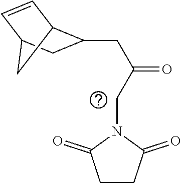 [SMILES: O=C(CC1CC2C=CC1C2)CN1C(=O)CCC1=O] |
| Bicycloalkene | 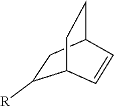 | Tetrazine | 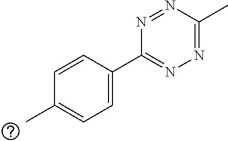 |
| Aldehyde | 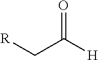 | Dithiolane | 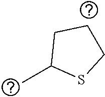 |
| lodobenzene | 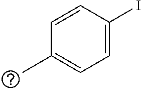 | Bromane | 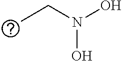 |
| Cyanothiazole | 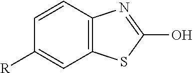 | Aminothiol | 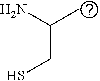 |
| Acene | 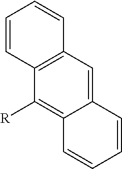 | Pyrroledione | 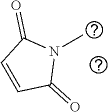 |

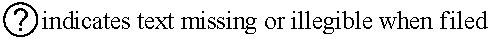

In some instances, a reactive label is non-selective and can couple to a plurality of reporters. In other instances, the second reactive group may be directly detectable (e.g., fluorescent) without coupling to a reporter. A label may contain other identifiable or detectable inclusions such as a barcode or an isotope. In some examples, the barcode is a molecular barcode containing an identifiable sequence of repeating units (e.g., nucleic acids). A second reactive group may be disposed on a non-reporter, which does not emit a detectable signal. A second reactive group may be disposed on a molecular construct that functions as a protecting group, which is substantially unreactive or inert.

In some cases, the kits may comprise a buffer. A buffer may refer to a species that can diminish the degree of pH change in a composition relative to the pH change that would occur if the buffer were not present. Non-limiting examples of buffers include In some cases, a system of the present invention may comprise a buffer selected from the group consisting of sodium phosphate, HEPES, MES, and citrate. In some cases, a buffer may be in the form of a solid. In some cases, a buffer may be dissolved in a liquid. In some cases, a buffer may be adsorbed onto solid.

A solution of the present invention may comprise a buffer. The buffer may be present at 1 nM or higher concentrations. In some cases, the buffer may be present at 10 nM or higher concentrations. In some cases, the buffer may be present at 100 nM or higher concentrations. In some cases, the buffer is present at 1 μM or higher concentrations. The buffer may be present at 10 μM or higher concentrations. In some cases, the buffer may be present at 100 μM or higher concentrations. In some cases, the buffer may be present at 1 mM or higher concentrations. In some cases, the buffer may be present at 10 mM or higher concentrations. In some cases, the buffer may be present at 100 mM or higher concentrations. In some cases, the buffer may be present at 200 mM or higher concentrations. In some cases, the buffer may be present at 500 mM or higher concentrations.

In some cases, the solution comprising a buffer may have a pH of between 2 and 12. In some cases, the solution comprising a buffer may have a pH of between 2 and 6. In some cases, the solution comprising a buffer may have a pH of between 3 and 7. In some cases, the solution comprising a buffer may have a pH of between 4 and 8. In some cases, the solution comprising a buffer may have a pH of between 5 and 9. In some cases, the solution comprising a buffer may have a pH of between 6 and 10. In some cases, the solution comprising a buffer may have a pH of between 7 and 11. In some cases, the solution comprising a buffer may have a pH of between 8 and 12. In some cases, the solution comprising a buffer may have a pH of around 7.

The systems of the present disclosure may comprise sodium phosphate. In some cases, a solution may comprise the sodium phosphate. In In some cases, the sodium phosphate may be monosodium phosphate. In some cases, the sodium phosphate may be disodium phosphate. In some cases, the sodium phosphate may be trisodium phosphate.

Reporters

Reporters of the present disclosure may be constructs that can be detected (e.g., optically detected, mass detected, radiometrically detected, chemically detected by reaction with another entity) and/or identified (e.g., by color, by mass, by decoding or sequencing). Reporters for use in the present disclosure may contain more than one domain. For example, a reporter may contain a reactive domain that can couple to the reactive domain of a complementary label. A reporter may contain a second reactive group that can couple to a first reactive group of a complementary label. In some instances, the second reactive group couples only with a specific label. In other examples, the second reactive group can couple to any label. Reporters may couple to more than one label. A reporter may contain a second reactive group that can couple directly to an amino acid (specifically or non-specifically). In this instance, the reporter can couple to the side chain or a post-translationally modified side chain of an amino acid. By way of non-limiting example, some second reactive groups for use in a reporter may be selected from the group consisting of an azide, an alkene, an aldehyde, a ketone, a tetrazine, an alkyne, a strained alkyne (e.g., a cycloalkyne, cyclooctyne, DBCO), a thiol, a dithiol, or a trans-cyclooctene. An advantageous aspect of the present disclosure is the pairing of labels and reporters wherein the functional groups are known and the coupling conditions are optimized to selectively couple a known reporter to a known label. An amino acid may be identified by the detectable or identifiable moiety within the reporter.

In addition to a reactive, coupling domain, reporters of the present invention may also contain a reporter moiety. A reporter moiety may contain an identifiable domain. In some examples, an identifiable domain is associated with a discrete address (e.g., a barcode or a frequency). In some examples, such a reporter moiety is configured to emit a signal upon excitation. In some examples, such a signal is an optical signal. The reporter may contain a non-detectable domain, wherein the reporter does not emit an optically detectable signal. A reporter may also contain an inert domain. An inert domain may be cleavable. An inert domain may function as a protecting group to prevent reactivity.

A reporter may emit a signal upon excitation. Excitation may be provided in the form of electromagnetic radiation (e.g., light). A reporter may also decrease or lose signal upon excitation. A signal emitted from a reporter (or detectable domain disposed thereon) may be detectable. A signal may be optical, chemical, radiometric, electronic, informational, or a combination thereof. An optical signal may be luminescent (e.g., chemiluminescent, bioluminescent, electroluminescent, sonoluminescent, photoluminescent, radioluminescent, or thermoluminescent. Some examples of photoluminescent optical signals include fluorescent or phosphorescent signals. An optical signal may come from a chromophore (e.g., a fluorophore, fluorescent dye). An optical signal may be any molecule, macromolecule, or molecular construct capable of emitting photons. Optical signals may be emitted in response to excitation. Optical signals may be differentiable from one another, such as by color. In some examples, it is advantageous to use multiple optical signals within a single system, method, or kit. For example, it may be advantageous to provide a plurality of fluorophores, some or all of which being capable of emitting a differentiable optical signal. A plurality of optical signals may include, for example, multiple colors. It may be advantageous to provide fluorescent dyes that produce one color, two colors, three colors, four colors, five colors, or more. It may be advantageous to provide fluorescent dyes that produce twenty colors or more. Fluorophores may include, for example, a fluorophore-iodoacetamide (e.g., Atto647N-Iodoacetamide); a fluorophore-succinimidyl ester (e.g., Atto647N-NHS), a fluorophore-amine (e.g., Atto6-(7N-Amine), a dithiolane-fluorophore (e.g. a custom synthesized fluorophore, an oxidized dithiolane-fluorophore, a reduce dithiolane-fluorophore), a fluorophore-Azide (e.g., Atto647N-Azide), Oregon Green (OG)-iodoacetamide, OG488-NHS, OG488-Azide, OG488-Tetrazine, OG514-NHS, Janelia Fluor (JF)-NHS, JF-FreeAcid, JF-Azide, JF-Dithiolane, Atto647N-Alkyne, Atto647N-FreeAcid, Atto425-NHS, Atto425-FreeAcid, Atto425-Amine, Atto425-Azide, Atto425-DBCO, SF554-NHS, or TexasRed-NHS. Optical signals may also comprise an absence or a loss of an optical signal (e.g., photobleaching, photoquenching) or a change in optical signal (e.g., FRET, BRET, homo-FRET, or other energy transfer luminescence).

A reporter may also contain a spacer. In some examples, a spacer adjoins a reporter and a signal-emitting entity. A spacer may adjoin two domains (e.g., a coupling domain and a detectable domain). A reporter may adjoin a second reactive group and a fluorescent dye, either directly or indirectly. A spacer may position two entities of interest at a specific distance from one another in order to optimize a functionality (e.g. FRET). A spacer may be implemented to prevent crowding or steric interference. A spacer may enhance the detectability of a signal. In some examples, a spacer enhances the reactivity of a second reactive group or a reactive (e.g. coupling) domain. A spacer can be composed of a polymer, a biopolymer, or a non-polymer, a heteroatomic chain, a polyamine chain, a polyester chain, a polyether chain, a polyamide chain

### Cleavable Unit

A cleavable unit may comprise functional groups, such as, for example, disulfides, A cleavable unit may be cleaved by, for example, enzymes, nucleophilic or basic reagents, reducing agents, photo-irradiation, electrophilic or acidic reagents, organometallic or metal reagents, oxidizing reagents, or combinations thereof. The cleavable group can be an acid cleavable aminomethyl group (e.g., rink-amide, Sieber, peptide amide linker (PAL)), hydroxymethyl (Wang-type), trityl or chlorotrityl, aryl-hydrazide linker. The cleavable group can be a metal cleavable group, such as, for example, an alloc linker, hydrazine cleavable group, or photo-labile cleavable group, such as, for example, nitrobenzyl based (e.g., 4-[4-(1-(Fmoc-amino)ethyl)-2-methoxy-5-nitrophenoxylbutanoic acid) or a carbonyl-based linker. The cleavable unit may be cleaved with TFA.

### Sample Types

The methods described herein may comprise analyzing a biological sample. A biological sample may be derived from a subject (e.g., a patient or a participant in a study), from a tissue sample (e.g., an engineered tissue sample), from a cell culture (e.g., a human cell line or a bacterial colony), from a cell (e.g., a cell isolated during a single cell sorting assay), or a portion thereof (e.g., an organelle from a cell or an exosome from a blood sample). A biological sample may be synthetic, such as a composition comprising of chemically synthesized peptides. A sample may comprise a single species or a mixture of species. A biological sample may comprise biomaterial from a single organism, from a colony of genetically near-identical organisms, or from multiple organisms (e.g., enterocytes and microbiota from a human digestive tract). A biological sample may be fractionated (e.g., plasma separated from whole blood), filtered, or depleted (e.g., high abundance proteins such as albumin and ceruloplasmin removed from plasma).

A sample may comprise all or a subset of the biomolecules from the subject, tissue sample, cell culture, cell, or portion thereof. For example, a sample from a subject may comprise the majority of proteins present in that subject, or may comprise a small subset of the proteins from that subject. A biological sample may comprise a bodily fluid such as cerebral spinal fluid, saliva, urine, tears, blood, plasma, serum, breast aspirate, prostate fluid, seminal fluid, stool, amniotic fluid, intraocular fluid, mucous, or any combination thereof. A biological sample may comprise a tissue culture, for example a tumor sample, or tissue from a kidney, liver, lung, pancreas, stomach, intestine, bladder, ovary, testis, skin, colorectal, breast, brain, esophagus, placenta, or prostate.

The biological sample may comprise a molecule whose presence or absence may be measured or identified. The biological sample may comprise a macromolecule, such as, for example, a polypeptide or a protein. The macromolecule may be isolated (e.g., separated from other components from which it was sourced) or purified, such that the macromolecule comprises at least 0.5%, at least 1%, at least 2%, at least 3%, at least 4%, at least 5%, at least 7.5%, at least 10%, at least 15%, at least 20%, at least 25%, at least 30%, at least 40%, at least 50%, at least 60%, at least 70%, at least 75%, at least 80%, at least 90%, at least 95%, at least 98%, or at least 99% of a composition by weight (e.g., by dry weight or including solvent). The biological sample may be complex, and may comprise a plurality of components (e.g., different polypeptides, heterogenous sample from a CSF of a proteopathy patient). The biological sample may comprise a component of a cell or tissue, a cell or tissue extract, or a fractionated lysate thereof. The biological sample may be substantially purified to contain molecules of a single type (peptides, nucleic acids, lipids, small molecules). A biological sample may comprise a plurality of peptides configured for a method of the present disclosure (e.g., digestion, C-terminal labeling, or fluorosequencing).

Methods consistent with the present disclosure may comprise isolating, enriching, or purifying a biomolecule, biomacromolecular structure (e.g., an organelle or a ribosome), a cell, or tissue from a biological sample. A method may utilize a biological sample as a source for a biological species of interest. For example, an assay may derive a protein, such as alpha synuclein, a cell, such as a circulating tumor cell (CTC), or a nucleic acid, such as cell-free DNA, from a blood or plasma sample. A method may derive multiple, distinct biological species from a biological sample, such as two separate types of cells. In such cases, the distinct biological species may be separated for different analyses (e.g., CTC lysate and buffycoat proteins may be partitioned and separately analyzed) or pooled for common analysis. A biological species may be homogenized, fragmented, or lysed prior to analysis. In particular instances, a species or plurality of species from among the homogenate, fragmentation products, or lysate may be collected for analysis. For example, a method may comprise collecting circulating tumor cells during a liquid biopsy, optionally isolating individual circulating tumor cells, lysing the circulating tumor cells, isolating peptides from the resulting lysate, and analyzing the peptides by a fluorosequencing method of the present disclosure. A method may comprise capturing peptides from a sample using a C-terminal capture reagent, and analyzing the peptides (e.g., by a fluorosequencing method).

Methods consistent with the present disclosure may comprise nucleic acid analysis, such as sequencing, southern blot, or epigenetic analysis. Nucleic acid analysis may be performed in parallel with a second analytical method, such as a fluorosequencing method of the present disclosure. The nucleic acid and the subject of the second analytical method may be derived from the same subject or the same sample. For example, a method may comprise collecting cell free DNA and a peptides from a human plasma sample, sequencing the cell free DNA (e.g., to identify a cancer marker), and performing proteomic analysis on the plasma proteins.

### Computer Systems

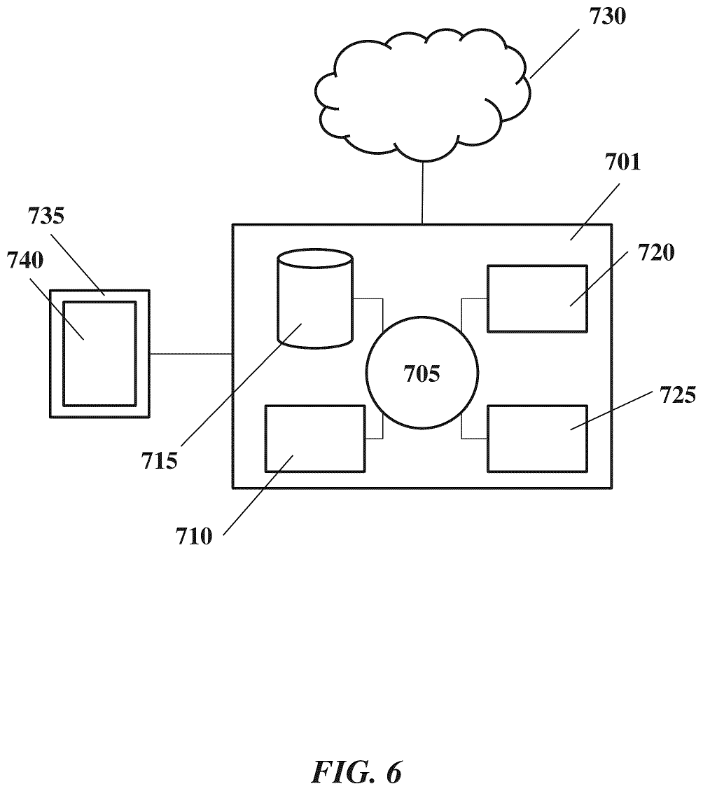

The present disclosure provides computer systems that are programmed to implement methods of the disclosure. FIG. 6 shows a computer system 601 that is programmed or otherwise configured to implement methods or parts of methods provided herein. The computer system 601 can regulate various aspects of the present disclosure, such as, for example, controlling reactions or reaction cycles (e.g., by adding reagents, adjusting temperature), analyzing signals from a system (e.g., fluorescent signals from a reporter moiety). The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user (e.g., a fluorescence spectrometer). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 630.

Methods as described herein can be implemented by way of machine (e.g., computer processor) executable code stored on an electronic storage location of the computer system 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, options for manipulating a reactor system. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, implement parts of methods described herein.

### EXAMPLES

### Example 1: Peptide Sample Preparation

### N-Terminal Immobilization of Peptides to a Support

The systems, methods, and kits provided herein describe various aspects of attaching a peptide, to be processed or analyzed, to a support. In this example, a peptide is to be attached to a bead in order to facilitate analysis of the peptide. The beads in this example can be functionalized beads, appended with a cleavable linker and a capture reagent. The capture reagent can be a pyridine carboxaldehyde (PCA), which can covalently couple to the N-terminus of a peptide. At the end of the procedure, the peptides can be scarlessly removed by use of a derivatized hydrazine reagent. The derivatized hydrazine reagent is 2-(dimethylamino)ethylhydrazine dihydrochloride (CAS No. 57659-80-0). It may be advantageous to digest a larger peptide or protein using a protease such as GluC or trypsin in order to isolate peptides of a smaller size, which will facilitate quick, efficient, and accurate processing and analysis (e.g., sequencing). However, large or full-length peptides (e.g. proteins) may also be processed and analyzed. It may also be advantageous to provide peptides in a buffered solution (e.g., a HEPES buffer, pH 8.0).

As illustrated in FIG. 2 the first step is to capture a peptide (e.g., a plurality of peptides in a fluid sample) on the PCA-functionalized bead in order to immobilize the peptide. The PCA moiety reacts specifically with the N-terminal amine in a peptide. Through an intramolecular cyclization with the adjacent amide bond, the PCA capture agent reacts with the N-terminus over other amines (e.g., lysine) in the peptide, ensuring the desired attachment. In a subsequent step, the immobilized peptide is labeled, cleaved from the bead, and analyzed via fluorosequencing. The PCA beads are a derivatized rink resin comprising a polymeric PEG bead, a spacer, a rink moiety, and the capture agent (PCA) linked in a linear arrangement (as shown in Scheme 1). Additional reagents of utility in preparing the immobilized peptides include HEPES buffer (pH 8.0), dimethylformamide (DMF), water, borosilicate glass culture test tubes, and biospin chromatography column (Biorad, catalog #732-6008).

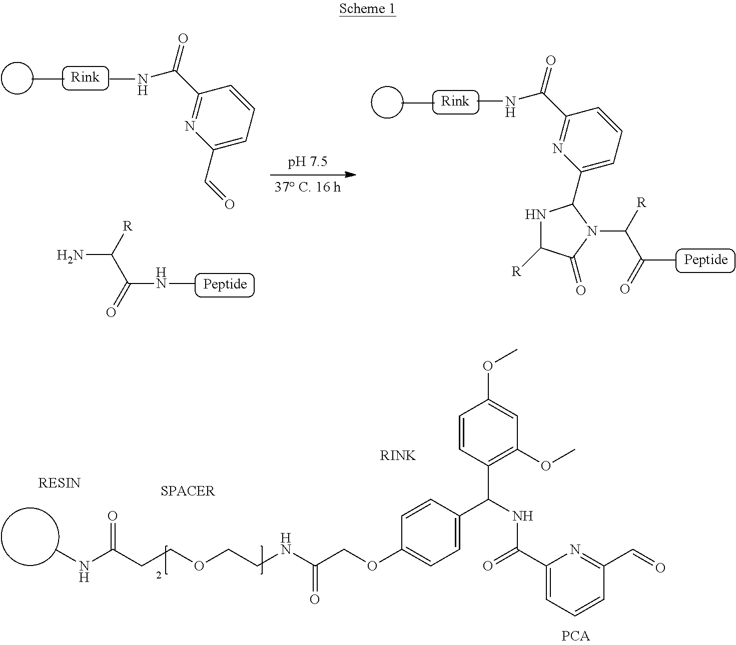

Conditions include the following: (i) approximately 250 microliters (μL) of beads are added to the biospin chromatography column, an amount that is sufficient for approximately 1 milligram (mg) of peptide, (ii) the column is placed on a test-tube (13 mm×100 mm), (iii) wash (see Protocol (A) below); (iv) peptides are solubilized in 1 milliliter (mL) HEPES buffer (pH 8.0), (v) cap the spin column and add peptide solution; (vi) incubate the column at 37° C. in a shaker overnight, (vii) wash (see Protocol (B) below), This procedure results in immobilization of the peptide on a support (e.g., bead). A pH between 7 and 9 is preferred, as an acidic pH may not allow the immobilization reaction to occur.

### Wash Protocols

A solid support system exemplified for use in the systems, methods, and kits described herein is a PEG based polymeric bead. While any support(s) disclosed herein may be used, for brevity, only the PEG based polymeric bead is described. The PEG beads is functionalized with PCA, then peptides immobilized thereto. The solid-phase capture allows for washes of the beads to (a) remove previous reagents, dyes, salts, etc., and (b) provide an environment for labeling of peptides without loss of the substrates. These beads may be sensitive and may crack when desiccated. Beads are stored in DMF and methanol, which will facilitate the expansion and suspension of the beads. Washes of the beads is done sequentially, moving from organic conditions to aqueous buffers, and in some conditions, back to organic conditions from aqueous buffers. Materials and reagents used in washing steps include dimethylformamide (DMF), acetonitrile, methanol, water, and borosilicate glass culture test tubes (100 mm×13 mm).

The washing steps, as may be required in various examples, are performed as follows: Remove biospin column mounted on the borosilicate glass test tube. Next, the sample within the biospin column is washed according to either protocol (a), to transition from aqueous to organic conditions, or (b), to transition from organic to aqueous conditions.

Protocol (A) aqueous to organic: wash the column (i) twice with water, then perform (ii) at last one wash with (1:1 v/v) water/acetonitrile, followed by (iii) at least two washes with (1:1 v/v) acetonitrile/DMF. Between each wash, the beads are incubated in the solvent for approximately 10 minutes.

Protocol (B) organic to aqueous: (0) if methanol is used, first wash the column with methanol once before proceeding to (i) two washes with DMF, followed by (ii) at least one wash with acetonitrile, followed by (iii) at least one wash with (1:1 v/v) acetonitrile/water, followed by (iv) two washes with water. Between each wash, the beads incubate in the solvent for approximately 10 minutes.

### Example 2: Peptide Labeling

### C-Terminal Labeling

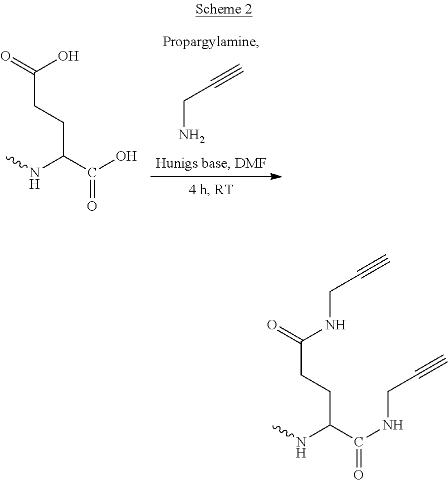

If a peptide is digested using GluC under pH 8 digestion buffer, or a sufficiently similar protease/buffer system, the cleavage site occurs on the C-terminus of an acidic residue (e.g., aspartic acid and glutamic acid) (Scheme 2). Thus, with all of the acidic residues of the peptide at the C-terminus of the peptides, the peptide is suitable for amide coupling. The C-terminal acidic residue(s) are converted to alkynes for subsequent immobilization to a support. Whether the C-terminal carboxylic acid, the side chain carboxylic acid, or both are alkynylated and immobilized to the support may not affect the function of the systems, methods, and kits as disclosed herein. Alternate reactive groups can be used in place of an alkyne. However, for brevity, only the alkyne example is discussed herein.

First, the previously described peptide is immobilized to a first support (e.g., a functionalized bead) is provided in a quantity of less than 1 micromole. Additional reagents of utility for C-terminal labeling include propargylamine (CAS No. 2450-71-7), 0-(1H-6-Chlorobenzotriazole-1-yl)-1,1,3,3-tetramethyluronium hexafluorophosphate (HCTU) (CAS No. 330645-87-9), N,N-Diisopropylethylamine (Hunig's base, DIEA, or DIPEA) (CAS No. 7087-68-5) stock concentration 5.7 M, and dimethylformamide (DMF) (CAS No. 68-12-2).

The immobilized peptides are washed with one column volume of DMF. A reaction is then prepared with the following (or substantially similar) composition: (a) HCTU—20 mg, 50 eq; (b) Hunigs base—8 μL, 50 eq; (c) propargylamine—3 μL, 50 eq; and (d) dimethylformamide 500 μL. The biospin reactor is capped and the mixture added to the peptide. The contents are then incubated at ambient temperature for 1-4 h with constant mixing. The contents are then washed following the previously prescribed wash steps (e.g., protocol (b) then protocol (a)), storing the contents in DMF until the next step is executed. The resultant product is a peptide immobilized to a support (e.g., a PCA-functionalized bead) at its N-terminus, featuring an alkynylated C-terminus and acidic C-terminal amino acid (e.g., alkynylated glutamic acid or aspartic acid).

For methods of immobilizing fluorescently labeled peptides on a functionalized glass slide, click chemistry is used. Approximately 200 pM of peptides were immobilized on an azide-silane slide (custom slides from PolyAn, Germany) using standard Cu(I)-Click chemistry. Briefly, a 2 mL solution comprising peptide (200 pM), CuSO₄/tris-hydroxypropyltriazolylmethylamine (THPTA) mix (1 mM/0.5 mM) and freshly prepared sodium L-ascorbate (5 mM) was incubated on the azide-silane slide at room temperature for 2 hours. Following the incubation, the slides were rinsed with water and fluorosequencing performed as previously described⁶ with minor modifications. To deprotect the N-terminal PCA cap, the slides were bathed in 0.5 M DMAEH at 60° C. for 16 hours. Deprotection of the Fmoc group was performed by incubating slides with 20% Piperidine solution in DMF for 1 hour. The images were processed using custom developed scripts.

### Cysteine Labeling

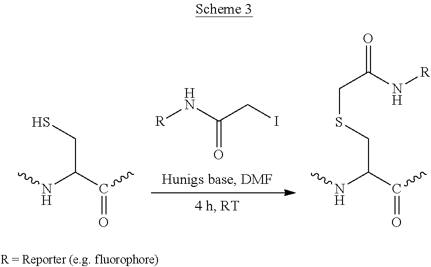

The free thiol group of a cysteine side chain is nucleophilic and may risk side-reactions in later steps (Scheme 3). To control this, the side chain thiol is reacted with a second agent, thereby reducing the risk of side-reactions or cross-reactivity. Specifically, the thiol is reacted with an inert label (e.g., protecting group), a functional label (e.g., one with a reactive group that can couple to another entity), or a reporter label (e.g., a label that emits a signal).

In order to label a cysteine with an iodoacetamide functionalized fluorophore (reporter capable of emitting a signal upon excitation), the following reagents are of utility: fluorophore-iodoacetamide (e.g., Atto647N-iodoacetamide)—200 μg in 20 μL of DMF stock solution, DMF, water, and iodoacetamide (e.g., as a non-reporter “dummy” label).

The immobilized peptides are first washed with one column volume of freshly distilled DMF. A reaction is then prepared with the following (or substantially similar) composition: (a) fluorophore-iodoacetamide stock solution 200 ug in 20 uL; 0.05 mM (50 PM) in 500 μL; 0.2 umol; and (b) DMF—500 μL. The biospin reactor is capped and the mixture added to the peptide. The contents are then incubated at ambient temperature for 2 h with constant mixing. The contents are then washed following the previously prescribed wash steps (e.g., protocol (b) then protocol (a)), storing the contents in DMF until the next step is executed. The resultant product is a peptide wherein the cysteine residues are labeled with an optionally fluorophore-coupled iodoacetamide label. As described previously, alternate labels may be used in a similar manner to that described herein.

### Lysine Labeling

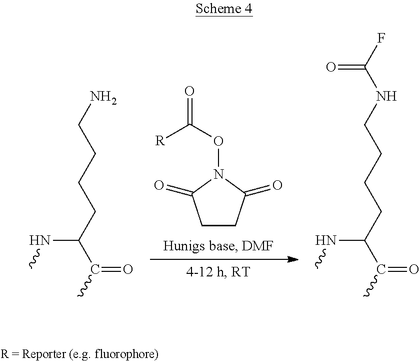

The amine of a lysine can be labeled specifically with an NHS ester (Scheme 4). Because the N-terminal primary amine is prior-coupled to a support, the lysine amino acids is the only entity with free primary amines. This step may be performed after cysteine labeling in order to prevent cross-reactivity.

In order to label a lysine with an NHS ester functionalized fluorophore (reporter capable of emitting a signal upon excitation), the following reagents are of utility: fluorophore-succinimidyl ester (e.g., Atto647N-NHS)—200 μg in 20 μL of DMF stock solution, N,N-diisopropylethylamine (Hunig's base), anhydrous DMF, and sulfo-NHS acetate (e.g., as a non-reporter “dummy” label).

The immobilized peptides are washed with one column volume of DMF. A reaction is then prepared with the following (or substantially similar) composition: (a) fluorophore-NHS stock solution 200 ug in 20 uL; 0.05 mM (50 μL) in 500 μL; 0.2 umol; (b) 1 mM solution of Hunig's base—1 μL; and (c) anhydrous DMF—500 μL. The biospin reactor is capped and the mixture added to the peptide. The contents are then incubated at ambient temperature for 2 h with constant mixing. The contents are washed following the previously prescribed wash steps (e.g., protocol (b) then protocol (a)), storing the contents in DMF until the next step is executed. The resultant product is a peptide wherein the lysine residues will be labeled with an optionally fluorophore-coupled NHS ester label. As described previously, alternate labels may be used in a similar manner to that described herein.

### Aspartic Acid/Glutamic Acid Labeling

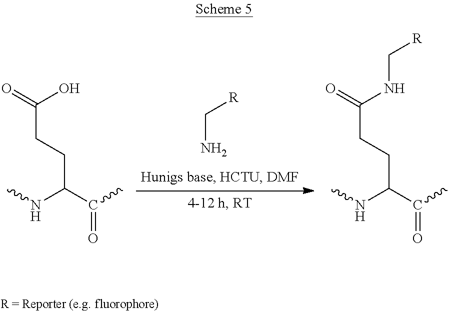

The carboxylic acid of aspartic acid and glutamic acid are labeled through a direct amide coupling step with an amine functionalized reagent (Scheme 5). This step can apply to internal acidic residues. In the example where GluC is to be used as a protease, all acidic residues will be C-terminal and have been previously alkynylated as described above in the C-terminal labeling section. Excluding use of GluC, internal acidic residues are labeled as described herein subsequent to labeling of lysine residues, in order to prevent intramolecular cyclization or cross-reaction.

In order to label an aspartic acid or glutamic acid with an amine functionalized fluorophore (reporter capable of emitting a signal upon excitation), the following reagents are of utility: fluorophore-amine ester (e.g., Atto647N-Amine)—200 μg in 20 μL of DMF stock solution, N,N-diisopropylethylamine (Hunig's base), O-(1H-6-Chlorobenzotriazole-1-yl)-1,1,3,3-tetramethyluronium hexafluorophosphate (HCTU), anhydrous DMF, and propylamine (e.g., as a non-reporter “dummy” label). Alternate bases, coupling reagents, or labels may be used analogously to the procedure described herein.

The immobilized peptides are washed with one column volume of freshly distilled DMF. A reaction is then be prepared with the following (or substantially similar) composition: (a) fluorophore-NH2 stock solution 200 ug in 20 uL; 0.05 mM (50 μL) in 500 μL; 0.2 umol; (b) 500 mM solution of Hunig's base—1 μL; (c) HCTU—2 mg, 5 eq; and (c) anhydrous DMF—500 μL. The biospin reactor is capped and the mixture added to the peptide. The contents are incubated at ambient temperature for 2 h with constant mixing. The contents are washed following the previously prescribed wash steps (e.g., protocol (b) then protocol (a)), storing the contents in DMF until the next step is executed. The resultant product is a peptide wherein the aspartic acid and glutamic acid residues will be labeled with an optionally fluorophore-coupled amine label. As described previously, alternate labels may be used in a similar manner to that described herein.

### Tyrosine Labeling

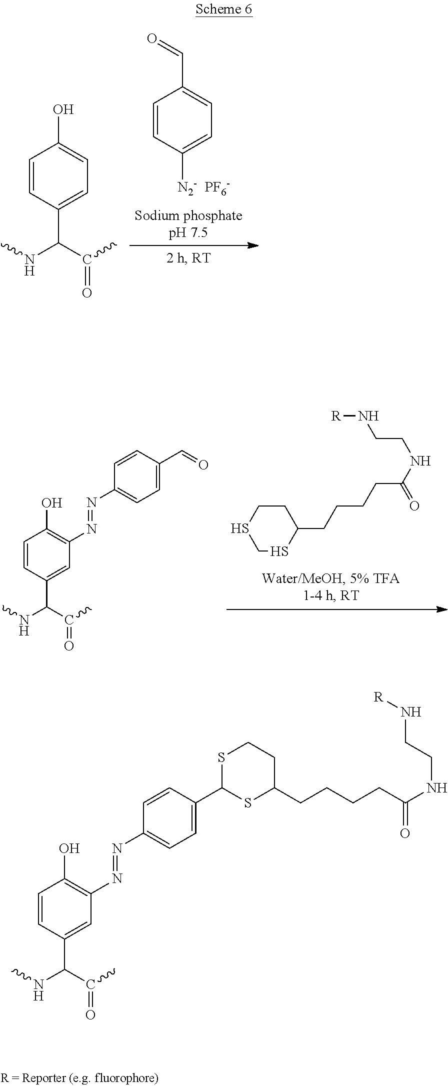

The position adjacent (e.g. ortho to) the phenolic moiety of tyrosine can be labeled through a two-step labeling process using a bifunctional diazonium reagent (Scheme 6). The diazonium salt can functionally couple for approximately 3-4 weeks after its preparation, but may demonstrate diminished activity thereafter. Freshly prepared reagent may be preferred for use herein. A second reagent, a dithiolane-fluorophore (or dithiolane non-fluorophore), is provided in order to label one or more tyrosine residues within a peptide. Either reagent may be custom synthesized or ordered from an appropriate vendor, if available.

In order to label a tyrosine with a functionalized dithiolane fluorophore (reporter capable of emitting a signal upon excitation), the following reagents are of utility: sodium phosphate buffer—150 mM or greater, pH 7.5; trifluoroacetic acid; methanol; water; diazonium salt stock (e.g., custom synthesized)—1 mg in 25 μL DMF fresh; dithiolane-fluorophore (e.g., custom synthesized)—200 μg in 20 μL DMF fresh; DMF; and dithiolane-alkyl (e.g., as a non-reporter “dummy” label). The diazonium salt used in this example is 4-formylbenzenediazonium hexafluorophosphate. One should note that part of this labeling step may take place prior to immobilization of the peptide to a support.

A reaction is first be prepared with the following (or substantially similar) composition: (a) peptides solubilized in 50-100 μL sodium phosphate buffer; (b) 4-formylbenzenediazonium hexafluorophosphate salt—1 mg in 25 μL DMF, 4 eq; and (c) 150 mM sodium phosphate buffer, pH 7.5-50-100 μL. The reaction vessel is capped, and the mixture is incubated at ambient temperature for 2 h with constant mixing. The sample is diluted with DMF to 1 mL total volume. Next, the peptide is immobilized to a support as described previously in the section titled, “Immobilization of Peptides to a Support.” A methanolic acidic solution is prepared, containing the following reagents: (i) 475 μL methanol; (ii) 475 μL water; and (iii) 50 μL trifluoroacetic acid. The dithiolane-fluorophore or dithiolane-alkyl dummy (200 μg, reduced with TCEP) are solubilized in 500 μL of the methanolic acidic buffer and incubated with the immobilized peptide for 2 h. Finally, the column is washed with 10 column volumes of water, followed by the washing steps (protocol (a)) as previously described, storing the contents in DMF until the next step is executed. The resultant product is a peptide wherein the tyrosine residues will be labeled with an optionally fluorophore-coupled dithiolane label. As described previously, alternate labels may be used in a similar manner to that described herein.

### Histidine Labeling

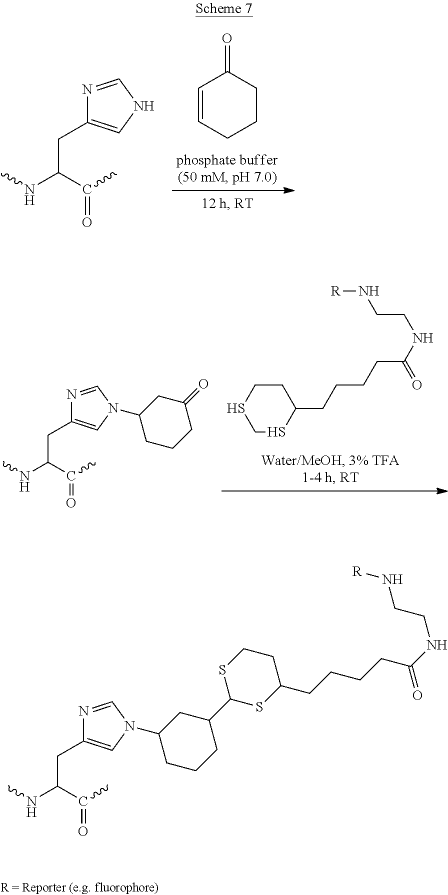

The secondary amine of histidine can be selectively labeled through a two-step labeling process using a cyclohexenone reagent (Scheme 7). 2-cyclohexenone is reacted with histidine in a nucleophilic addition reaction, after which the ketone is reacted to append a reporter (or non-reporter label). One reagent capable of reacting with the ketone is a dithiolane as described previously. A derivatized version with a different fluorescent moiety to the one described for tyrosine labeling may provide additional utility, enabling one to differentiate the two amino acids based on the different reporters. Conversely, the specificity of the labeling chemistry (e.g., from the reactivity of the diazonium vs. the cyclohexanone) can be used to identify which dithiolane corresponds to which amino acid.

In order to label a histidine with a functionalized dithiolane fluorophore (reporter capable of emitting a signal upon excitation), the following reagents are of utility: phosphate buffer—50 mM or greater, pH 7.5; 2-cyclohexenone; trifluoroacetic acid; methanol; water; dithiolane-fluorophore (e.g., custom synthesized)—200 μg in 20 μL DMF fresh; DMF; and dithiolane-alkyl (e.g., as a non-reporter “dummy” label).

First, the peptide immobilized to a support is equilibrated in phosphate buffer, preceded by washing steps as described above if necessary. Next, a solution of phosphate buffer (500 μL) and cyclohexenone (25 μL; 50 eq) is added to the biospin column containing the immobilized peptide. The reaction vessel is capped, and the mixture is incubated at ambient temperature or 30° C. for 12 h with constant mixing. The contents of the reaction are washed, following protocol (a) as provided above, ultimately equilibrating in methanol. A methanolic acidic solution are prepared, containing the following reagents: (i) 475 μL methanol; (ii) 475 μL water; and (iii) 50 μL trifluoroacetic acid. The dithiolane-fluorophore or dithiolane-alkyl dummy (200 μg, reduced with TCEP) are solubilized in 500 μL of the methanolic acidic buffer and incubated with the immobilized peptide for 2 h. Finally, the column is washed with 10 column volumes of water, followed by the washing steps (protocol (a)) as previously described, storing the contents in DMF until the next step is executed. The resultant product is a peptide wherein the histidine residues will be labeled with an optionally fluorophore-coupled dithiolane label. As described previously, alternate labels may be used in a similar manner to that described herein.

### Arginine Labeling

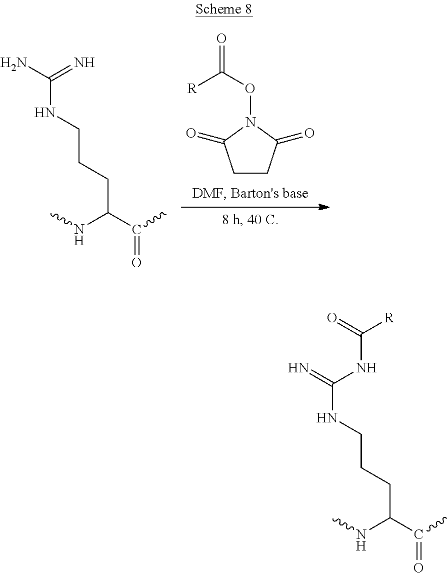

The amine of an arginine residue can be labeled specifically with an NHS ester with the aid of Barton's base (Scheme 8). The reaction provided herein may show cross-reactivity or interference by primary amines (e.g., N-terminus, lysine) or thiols (e.g., cysteine). This step may be performed after N-terminal support immobilization and cysteine and lysine labeling in order to prevent cross-reactivity.

In order to label an arginine with an NHS ester functionalized fluorophore (reporter capable of emitting a signal upon excitation), the following reagents are of utility: fluorophore-succinimidyl ester (e.g., Atto647N-NHS)—200 μg in 20 μL of DMF stock solution, 2-tert-butyl-1,1,3,3-tetramethylguanidine (Barton's base), anhydrous DMF, and sulfo-NHS acetate (e.g., as a non-reporter “dummy” label).

The immobilized peptides are washed with one column volume of DMF. A reaction is prepared with the following (or substantially similar) composition: (a) Barton's base—2 μL; and (b) anhydrous DMF—200 μL; followed by (c) fluorophore-NHS stock solution 200 ug in 20 uL; 0.05 mM (50 pM) in 500 μL; 0.2 umol; The biospin reactor is capped and the mixture added to the peptide. The contents are incubated at 40° C. for 8 h with constant mixing. The contents are washed following the previously prescribed wash steps (e.g., protocol (b) then protocol (a)), storing the contents in DMF until the next step is executed. The resultant product is a peptide wherein the arginine residues will be labeled with an optionally fluorophore-coupled NHS ester label. As described previously, alternate labels may be used in a similar manner to that described herein.

### Methionine Labeling

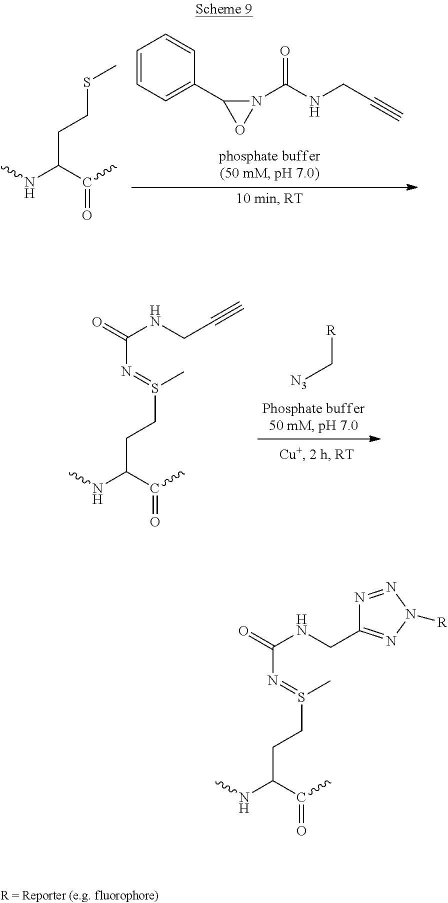

Methionine has weak nucleophilicity and can be selectively labeled by a redox based scheme where an oxaziridine group reacts specifically with the thioether without cross-reacting with cysteine (Scheme 9). The bond formed is stable to reducing agents such as TCEP.

In order to label a methionine with an azide functionalized fluorophore (reporter capable of emitting a signal upon excitation), the following reagents are of utility: fluorophore-azide (e.g., Atto647N-Azide)—200 μg in 20 μL of DMF stock solution; sodium L-ascorbate—10 mg in 250 μL buffer (200 mM); tris(hydroxypropyltriazolylmethyl) (THPTA)—8.7 mg in 200 μL water (200 mM); copper sulphate—4 mg in 120 μL water, (200 mM); water; phosphate buffer—pH 7 (50 mM); and oxaziridine reagent, depicted in the scheme above.

First the oxaziridine reagent is prepared by diluting 5 μL of the concentrated reagent in 500 μL phosphate buffer. The immobilized peptides are incubated with the oxaziridine reagent at ambient temperature for 10 min. The immobilized peptide is washed with buffer and water or, if necessary, following the washing steps previously outlined. A 100 mM stock solution of copper sulphate reagent is prepared by combining CuSO₄ (50 μL), THPTA (25 μL), and water (25 μL); incubate with peptide 30 min. A reaction is prepared with the following (or substantially similar) composition: (a) phosphate buffer (400 μL); (b) sodium ascorbate (25 μL) (c) CuSO₄ stock solution (50 μL); and (d) fluorophore-azide stock solution 200 μg in 20 μL; 0.05 mM (50 PM) in 500 μL; 0.2 μmol. The reaction is combined with the immobilized peptide, and the contents will then be incubated at ambient temperature for 2-4 h with constant mixing. The contents are washed following the previously prescribed wash steps (e.g., protocol (a) then protocol (b)), storing the contents in DMF until the next step is executed. The resultant product is a peptide wherein the methionine residues will be labeled with an optionally fluorophore-coupled triazole label. As described previously, alternate labels may be used in a similar manner to that described herein.

### Labeling Phosphorylated Residues

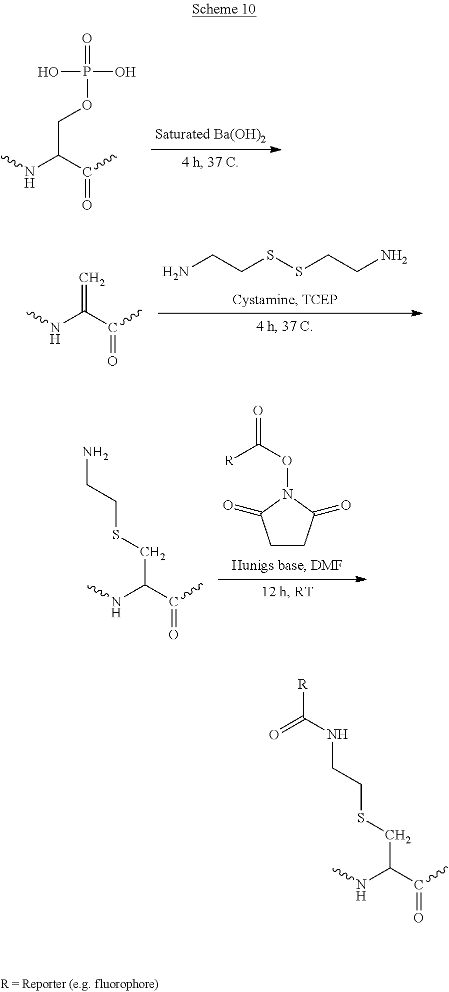

Phosphorylated amino acids such as serine and threonine can be labeled through beta-elimination followed by conjugate addition (e.g., a Michael acceptor reaction) (Scheme 10). This series of reaction steps provides selective labeling of phosphorylated serine (pSer) and threonine (pThr) over other phosphorylated amino acids such as tyrosine (pTyr). A subsequent pan-phospho labeling method can be implemented to label pTyr.

In order to label a pSer and pThr with a functionalized NHS ester fluorophore (reporter capable of emitting a signal upon excitation), the following reagents are of utility: solubilized peptide—1 umol in 100 uL water or water/acetonitrile (1:1 vv); barium hydroxide, octahydrate (46 mg in 1 mL water); HEPES buffer—pH 8.5 (100 mM); Tris(2-carboyethyl)phosphine hydrochloride solution (TCEP) (500 mM); cystamine dihydrochloride—152 mg in 1 mL water (1 M); fluorophore-succinimidyl ester (e.g., Atto647N-NHS)—200 ug in 20 uL of DMF stock solution; Hunig's base (500 mM in DMF); and anhydrous DMF.

A first reaction is prepared in a microfuge vial by combining 100 μL of peptide solution (1 umol) with 100 μL of barium hydroxide solution. The reaction mixture is incubated at 37° C. for 4 h, followed by centrifugation at 5000 rpm for 2 min. Barium phosphate precipitates out of solution. The contents are neutralized with HEPES buffer, and are immobilized to a support, as described previously. Next, a second reaction is prepared with the following composition: (a) 1 M cystamine solution (50 uL); (b) 500 mM TCEP solution (100 uL); (c) HEPES buffer (100 uL); and (d) water (250 uL). The reaction mixture is incubated for 4-12 h at 37° C. with constant mixing. After reaction completion, the reaction is washed with 10 column volumes of water, followed by washing protocol (a). Next, a third reaction is prepared with the following composition: fluorophore-NHS, 200 ug in 20 uL, 0.5 mM (500 uM) in 500 uL. 0.2 umol; 500 mM stock solution Hunig's base (1 μL); and anhydrous DMF (500 μL). The reaction mixture is added to the immobilized peptide, and the reaction vessel is capped and incubated at ambient temperature for 4 h with constant mixing. After completion of the reaction, the peptide is washed following the previously described washing steps (protocol (b) followed by protocol (a)), storing the contents in DMF until the next step is executed. The resultant product is a peptide wherein the pSer and pThr residues will be labeled with an optionally fluorophore-coupled label. As described previously, alternate labels may be used in a similar manner to that described herein.

### Example 3: Peptide Cleavage from a Support

A peptide is cleaved from a support following a two-step procedure involving trifluoroacetic acid (TFA) followed by hydrazine. In the example where the support is a PCA resin comprising a bead, a spacer, a rink moiety, and a PCA moiety bound to a peptide, the PCA moiety is cleaved from the rink moiety by treatment with TFA. In a second step, the peptide is cleaved from the PCA moiety, thereby providing a peptide with an intact N-terminus.

In order to cleave the peptide from a support such as a PCA resin, the following reagents are of utility: immobilized peptide, optionally labeled (e.g., with one or more fluorophore labels); 500 mM Hunig's base; DMF; methanol; water; 100 mM dimethylaminoethylhydrazine dihydrochloride (DMEAE-hydrazine)—440 mg in 25 mL PBS; acetonitrile; trifluoroacetic acid; triisopropylsilane; dichloromethane (DCM); and diethylether (Et₂O).

First, the immobilized peptide is washed according to the previously described washing steps following protocol (a) or, if stored in organic medium previously, protocol (b) followed by protocol (a). The peptide is washed extensively with DCM, then dried under vacuum. Next, a TFA solution is prepared with the following (or substantially similar) composition: (a) TFA (4.8 mL); (b) triisopropylsilane (0.1 mL); and (c) water (0.1 mL). The TFA solution (1 mL) is added to the peptide, and the reaction vessel is capped and spun. The reaction is incubated at ambient temperature or slightly warmer (e.g., 30° C.) for 1-2 h before eluting the solution into a microcentrifuge tube. TFA is removed or substantially decreased in volume with the aid of nitrogen purging. The peptide is resuspended in Et₂O and cooled to −80° C. The microcentrifuge tube is centrifuged at 4° C. at 10,000 rpm for 10 min. After removing the supernatant, the pellet is solubilized with 1:1 v/v methanol/water. Next, the solubilized peptide is immobilized to a second support (e.g., a glass slide) as described below. Once the peptide is immobilized to a second support (e.g., through the peptide's C-terminal end), the PCA moiety can be scarlessly cleaved from the N-terminus with the aid of hydrazine. As outlined in the reagents list above, a hydrazine solution is prepared using 440 mg DMEAE-hydrazine and 25 mL PBS buffer. The peptide immobilized to the second support is soaked in the hydrazine solution, and the solution is heated to 60° C. for 12-16 h. The peptide is washed with water. The end result is a labeled peptide immobilized to a second support at its C-terminus, facilitating fluorosequencing (e.g., using Edman degradation or related techniques) from the N-terminus.

### Example 4: Peptide Capture Procedure

This example covers a method for selectively capturing peptides on a lantern. The present disclosure provides a range of substrates for capturing peptides. One such type of substrate is a lantern, which may comprise a solid support comprising peptide capture agents, and a rod for positioning the solid support within a sample. A lantern rod may be manipulatable by a user (e.g., the user may hold the lantern rod) or an instrument. A lantern solid support may comprise a reactive group of the present disclosure, such as a reactive group selective for cysteine or a peptide C-terminus.

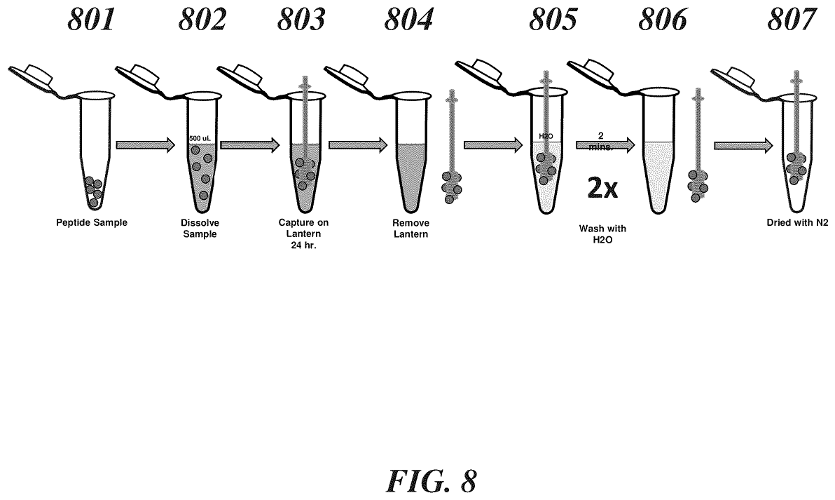

FIG. 8 outlines a method for using a lantern consistent with the present disclosure. A peptide mixture containing angiotensin (provided as a positive control peptide), a peptide comprising the sequence AKGAGRY{PRA}N-ONH₂ (SEQ ID NO: 4, where {PRA} denotes Propargylglycine), a capture negative control peptide, and a peptide of interest are dissolved in approximately 500 μL of a solution comprising water, 3% acetonitrile, and 0.1% formic acid. A lantern a solid support comprising peptide capture agents is placed in the sample and incubated for 24 hours at 37° C., providing sufficient time for angiotensin, 2K peptide, the capture negative control peptide, and the peptide of interest to couple to the peptide capture agents of the lantern. The lantern is then washed twice for two minutes in fresh deionized water to remove unbound peptides. The lantern is then dried in air or with an N₂ flow. Finally, the lantern is placed in a clean centrifuge tube for storage or shipping. The peptides coupled to the lantern may be recovered by resuspending the lantern in a solution and providing a cleavage agent to decouple the peptides from the peptide capture agent.

### Example 4: Fluorosequencing Assay Stability and Efficiency

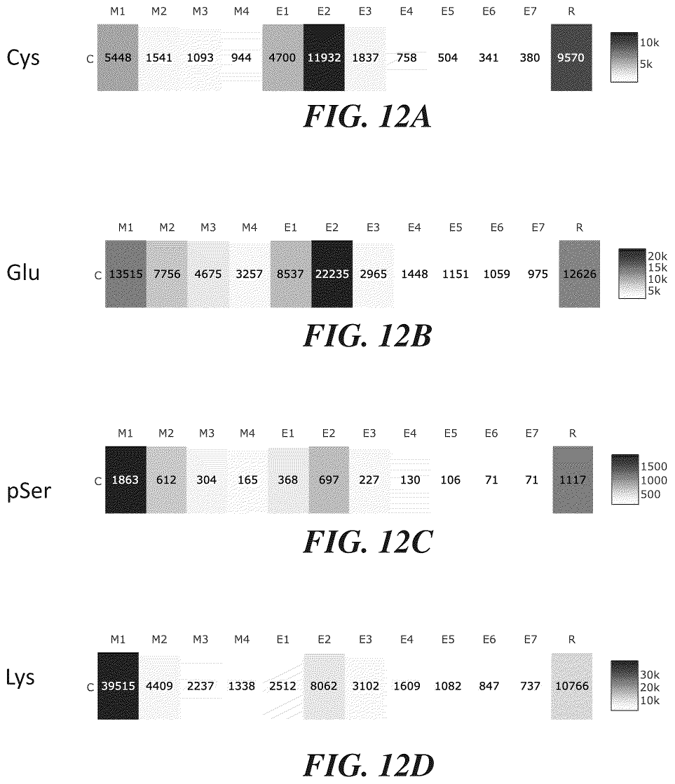

This example covers a set of example fluorosequencing assays. In this example, peptides of sequence AXAGANGSNG{PRA}N (SEQ ID NO: 5), wherein PRA is Propargylglycine and X is the amino acid—Cys, Glu, pSer, or Lys, as indicated in FIG. 12A-D. The Cys, Glu, pSer, or Lys at position X is reacted with a label are reacted with a “click” label comprising either an Azide second reactive group (FIG. 12A-C) or a Norbornene second reactive group (FIG. 12D). A “clack” reporter moiety is then coupled to the “click” label (the fluorophore Atto647N-PEG4-DBCO is coupled to the azide click handle and the fluorophore Atto647N-PEG4-mTET is coupled to the norbornene click handle).

FIG. 12A-D provide fluorosequencing results on the labeled peptides depicted as heat maps showing the counts of fluorescent spots that disappear after every Edman cycle (E). Control cycles that lack the Edman reagent is denoted as M. The maximum counts in E2 position. For cysteine, glutamate, and lysine (FIG. 12A, B, and D), the largest fluorescence decrease occurs after the second Edman degradation step. The results show that peptides can be stably labeled with “click” label-“clack” reporter moiety pairs.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-146. (canceled)

147. A system comprising a peptide, wherein said peptide is immobilized to at least one support; and wherein said peptide comprises an amino acid coupled to a label, wherein said label comprises (i) a first reactive group that is configured to couple to a second reactive group that is coupled to a first reporter moiety configured to emit at least a first signal or signal change, (ii) a third reactive group that is configured to couple to a fourth reactive group that is coupled to a first protecting group configured to prevent coupling between said third reactive group and a second reporter moiety, or (iii) a fifth reactive group that is coupled to a second protecting group configured to prevent coupling between said amino acid and a third reporter moiety.

148. The system of claim 147, wherein a C-terminus of said peptide is coupled to said at least one support.

149. The system of claim 148, wherein said C-terminus is modified with a compound comprising an alkyne or an azide.

150. The system of claim 147, wherein an N-terminus of said peptide is coupled to a first support of said at least one support, wherein said first support is a bead, or wherein a C-terminus of said peptide is coupled to a second support of said at least one support, wherein said second support is a microscopic slide.

151. The system of claim 147, wherein said amino acid is selected from the group consisting of lysine, cysteine, glutamic acid, aspartic acid, tyrosine, arginine, histidine, threonine, serine, proline, asparagine, glutamine, and tryptophan.

152. The system of claim 147, wherein said first reactive group or said third reactive group is selected from the group consisting of an azide, an alkyne, an alkene, an aldehyde, a ketone, a tetrazine, a thiol, a dithiol, a cyclooctene, and norbornene.

153. The system of claim 147, wherein said second reactive group or said fourth reactive group is selected from the group consisting of an alkyne, an azide, a thiol, a dithiol, a cyclooctene, an alkene, an aldehyde, a ketone, a tetrazine, and norbornene.

154. The system of claim 147, wherein:
(a) said first reactive group is the same as said third reactive group;
(b) said first reactive group is the same as said fifth reactive group;
(c) said third reactive group is the same as said fifth reactive group; or
(d) said second reactive group is the same as said fourth reactive group.

155. The system of claim 147, wherein
(a) said first reactive group is different from said third reactive group;
(b) said first reactive group is different from said fifth reactive group;
(c) said third reactive group is different from said fifth reactive group;
(d) said second reactive group is different from said fourth reactive group.

156. The system of claim 147, wherein:
(a) said first reporter moiety is the same as said second reporter moiety;
(b) said first reporter moiety is the same as said third reporter moiety;
(c) said second reporter moiety is the same as said third reporter moiety; or
(d) said first protecting group is the same as said second protecting group.

157. The system of claim 147, wherein
(a) said first reporter moiety is different from said second reporter moiety;
(b) said first reporter moiety is different from said third reporter moiety;
(c) said second reporter moiety is different from said third reporter moiety; or
(d) said first protecting group is different from said second protecting group.

158. A method for processing or analyzing a peptide, comprising:
(a) providing said peptide comprising an amino acid coupled to a label, wherein said label comprises (i) a first reactive group that is configured to couple to a second reactive group that is coupled to a first reporter moiety configured to emit at least a first signal or signal change, (ii) a third reactive group that is configured to couple to a fourth reactive group that is coupled a first protecting group configured to prevent coupling between said third reactive group and a second reporter moiety, or (iii) a fifth reactive group that is coupled to a second protecting group configured to prevent coupling between said amino acid and a third reporter moiety;
(b) bringing said peptide in contact with a mixture comprising (1) said second reactive group or (2) said fourth reactive group;
(c) with said peptide immobilized to at least one support, detecting at least a second signal or signal change from said peptide; and
(d) using said at least said second signal or signal change to identify said amino acid or an additional amino acid of said peptide.

159. The method of claim 158, wherein, in (a) or (b), said peptide is immobilized to said at least one support.

160. The method of claim 158, wherein said peptide comprises a plurality of amino acids.

161. The method of claim 160, wherein at least one amino acid of said plurality of amino acids is selected from the group consisting of lysine, cysteine, glutamic acid, aspartic acid, tyrosine, arginine, histidine, threonine, serine, asparagine, glutamine, and tryptophan.

162. The method of claim 158, wherein said label comprises said first reactive group that is configured to couple to said second reactive group that is coupled to said first reporter moiety configured to emit said at least said first signal or signal change.

163. The method of claim 158, wherein said label comprise said third reactive group that is configured to couple to said fourth reactive group that is coupled to said first protecting group configured to prevent coupling between said third reactive group and said second reporter moiety.

164. The method of claim 158, wherein said label comprises said fifth reactive group that is coupled to said second protecting group configured to prevent coupling between said amino acid and said third reporter moiety.

165. The method of claim 158, wherein said peptide is brought into contact with said mixture comprising said second reactive group or said fourth reactive group.

166. The method of claim 158, wherein said at least first signal or signal change is the same as said at least said second signal or signal change.


SEQUENCE LISTING (5 sequences)

SEQ ID NO: 1 - skipped

SEQ ID NO: 2 - protein, 14 aa, synthetic construct
  REGION 1..14: note = Synthetic peptide
KDDYAGGGAAGKDA

SEQ ID NO: 3 - skipped

SEQ ID NO: 4 - protein, 9 aa, synthetic construct
  REGION 1..9: note = Synthetic peptide
  MOD_RES 8: note = X = Propargylglycine
AKGAGRYXN

SEQ ID NO: 5 - protein, 12 aa, synthetic construct
  REGION 1..12: note = Synthetic peptide
  VARIANT 2: note = misc_feature - Xaa can be any naturally occurring amino acid<
  MOD_RES 11: note = X = Propargylglycine
AXAGANGSNGXN